(19) United States
(12) Patent Application Publication
(10) Pub. No.: US 2023/0242577 A1
(43) Pub. Date: Aug. 3, 2023

(54) METHOD FOR EXTENDING HALF-LIFE OF A PROTEIN

(71) Applicant: UBIPROTEIN, CORP., Seongnam (KR)

(72) Inventors: Kyunggon Kim, Seoul (KR); Kwang-Hyun Baek, Seoul (KR); Sung-Ryul Bae, Seongnam, Gyeonggi-do (KR); Myung-Sun Kim, Wonju, Gangwon-do (KR); Hyeonmi Kim, Suwon, Gyeonggi-do (KR); Yeeun Yoo, Guri, Gyeonggi-do (KR); Lan Li, Tangshan, Hebei (CN); Jung-Hyun Park, Daejeon (KR); Jin-Ok Kim, Jeungpyeong-gun, Chungcheongbuk-do (KR)

(21) Appl. No.: 17/990,515

(22) Filed: Nov. 18, 2022

Related U.S. Application Data
(62) Division of application No. 15/776,680, filed on May 16, 2018.

(30) Foreign Application Priority Data
Nov. 16, 2015 (KR) 10-2015-0160728

(51) Int. Cl. C07K 1/107; C07K 14/49; C07K 14/50; C07K 14/505; C07K 14/51; C07K 14/535; C07K 14/56; C07K 14/565; C07K 14/575; C07K 14/605; C07K 14/61; C07K 14/62; C07K 16/00

(52) U.S. Cl. CPC C07K 1/1075; C07K 14/49; C07K 14/50; C07K 14/505; C07K 14/51; C07K 14/535; C07K 14/56; C07K 14/565; C07K 14/575; C07K 14/5759; C07K 14/605; C07K 14/61; C07K 14/62; C07K 16/00; A61K 38/00

(57) ABSTRACT

The present invention relates to a method for prolonging half-life of a protein or a (poly)peptide by replacing one or more amino acid residues of the protein. Further, the present invention is about the protein having a prolonged half-life prepared by the method above.

### CROSS-REFERENCE TO RELATED APPLICATIONS

This application is a Division of Ser. No. 15/776,680, filed May 16, 2018, which is a U.S. national phase application, pursuant to 35 U.S.C. § 371, of PCT/KR2016/012334, filed Oct. 30, 2016, designating the United States, which claims priority to Korean Application No. 10-2015-0160728, filed Nov. 16, 2015. The entire contents of the aforementioned patent applications are incorporated herein by this reference.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 25, 2023, is named “LEE-P30009D7.xml” and is 214,680 bytes in size. The sequence listing contained in this .XML file is part of the specification and is hereby incorporated by reference herein in its entirety.

### TECHNICAL FIELD

The present invention relates to a method for prolonging half-life of a protein or a (poly)peptide by replacing one or more lysine residues of the protein related to ubiquitination, and the protein having a prolonged half-life.

### BACKGROUND ART

A protein or (poly)peptide in eukaryotic cells is degraded through two distinct pathways of lysosomal system and ubiquitin-proteasome system. The lysosomal system, in which 10 to 20% cellular proteins are decomposed, has neither substrate specificity nor precise timing controllability. That is, the lysosomal system is a process to break down especially most of extracellular proteins or membrane proteins, as surface proteins are engulfed by endocytosis and degraded by the lysosome. For the selective degradation of a protein in eukaryotic cells, ubiquitin-proteasome pathway (UPP) should be involved, wherein the target protein is first bound to ubiquitin-binding enzyme to form poly-ubiquitin chain, and then recognized and decomposed by proteasome. About 80 to 90% of eukaryotic cell proteins are degraded through UPP, and thus it is considered that the UPP regulates degradation for most of cellular proteins in eukaryotes, and presides over protein turnover and homeostasis in vivo. The ubiquitin is a small protein consisting of highly conserved 76 amino acids and it exists in all eukaryotic cells. Among the amino acid residues of the ubiquitin, the residues at positions corresponding to 6, 11, 27, 29, 33, 48 and 63 are lysines (Lysine, Lys, K), and the residues at positions 48 and 63 are known to have essential roles in the formation of poly-ubiquitin chain. The three enzymes, known generically as E1, E2 and E3, act in series to promote ubiquitination, and the ubiquitin-tagged proteins are decomposed by the 26S proteasome of ATP-dependent protein degradation complex.

As disclosed above, the ubiquitin proteasome pathway (UPP) consists of two discrete and continuous processes. One is protein tagging process in which a number of ubiquitin molecules are conjugated to the substrate proteins, and the other is degradation process where the tagged proteins are broken down by the 26S proteasome complex. The conjugation between the ubiquitin and the substrate protein is implemented by the formation of isopeptide bond between C-terminus glycine of the ubiquitin and lysine residue of the substrate, and followed by thiol-ester bond development between the ubiquitin and the substrate protein by a series of enzymes of ubiquitin-activating enzyme E1, ubiquitin-binding enzyme E2 and ubiquitin ligase E3. The E1 (ubiquitin-activating enzyme) is known to activate ubiquitin through ATP-dependent reaction mechanism. The activated ubiquitin is transferred to cysteine residue in the ubiquitin-conjugation domain of the E2 (ubiquitin-conjugating enzyme), and then the E2 delivers the activated ubiquitin to E3 ligase or to the substrate protein directly. The E3 also catalyzes stable isopeptide bond formation between lysine residue of the substrate protein and glycine of the ubiquitin. Another ubiquitin can be conjugated to the C-terminus lysine residue of the ubiquitin bound to the substrate protein, and the repetitive conjugation of additional ubiquitin moieties as such produces a poly-ubiquitin chain in which a number of ubiquitin molecules are linked to one another. If the poly-ubiquitin chain is produced, then the substrate protein is selectively recognized and degraded by the 26S proteasome.

Meanwhile, there are various kinds of proteins which have therapeutic effects in vivo. The proteins or (poly)peptides or bioactive polypeptides having therapeutic effects in vivo include, but not limited, for example, growth hormone releasing hormone (GHRH), growth hormone releasing peptide, interferons (interferon-α or interferon-β), interferon receptors, colony stimulating factors (CSFs), glucagon-like peptides, interleukins, interleukin receptors, enzymes, interleukin binding proteins, cytokine binding proteins, G-protein-coupled receptor, human growth hormone (hGH), macrophage activating factor, macrophage peptide, B cell factor, T cell factor, protein A, allergy inhibitor, cell necrosis glycoproteins, G-protein-coupled receptor, immunotoxin, lymphotoxin, tumor necrosis factor, tumor suppressors, metastasis growth factor, alpha-1 antitrypsin, albumin, alpha-lactalbumin, apolipoprotein-E, erythropoietin, highly glycosylated erythropoietin, angiopoietins, hemoglobin, thrombin, thrombin receptor activating peptide, thrombomodulin, factor VII, factor VIIa, factor VIII, factor IX, factor XIII, plasminogen activating factor, urokinase, streptokinase, hirudin, protein C, C-reactive protein, renin inhibitor, collagenase inhibitor, superoxide dismutase, leptin, platelet-derived growth factor, epithelial growth factor, epidermal growth factor, angiostatin, angiotensin, bone growth factor, bone stimulating protein, calcitonin, insulin, atriopeptin, cartilage inducing factor, fibrin-binding peptide, elcatonin, connective tissue activating factor, tissue factor pathway inhibitor, follicle stimulating hormone, luteinizing hormone, luteinizing hormone releasing hormone, nerve growth factors, parathyroid hormone, relaxin, secretin, somatomedin, insulin-like growth factor, adrenocortical hormone, glucagon, cholecystokinin, pancreatic polypeptide, gastrin releasing peptide, corticotropin releasing factor, thyroid stimulating hormone, autotaxin, lactoferrin, myostatin, receptors, receptor antagonists, cell surface antigens, virus derived vaccine antigens, monoclonal antibodies, polyclonal antibodies, and antibody fragments.

The β-trophin is known to promote the proliferation of pancreatic β cells which secrete insulin. Therefore, the β-trophin can be administered into the patients suffering from type II diabetes once or twice a year to maintain pancreatic β cells activity for controlling blood glucose level. The administration of β-trophin has a little adverse effect in comparison to the insulin administration, since the patients given β-trophin treatment can produce the insulin for themselves. Further, it was reported that the temporarily expressed β-trophin in a mouse liver promotes pancreatic β cells proliferation (Cell 153, 747758, 2013).

The growth hormone (GH), a peptide hormone, is synthesized and secreted in the anterior lobe of pituitary gland, and it relates not only to the growth of bone and cartilage, but also to the metabolism for the stimulation of adipose decomposition and protein synthesis. Thus, the growth hormone can be used for the treatment of dwarfism, wherein the dwarfism can be caused by various medical conditions including, for example, congenital heart disease, chronic lung disease, chronic kidney disease, or chronic wasting disease; inappropriate secretion of hormone due to growth hormone deficiency, hypothyroidism or diabetes; and congenital hereditary disease such as Turner syndrome. Further, it is known that the growth hormone regulates the transcription of STAT (signal transducers and activators of transcription) protein (Oncogene, 19, 2585-2597, 2000).

The insulin is known to regulate blood glucose level in a human body. Therefore, the insulin can be administered to treat type I diabetes patients who suffer from the increase of blood glucose level resulted from the functional impairment of islet cells of pancreas. In addition, the insulin can be administered into the type II diabetes patients who cannot control the blood glucose level due to the insulin receptor resistance of somatic cells, though the insulin is still normally secreted. According to the prior studies, it was reported that the insulin stimulates STAT phosphorylation in a liver, and thereby controls glucose homeostasis in the liver (Cell Metabolism 3, 267275, 2006).

The interferons, which are a group of naturally produced proteins, are produced and secreted by the immune system cells including, such as leukocyte, natural killer cell, fibrocyte and epithelial cell. The interferons are classified as 3 types, such as Type I, Type II and Type III, and the said types are determined by the receptors which are delivered by the respective proteins. Though the functional mechanism of the interferons is complicate and not yet fully understood, it is known that they regulate the immune system response to the virus, cancer and other foreign (or infectious) materials. Meanwhile, it is known that the interferons do not directly kill the virus or cancer cells, but they promote immune system response and control the function of the genes which regulate proteins secretion in the numerous cells, and thereby they suppress the growth of cancer cells. Regarding type I interferons, it is known that the IFN-α can be used for the treatment of Hepatitis B and Hepatitis C, and the IFN-β can be used to treat multiple sclerosis. Further, it was reported that the IFN-α enhances STAT-1, STAT-2 and STAT-3 (J Immunol., 187, 2578-2585, 2011), and it activates the STAT3 protein, which contributes to melanoma tumorigenesis, in melanoma cells (Euro J Cancer, 45, 1315-1323, 2009). Furthermore, it was reported that the activation of signal pathways including AKT is induced by the IFN-β treated cells (Pharmaceuticals (Basel), 3, 994-1015, 2010).

The granulocyte-colony stimulating factor (G-CSF), a glycoprotein, produces stem cell and granulocyte, and stimulates a bone marrow to secrete the stem cells and granulocytes into the blood vessel. The G-CSF is a kind of colony stimulating factors, and functions as a cytokine and a hormone as well. Further, the G-CSF acts as a neurotrophic factor, by increasing neuroplasticity and suppressing apoptosis, in addition to influencing on hematogenesis. The G-CSF receptor is expressed in the neurons of brain and spinal cord. In the central nervous system, the G-CSF induces neuron generation and increases neuroplasticity, and thereby is associated with apoptosis. Therefore, the G-CSF has been studied for use in treating neuronal diseases, such as cerebral infarction. The G-CSF stimulates the generation of granulocyte which is a kind of leukocytes. Further, the recombinant G-CSF is used for accelerating the recovery from neuropenia which is caused by chemical treatment in oncology and hematology. It was reported that the G-CSF activates STAT3 in glioma cells, and thereby involves in glioma growth (Cancer Biol Ther., 13(6), 389-400, 2012). Further, it was reported that the G-CSF is expressed in ovarian epithelial cancer cells and pathologically relates to women uterine carcinoma by regulating JAK2/STAT3 pathway (Br J Cancer, 110, 133-145, 2014).

The erythropoietin (EPO), a glycoprotein hormone, interacts with various growth factors, such as interleukin-3, interleukin-6, glucocorticoid and stem cell factors, etc. As a cytokine, erythropoietin exists in bone marrow as an erythrocyte precursor and relates to the production of erythrocyte. Furthermore, the erythropoietin relates to vasoconstriction dependent hypertension in that it up-regulates absorption of iron ion by suppressing the absorption of hepcidin hormone of iron-regulatory hormone. Further, the erythropoietin has an important roles on the neuron protection in the brain response to a neuron damage, such as myocardial infarction or stroke. In addition, the erythropoietin is known to have therapeutic effects on memory improvement, scar restore and depression. Further, it was reported that the erythropoietin level increases in lung cancer and blood cancer patients. Further, it was reported that the EPO regulates cell cycle progression through Erk1/2 phosphorylation, and thus it has effects on hypoxia (J Hematol Oncol., 6, 65, 2013).

The fibroblast growth factor-1 (FGF-1) is one of the fibroblast growth factors, and relates to embryo development, cell growth, tissue regeneration, and cancer development and transition. Further, it was reported that the FGF-1 induces cardiovascular angiogenesis in a clinical study (BioDrugs., 11(5), 301308, 1999). Since the FGF-1 promotes cell growth, it helps to maintain epidermis healthy, and thereby it strengthens skin elasticity to moisturize the skin. Further, the FGF-1 activates skin cells and brightens skin appearance, and provides milky skin. In addition, the FGF-1 is known to help rapid recovery of skin from damage or scar, and enhance protection function by fortifying skin barriers. Further, the recombinant fibroblast growth factor-1 (FGF-1) is known to enhance Erk 1/2 phosphorylation in the HEK293 cell (Nature, 513(7518), 436-439, 2014). The vascular endothelial growth factor A (VEGFA) is a signal transduction protein produced in a cell which stimulates vasculogenesis and angiogenesis, and it stores oxygen in tissues in hypoxic environment (Mol Cell Endocrinol., 397, 5157, 2014). In case of asthma and diabetes, increased serum level of the VEGF was detected (Diabetes, 48(11), 22292239, 2013). The VEGF functions in embryo development, a new vessel generation after damage, and a new vessel generation penetrating muscle and the blocked vessel after exercise. Meanwhile, the over-expression of VEGF results in diseases or disorders. For example, the solid cancer does not grow further if the blood inflow is blocked, but the cancer grows continuously and metastasis is developed if the VEGF is expressed. Further, the VEGF is known as an important factor for the growth and proliferation of endothelial cells and involves in angiogenesis development in cancer cells. In particular, it was reported that the PI3K/Akt/HIF-la signal transduction pathway relates angiogenesis development by the VEGF in cancer cells (Carcinogenesis, 34, 426-435, 2013). Further, the VEGF is known to induce AKT phosphorylation (Kidney Int., 68, 1648-1659, 2005).

The appetite suppressing protein (Leptin) and the appetite stimulating hormone (Ghrelin) are secreted in adipose tissues. The Leptin is a circulating hormone (16 kDa) (Cell Res., 10, 81-92, 2000) and has important roles on immunity, reproduction and hematogenesis. The Ghrelin, which is secreted from adipose tissues through the growth hormone secretagogue receptor (GHS-R) and stimulates appetite, is a stomach-peptide consisting of 28 amino acids (J Endocrinol., 192, 313323, 2007; Nature, 442, 656-660, 1999), and is formed from preproghrelin (Pediatr Res., 65, 3944, 2009; J Biol Chem., 281(50), 3886738870, 2006).

The Leptin is a hormone providing fullness signal not to have foods any more, and the impaired Leptin hormone secretion is known to stimulate appetite. It was reported that the fructose interferes insulin secretion and reduces the Leptin secretion, while it promotes the secretion of Ghrelin to increase appetite (J Biol Chem., 277(7), 5667-5674, 2002; I.J.S.N., 7(1), 06-15, 2016). Further, the appetite suppressing protein was reported to increase AKT phosphorylation in breast cancer cells (Cancer Biol Ther., 16(8), 1220-1230, 2015), and stimulates cancer cells growth in PI3K/AKT signal transduction pathways in uterine cancer (Int J Oncol., 49(2), 847, 2016). Further, the Leptin was known to stimulate cancer cells growth in uterine cancers through PI3K/AKT signal transduction (Int J Oncol., 49(2), 847, 2016).

The appetite stimulating hormone (Ghrelin) was known to regulate cell growth through the growth hormone secretagogue receptor (GHS-R), and enhance STAT3 by way of calcium regulation in vivo (Mol Cell Endocrinol., 285, 19-25, 2008).

The glucagon-like peptide-1 (GLP-1), an incretin hormone, which is secreted from L cells of the ileum and the large intestine, increases insulin secretion dependent on the glucose concentration, and thus it prevents hypoglycemia. Therefore, the GLP-1 can be used for the treatment of type II diabetes (Pharmaceuticals (Basel), 3(8), 2554-2567, 2010; Diabetologia, 36(8), 741-744, 1993). Further, the GLP-1 induces hypokinesis of the upper digestive organs and suppresses appetite, and can stimulate the proliferation of the existing pancreas β cells (Endocr Rev., 16(3), 390-410, 1995; Endocrinology, 141(12), 4600-4605, 2000; Dig Dis Sci., 38(4), 665-673, 1993; Am J Physiol., 273(5 Pt 1), E981-988, 1997). However, 2 minutes of short in vivo half-life of the GLP-1 is a disadvantage for the development of medicinal agent by using the GLP1. The glucagon-like peptide-1 (GLP-1) regulates homeostasis and plays critical roles on insulin resistance, and thereby it has been used as diabetes therapeutic agent. Further, it was reported that the GLP-1 induces STAT3 activation (Biochem Biophys Res Commun., 425(2), 304-308, 2012).

The BMP-2, one of the TGF-β superfamily, contributes to the formation of cartilage and bone, and has critical roles in cell growth, cell death and cell differentiation (Genes Dev., 10, 1580-1594, 1996; Development, 122, 3725-3734, 1996; J Biol Chem., 274, 26503-26510, 1999; J Exp Med., 189, 1139-1147, 1999). Further, it was reported that the BMP-2 can be used as a treating agent for multiple sclerosis (Blood, 96(6), 2005-2011, 2000; Leuk Lymphoma., 43(3), 635-639, 2002).

Immunoglobulin G (IgG) is a type of antibody and it is the main type of antibody found in blood and extracellular fluid allowing it to control infection of body tissues, and is secreted as a monomer that is small in size allowing it to easily perfuse tissues (Basic Histology, McGraw-Hill, ISBN 0-8385-0590-2, 2003). IgG is used to treat immune deficiencies, autoimmune disorders, and infections (Proc Natl Acad Sci USA., 107(46), 19985-19990, 2010).

The protein therapeutic agents relating to homeostasis in vivo have various adverse effects, such as increasing the risk for cancer inducement. For example, possible inducement of thyroid cancer was raised for the incretin degrading enzyme (DPP-4) (Dipeptidyl peptidase-4) inhibitors family therapeutic agents, and insulin glargine was known to increase the breast cancer risk. Further, it was reported that continuous or excessive administration of the growth hormone into the patients suffering from a disease of growth hormone secretion disorder is involved in diabetes, microvascular disorders and premature death of the patients. In this regard, there have been broad studies to reduce such adverse and side effects of the therapeutic proteins. To prolong half-life of the proteins was suggested as a method to minimize the risk of the adverse and side effects of the therapeutic proteins. For this purpose, various methods have been disclosed. In this regard, we, inventors have studied to develop a novel method for prolonging half-life of the proteins in vivo and/or in vitro and completed the present invention by replacing one or more lysine residues related to ubiquitination of the therapeutic proteins or (poly)peptide to prevent the proteins or (poly)peptide degradation through ubiquitine-proteasome system.

The teachings of all patents, published applications and references cited herein are incorporated by reference in their entirety.

### SUMMARY

The purpose of the present invention is to enhance half-life of the proteins or (poly)peptide.

Further, another purpose of the present invention is to provide a therapeutic protein having prolonged half-life.

Further, another purpose of the present invention is to provide a pharmaceutical composition comprising the protein having prolonged half-life as a pharmacological active ingredient.

In order to achieve the purpose, this invention provides a method for extending protein half-life in vivo and/or in vitro by replacing one or more lysine residues on the amino acids of the protein.

In the present invention, the lysine residue can be replaced by conservative amino acid. The term “conservative amino acid replacement” means that an amino acid is replaced by another amino acid which is different from the amino acid to be replaced but has similar chemical features, such as charge or hydrophobic property. The functional features of a protein are not essentially changed by the amino acid replacement using the corresponding conservative amino acid, in general. For example, amino acids can be classified according to the side chains having similar chemical properties, as follows: \textcircled{1} aliphatic side chain: Glycine, Alanine, Valine, Leucine, and Isoleucine; \textcircled{2} aliphatic-hydroxyl side chain: Serine and Threonine; \textcircled{3} Amide containing side chain: Asparagine and Glutamine; \textcircled{4} aromatic side chain: Phenyl alanine, Tyrosine, Tryptophan; \textcircled{5} basic side chain: Lysine, Arginine and Histidine; \textcircled{6} Acidic side chain; Aspartate and Glutamate; and \textcircled{7} sulfur-containing side chain: Cysteine and Methionine.

In the present invention, the lysine residue can be substituted with arginine or histidine which contains basic side chain. Preferably, the lysine residue is replaced by arginine.

In accordance with the present invention, the mutated protein of which one or more lysine residues are substituted with arginine has significantly prolonged half-life, and thus can remain for a long time.

### BRIEF DESCRIPTION OF DRAWINGS

FIG. 1 shows the structure of β-trophin expression vector. FIG. 1 discloses SEQ ID NO: 111.

FIG. 2 represents the results of cloning PCR products for the β-trophin gene.

FIG. 3 shows the expression β-trophin plasmid genes in the HEK-293T cells.

FIG. 4 explains the proteolytic pathway of the β-trophin via ubiquitination assay.

FIG. 5 shows the ubiquitination levels of the substituted β-trophin of which lysine residues are replace by arginines, in comparison to the wild type.

FIG. 6 shows the β-trophin's half-life change after the treatment with protein synthesis inhibitor cyclohexamide (CHX).

FIG. 7 shows the results for the JAK-STAT signal transduction like effects.

FIG. 8 shows the structure of growth hormone expression vector. FIG. 8 discloses SEQ ID NO: 112.

FIG. 9 represents the results of cloning PCR products for the growth hormone gene.

FIG. 10 shows the expression growth hormone plasmid genes in the HEK-293T cells.

FIG. 11 explains the proteolytic pathway of the growth hormone via ubiquitination assay.

FIG. 12 shows the ubiquitination levels of the substituted growth hormone of which lysine residue(s) is replace by arginine(s), in comparison to the wild type.

FIG. 13 shows the growth hormone half-life change after the treatment with protein synthesis inhibitor cyclohexamide (CHX).

FIG. 14 shows the results for the JAK-STAT signal transduction like effects.

FIG. 15 shows the structure of insulin expression vector. FIG. 15 discloses SEQ ID NO: 113.

FIG. 16 represents the results of cloning PCR products for the insulin gene.

FIG. 17 shows the expression of insulin plasmid genes in the HEK-293T cells.

FIG. 18 explains the proteolytic pathway of the insulin via ubiquitination assay.

FIG. 19 shows the ubiquitination levels of the substituted insulin mutants of which lysine residue(s) is replace by arginine(s), in comparison to the wild type.

FIG. 20 shows the insulin half-life change after the treatment with protein synthesis inhibitor cyclohexamide (CHX).

FIG. 21 shows the results for the JAK-STAT signal transduction like effects.

FIG. 22 shows the structure of interferon-α expression vector. FIG. 22 discloses SEQ ID NO: 114.

FIG. 23 represents the results of cloning PCR products for the interferon-α gene.

FIG. 24 shows the expression of interferon-α plasmid genes in the HEK-293T cells.

FIG. 25 explains the proteolytic pathway of the interferon-α via ubiquitination assay.

FIG. 26 shows the ubiquitination levels of the substituted interferon-α of which lysine residue(s) is replace by arginine(s), in comparison to the wild type.

FIG. 27 shows the interferon-α half-life change after the treatment with protein synthesis inhibitor cyclohexamide (CHX).

FIG. 28 shows the results for the JAK-STAT signal transduction like effects.

FIG. 29 shows the structure of G-CSF expression vector. FIG. 29 discloses SEQ ID NO: 115.

FIG. 30 represents the results of cloning PCR products for the G-CSF gene.

FIG. 31 shows the expression of G-CSF plasmid genes in the HEK-293T cells.

FIG. 32 explains the proteolytic pathway of the G-CSF via ubiquitination assay.

FIG. 33 shows the ubiquitination levels of the substituted G-CSF of which lysine residues are replace by arginines, in comparison to the wild type.

FIG. 34 shows the G-CSF half-life change after the treatment with protein synthesis inhibitor cyclohexamide (CHX).

FIG. 35 shows the results for the JAK-STAT signal transduction like effects.

FIG. 36 shows the structure of interferon-β expression vector. FIG. 36 discloses SEQ ID NO: 116.

FIG. 37 represents the results of cloning PCR products for the interferon-β gene.

FIG. 38 shows the expression of interferon-β plasmid genes in the HEK-293T cells.

FIG. 39 explains the proteolytic pathway of the interferon-β via ubiquitination assay.

FIG. 40 shows the ubiquitination levels of the substituted interferon-β of which lysine residues are replace by arginines, in comparison to the wild type.

FIG. 41 shows the interferon-β half-life change after the treatment with protein synthesis inhibitor cyclohexamide (CHX).

FIG. 42 shows the results for the JAK-STAT and PI3K/AKT signal transduction like effects.

FIG. 43 shows the structure of erythropoietin expression vector. FIG. 43 discloses SEQ ID NO: 117.

FIG. 44 represents the results of cloning PCR products for the erythropoietin gene.

FIG. 45 shows the expression of erythropoietin plasmid genes in the HEK-293T cells.

FIG. 46 explains the proteolytic pathway of the erythropoietin via ubiquitination assay.

FIG. 47 shows the ubiquitination levels of the substituted erythropoietin of which lysine residues are replace by arginines, in comparison to the wild type.

FIG. 48 shows the erythropoietin half-life change after the treatment with protein synthesis inhibitor cyclohexamide (CHX).

FIG. 49 shows the results for the MAPK/ERK signal transduction like effects.

FIG. 50 shows the structure of BMP2 expression vector. FIG. 50 discloses SEQ ID NO: 118.

FIG. 51 represents the results of cloning PCR products for the BMP2 gene.

FIG. 52 shows the expression of BMP2 plasmid genes in the HEK-293T cells.

FIG. 53 explains the proteolytic pathway of the BMP2 via ubiquitination assay.

FIG. 54 shows the ubiquitination levels of the substituted BMP2 of which lysine residue(s) are replace by arginine(s), in comparison to the wild type.

FIG. 55 shows the BMP2 half-life change after the treatment with protein synthesis inhibitor cyclohexamide (CHX).

FIG. 56 shows the results for the JAK-STAT signal transduction like effects.

FIG. 57 shows the structure of fibroblast growth factor-1 (FGF-1) expression vector. FIG. 57 discloses SEQ ID NO: 119.

FIG. 58 represents the results of cloning PCR products for the FGF-1 gene.

FIG. 59 shows the expression of FGF-1 plasmid genes in the HEK-293T cells.

FIG. 60 explains the proteolytic pathway of the FGF-1 via ubiquitination assay.

FIG. 61 shows the ubiquitination levels of the substituted FGF-1 of which lysine residue(s) are replace by arginine(s), in comparison to the wild type.

FIG. 62 shows the FGF-1 half-life change after the treatment with protein synthesis inhibitor cyclohexamide (CHX).

FIG. 63 shows the results for the MAPK/ERK signal transduction like effects.

FIG. 64 shows the structure of Leptin expression vector. FIG. 64 discloses SEQ ID NO: 120.

FIG. 65 represents the results of cloning PCR products for the Leptin gene.

FIG. 66 shows the expression of Leptin plasmid genes in the HEK-293T cells.

FIG. 67 explains the proteolytic pathway of the Leptin via ubiquitination assay.

FIG. 68 shows the ubiquitination levels of the substituted Leptin of which lysine residue(s) is replace by arginine(s), in comparison to the wild type.

FIG. 69 shows the Leptin half-life change after the treatment with protein synthesis inhibitor cyclohexamide (CHX).

FIG. 70 shows the results for the PI3K/AKT signal transduction like effects.

FIG. 71 shows the structure of Vascular endothelial growth factor A (VEGFA) expression vector. FIG. 71 discloses SEQ ID NO: 121.

FIG. 72 represents the results of cloning PCR products for the VEGFA gene.

FIG. 73 shows the expression of VEGFA plasmid genes in the HEK-293T cells.

FIG. 74 explains the proteolytic pathway of the VEGFA via ubiquitination assay.

FIG. 75 shows the ubiquitination levels of the substituted VEGFA of which lysine residue(s) is replace by arginine(s), in comparison to the wild type.

FIG. 76 shows the VEGFA half-life change after the treatment with protein synthesis inhibitor cyclohexamide (CHX).

FIG. 77 shows the results for the JAK-STAT and PI3K/AKT signal transduction like effects.

FIG. 78 shows the structure of Ghrelin/obestatin prepropeptide (Prepro-GHRL) expression vector. FIG. 78 discloses SEQ ID NO: 122.

FIG. 79 represents the results of cloning PCR products for the Prepro-GHRL gene.

FIG. 80 shows the expression of Prepro-GHRL plasmid genes in the HEK-293T cells.

FIG. 81 explains the proteolytic pathway of the Prepro-GHRL via ubiquitination assay.

FIG. 82 shows the ubiquitination levels of the substituted Prepro-GHRL of which lysine residue(s) are replace by arginine(s), in comparison to the wild type.

FIG. 83 shows the Prepro-GHRL half-life change after the treatment with protein synthesis inhibitor cyclohexamide (CHX).

FIG. 84 shows the results for the JAK-STAT signal transduction like effects.

FIG. 85 shows the structure of GHRL expression vector. FIG. 85 discloses SEQ ID NO: 123.

FIG. 86 represents the results of cloning PCR products for the GHRL gene.

FIG. 87 shows the expression of GHRL plasmid genes in the HEK-293T cells.

FIG. 88 explains the proteolytic pathway of the GHRL via ubiquitination assay.

FIG. 89 shows the ubiquitination levels of the substituted GHRL of which lysine residue(s) is replace by arginine(s), in comparison to the wild type.

FIG. 90 shows the GHRL half-life change after the treatment with protein synthesis inhibitor cyclohexamide (CHX).

FIG. 91 shows the results for the JAK-STAT signal transduction like effects.

FIG. 92 shows the structure of Glucagon-like peptide-1 (GLP-1) expression vector. FIG. 92 discloses SEQ ID NO: 124.

FIG. 93 represents the results of cloning PCR products for the GLP-1 gene.

FIG. 94 shows the expression of GLP-1 plasmid genes in the HEK-293T cells.

FIG. 95 explains the proteolytic pathway of the GLP-1 via ubiquitination assay.

FIG. 96 shows the ubiquitination levels of the substituted GLP-1 of which lysine residue(s) is replace by arginine(s), in comparison to the wild type.

FIG. 97 shows the GLP-1 half-life change after the treatment with protein synthesis inhibitor cyclohexamide (CHX).

FIG. 98 shows the results for the JAK-STAT signal transduction like effects.

FIG. 99 shows the structure of IgG heavy chain expression vector. FIG. 99 discloses SEQ ID NO: 125.

FIG. 100 represents the results of cloning for the IgG heavy chain gene.

FIG. 101 shows the expression of IgG heavy chain plasmid genes in the HEK-293T cells.

FIG. 102 explains the proteolytic pathway of the IgG heavy chain via ubiquitination assay.

FIG. 103 shows the ubiquitination levels of the substituted IgG heavy chain of which lysine residue(s) is replace by arginine(s), in comparison to the wild type.

FIG. 104 shows the IgG heavy chain half-life change after the treatment with protein synthesis inhibitor cyclohexamide (CHX).

FIG. 105 shows the structure of IgG light chain expression vector. FIG. 105 discloses SEQ ID NO: 126.

FIG. 106 represents the results of cloning for the IgG light chain gene.

FIG. 107 shows the expression of IgG light chain plasmid genes in the HEK-293T cells.

FIG. 108 explains the proteolytic pathway of the IgG light chain via ubiquitination assay.

FIG. 109 shows the ubiquitination levels of the substituted IgG light chain of which lysine residue(s) is replace by arginine(s), in comparison to the wild type.

FIG. 110 shows the IgG light chain half-life change after the treatment with protein synthesis inhibitor cyclohexamide (CHX).

Hereinafter, the present invention will be described in more detail with reference to Examples. It should be understood that these examples are not to be in any way construed as limiting the present invention.

### DETAILED DESCRIPTION

In one embodiment of the present invention, the protein is β-trophin. In the β-trophin amino acid sequence (SEQ ID NO: 1), at least one lysine residues at positions corresponding to 62, 124, 153 and 158 from the N-terminus are substituted with arginine. As a result, a β-trophin having increased in vivo and/or in vitro half-life is provided. Further, a pharmaceutical composition comprising the substituted β-trophin for preventing and/or treating diabetes and obesity is provided (Cell, 153(4), 747758, 2013; Cell Metab., 18(1), 5-6, 2013; Front Endocrinol (Lausanne), 4, 146, 2013).

In another embodiment of the present invention, the protein is growth hormone. In this growth hormone's amino acid sequence (SEQ ID NO: 10), at least one lysine residues at positions corresponding to 64, 67, 96, 141, 166, 171, 184, 194 and 198 from the N-terminus are substituted with arginine. As a result, a growth hormone with enhanced in vivo and/or in vitro half-life is provided. Further, a pharmaceutical composition comprising the substituted growth hormone for preventing and/or treating dwarfism, Kabuki syndrome and Kearns-Sayre syndrome (KSS) is provided (J Endocrinol Invest., 39(6), 667-677, 2016; J Pediatr Endocrinol Metab., 2016, [Epub ahead of print]; Horm Res Paediatr. 2016, [Epub ahead of print]).

In another embodiment of the present invention, the protein is insulin. In this insulin's amino acid sequence (SEQ ID NO: 17), at least one lysine residues at positions corresponding to 53 and 88 from the N-terminus are replaced by arginine. As a result, an insulin having enhanced half-life is provided. Further, a pharmaceutical composition comprising the substituted insulin for preventing and/or treating diabetes is provided.

In yet another embodiment of the present invention, the protein is an interferon-α. In this interferon-α's amino acid sequence (SEQ ID NO: 22), at least one lysine residues at positions corresponding to 17, 54, 72, 93, 106, 135, 144, 154, 156, 157 and 187 from the N-terminus are replaced by arginine. As a result, an interferon-α having enhanced in vivo and/or in vitro half-life is provided. Further, a pharmaceutical composition comprising the substituted interferon-α is provided for preventing and/or treating immune disease comprising multiple sclerosis, autoimmune disease, rheumatoid arthritis; and/or cancer comprising solid cancer and/or blood cancer; and/or infectious disease comprising virus infection, HIV related disease and Hepatitis C. disease or disorder requiring interferon-α treatment is provided (Ann Rheum Dis., 42(6), 672-676, 1983; Memo., 9, 63-65, 2016).

In yet another embodiment of the present invention, the protein is G-CSF. In the G-CSF's amino acid sequence (SEQ ID NO: 31), at least one lysine residues at positions corresponding to 11, 46, 53, 64 and 73 from the N-terminus are replaced by arginine. As a result, a G-CSF which has prolonged in vivo and/or in vitro half-life is provided. Further, a pharmaceutical composition comprising G-CSF for preventing and/or treating neutropenia is provided (EMBO Mol Med. 2016, [Epub ahead of print]).

In yet another embodiment of the present invention, the protein is interferon-β. In the interferon-β's amino acid sequence (SEQ ID NO: 36), at least one lysine residues at positions corresponding to 4, 40, 54, 66, 73, 120, 126, 129, 136, 144, 155, and 157 from the N-terminus are replaced by arginine. As a result, interferon-β which has prolonged in vivo and/or in vitro half-life is provided. Further, a pharmaceutical composition comprising the substituted interferon-β is provided for preventing and/or treating immune disease comprising multiple sclerosis, autoimmune disease, rheumatoid arthritis; and/or cancer comprising solid cancer and/or blood cancer; and/or infectious disease comprising virus infection, HIV related disease and Hepatitis C.

In yet another embodiment of the present invention, the protein is erythropoietin. In the erythropoietin's amino acid sequence (SEQ ID NO: 43), at least one lysine residues at positions corresponding to (47, 72, 79, 124, 143, 167, 179 and 181 from the N-terminus are substituted with arginine. As a result, erythropoietin having increased in vivo and/or in vitro half-life is provided. Further, the substituted erythropoietin-containing pharmaceutical composition is provided to prevent and/or treat anemia which is caused by chronic renal failure, surgical operation, and cancer or cancer treatment, etc.

In yet another embodiment of the present invention, the protein is bone morphogenetic protein-2 (BMP2). In the BMP2's amino acid sequence (SEQ ID NO: 52), at least one lysine residues at positions corresponding to 32, 64, 127, 178, 185, 236, 241, 272, 278, 281, 285, 287, 290, 293, 297, 355, 358, 379 and 383 from the N-terminus are substituted with arginine. As a result, BMP2 having increased half-life is provided. Further, the substituted BMP2-containing pharmaceutical composition is provided to prevent and/or treat anemia and bone diseases (Cell J., 17(2), 193-200, 2015; Clin Orthop Relat Res., 318, 222-230, 1995).

In yet another embodiment of the present invention, the protein is fibroblast growth factor-1 (FGF-1). In the FGF-1's amino acid sequence (SEQ ID NO: 61), at least one lysine residues at positions corresponding to 15, 24, 25, 27, 72, 115, 116, 120, 127, 128, 133 and 143 from the N-terminus are substituted with arginine. As a result, the FGF-1 having increased half-life is provided. Further, the substituted FGF-1 containing pharmaceutical composition is provided to prevent and/or treat neuron diseases.

In yet another embodiment of the present invention, the protein is appetite suppressant hormone (Leptin). In the appetite suppressant hormone (Leptin)'s amino acid sequence (SEQ ID NO: 66), at least one lysine residues at positions corresponding to 26, 32, 36, 54, 56, 74 and 115 from the N-terminus are substituted with arginine. As a result, the appetite suppressant hormone (Leptin) having increased half-life is provided. Further, the substituted appetite suppressant hormone (Leptin) containing pharmaceutical composition for preventing and/or treating brain disease, heart disease and/or obesity is provided (Ann N Y Acad Sci., 1243, 1529, 2011; J Neurochem., 128(1), 162-172, 2014; Clin Exp Pharmacol Physiol., 38(12), 905-913, 2011).

In yet another embodiment of the present invention, the protein is VEGFA. In the VEGFA's amino acid sequence (SEQ ID NO: 75), at least one lysine residues at positions corresponding to 22, 42, 74, 110, 127, 133, 134, 141, 142, 147, 149, 152, 154, 156, 157, 169, 180, 184, 191 and 206 from the N-terminus are substituted with arginine. As a result, the VEGFA having increased half-life and the pharmaceutical composition comprising thereof is provided to prevent and/or treat anti-aging, hair growth, scar and/or angiogenesis relating disease.

In yet another embodiment of the present invention, the protein is appetite stimulating hormones precursor, Ghrelin/Obestatin Preprohormone (prepro-GHRL). In the amino acid sequence (SEQ ID NO: 80) of the appetite stimulating hormones precursor, a lysine residue at position corresponding to 39, 42, 43, 47, 85, 100, 111 and 117 from the N-terminus is substituted with arginine. As a result, an appetite stimulating hormone precursor showing increased half-life is provided. Further, a pharmaceutical composition comprising the substituted appetite stimulating hormone precursor is provided to prevent and/or treat obesity, malnutrition, and/or eating disorder, such as anorexia nervosa.

In yet another embodiment of the present invention, the protein is appetite stimulating hormone (Ghrelin). In the amino acid sequence (SEQ ID NO: 83) of the Ghrelin, at least one lysine residues at positions corresponding to 39, 42, 43 and 47 from the N-terminus are replaced by arginine. Thus, an appetite stimulating hormone (Ghrelin) having increased half-life is provided. Further, a pharmaceutical composition comprising the substituted Ghrelin is provided to prevent and/or treat obesity, malnutrition, and/or eating disorder, such as anorexia nervosa.

In yet another embodiment of the present invention, the protein is glucagon like peptide-1 (GLP-1). In the amino acid sequence (SEQ ID NO: 92) of the GLP-1, at least one lysine residues at positions corresponding to 117 and 125 from the N-terminus are replaced by arginine. As a result, a GLP-1 having increased half-life and the pharmaceutical composition comprising thereof for preventing and/or treating diabetes is provided.

In yet another embodiment of the present invention, the protein is IgG. In the amino acid sequence (SEQ ID NO: 97) of the IgG heavy chain, at least one lysine residues at positions corresponding to 49, 62, 84, 95, 143, 155, 169, 227, 232, 235, 236, 240, 244, 268, 270, 296, 310, 312, 339, 342, 344, 348, 356, 360, 362, 382, 392, 414, 431, 436 and 461 from the N-terminus are replaced by arginine. As a result, the IgG having enhanced half-life and the pharmaceutical composition comprising thereof are provided to prevent and/or treat cancer.

In yet another embodiment of the present invention, the protein is IgG. In the amino acid sequence (SEQ ID NO: 104) of the IgG light chain, at least one lysine residues at positions corresponding to 61, 64, 67, 125, 129, 148, 167, 171, 191, 205, 210, 212 and 229 from the N-terminus are replaced by arginine. As a result, the IgG having enhanced half-life and the pharmaceutical composition comprising thereof are provided to prevent and/or treat cancer.

In the present invention, site-directed mutagenesis is employed to substitute lysine residue with arginine (R) residue of the amino acid sequence of the protein. According to this method, primer sets are prepared using DNA sequences to induce site-directed mutagenesis, and then PCR is performed under the certain conditions to produce mutant plasmid DNAs.

In the present invention, the degree of ubiquitination was determined by transfecting a cell line with the target protein by using immunoprecipitation. If the ubiquitination level increases in the transfected cell line after MG132 reagent treatment, it is understood that the target protein is degraded through ubiquitin-proteasome pathway.

The pharmaceutical composition of the president is invention can be administered into a body through various ways including oral, transcutaneous, subcutaneous, intravenous, or intramuscular administration, and more preferably can be administered as an injection type preparation. Further, the pharmaceutical composition of the present invention can be formulated using the method well known to the skilled in the art to provide rapid, sustained or delayed release of the active ingredient following the administration thereof. The formulations may be in the form of a tablet, pill, powder, sachet, elixir, suspension, emulsion, solution, syrup, aerosol, soft and hard gelatin capsule, sterile injectable solution, sterile packaged powder and the like. Examples of suitable carriers, excipients, and diluents are lactose, dextrose, sucrose, mannitol, xylitol, erythritol, maltitol, starches, gum acacia, alginates, gelatin, calcium phosphate, calcium silicate, cellulose, methyl cellulose, microcrystalline cellulose, polyvinyl pyrrolidone, water, methylhydroxybenzoates, propylhydroxybenzoates, talc, magnesium stearate and mineral oil. Further, the formulations may additionally include fillers, anti-agglutinating agents, lubricating agents, wetting agents, favoring agents, emulsifiers, preservatives and the like.

Examples of suitable carriers, excipients, and diluents are lactose, dextrose, sucrose, mannitol, xylitol, erythritol, maltitol, starches, gum acacia, alginates, gelatin, calcium phosphate, calcium silicate, cellulose, methyl cellulose, microcrystalline cellulose, polyvinyl pyrrolidone, water, methylhydroxybenzoates, propylhydroxybenzoates, talc, magnesium stearate and mineral oil. Further, the formulations may additionally include fillers, anti-agglutinating agents, lubricating agents, wetting agents, favoring agents, emulsifiers, preservatives and the like.

As used herein, the singular forms “a,” “an,” and “the” are intended to include the plural forms as well, unless the context clearly indicates otherwise. Furthermore, to the extent that the terms “including,” “includes,” “having,” “has,” “with,” “such as,” or variants thereof, are used in either the specification and/or the claims, such terms are not limiting and are intended to be inclusive in a manner similar to the term “comprising”. In the present invention, the “bioactive polypeptide or protein” is the (poly)peptide or protein representing useful biological activity when it is administered into a mammal including human.

### EXAMPLES

The following examples provide illustrative embodiments. In light of the present disclosure and the general level of skill in the art, those of skill will appreciate that the following examples are intended to be exemplary only and that numerous changes, modifications, and alterations can be employed without departing from the scope of the presently claimed subject matter.

### Example 1: Analysis of β-Trophin Ubiquitination and Half-Life Prolonging, and Examination of Signal Transduction in a Cell

1. β-Trophin Expression Vector Cloning and Protein Expression

(1) β-Trophin Expression Vector Cloning

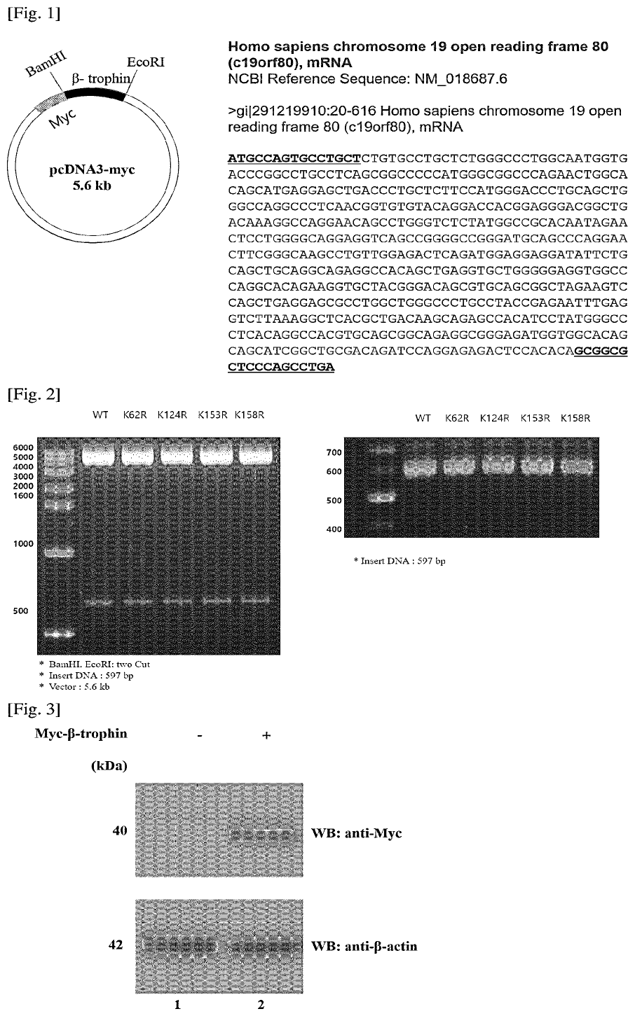

RNA was purified and extracted from HepG2 (ATCC, HB-8065) using Trizol and chloroform to clone β-trophin. Then, a single strand DNA was synthesized by using SuperScript™ First-Strand cDNA Synthesis System (Invitrogen, Grand Island, N.Y.). The β-trophin was amplified by PCR using the synthesized cDNA above as a template. The obtained β-trophin DNA amplification product was treated with BamHI and EcoRI, and then ligated to pcDNA3-myc (5.6 kb) vector previously digested with the same enzymes (FIG. 1, β-trophin amino acid sequence: SEQ ID NO: 1). Then, agarose gel electrophoresis was carried out to confirm the presence of the DNA insert, after restriction enzyme digestion of the cloned vector (FIG. 2). The PCR conditions are as follows: Step 1: at 94° C. for 3 minutes (1 cycle); Step 2: at 94° C. for 30 seconds; at 58° C. for 30 seconds; at 72° C. for 1 minute (25 cycles); and Step 3: at 72° C. for 10 minutes (1 cycle), and then held at 4° C. The nucleotide sequences in underlined bold letters in FIG. 1 indicate the primer sets used for the PCR to confirm the cloned sites (FIG. 2). For the analysis of protein expression, western blot was performed with anti-myc antibody (9E10, sc-40) to myc of pcDNA3-myc vector in the map of FIG. 1. The western blot result showed that the β-trophin protein was expressed well. The normalization with actin assured that proper amount of protein was loaded (FIG. 3).

(2) Lysine (Lysine, K) Residue Substitution

Lysine residue was replaced by arginine (Arginine, R) using site-directed mutagenesis. The following primer sets were used for PCR to produce substituted plasmid DNAs.

(β-trophin K62R)
FP
(SEQ No. 2)
5′-AGGGACGGCTGACAAGGGCCAGGAA-3′,
RP
(SEQ No. 3)
5′-CCAGGCTGTTCCTGGCCCTTGT CAGC-3′;
(β-trophin K124R)
FP
(SEQ No. 4)
5′-GGCACAGAGGGTGCTACGGGACAGC-3′,
RP
(SEQ No. 5)
5′-CGTAGCACCCTCTGTGCCTGGGCCA-3′;
(β-trophin K153R)
FP
(SEQ No. 6)
5′-GAATTTGAGGTCTTAAGGGCTCACGC-3′,
RP
(SEQ No. 7)
5′-CTTGTC AGCGTGAGCCCTTAAGACCTC-3′;
and
(β-trophin K158R)
FP
(SEQ No. 8)
5′-GCTCACGCTGACAGGCAGAGCCACAT-3′,
RP
(SEQ No. 9)
5′-CCATAGGATGTGGCTCTGCCTGTCAGC-3′.

Four plasmid DNAs each of which one or more lysine residues were substituted with arginine (K→R) were prepared by using pcDNA3-myc-β-trophin as a template (Table 1).

TABLE 1

| Lysine(K) residue | β-trophin construct, replacement |
|---|---|
| site | of K with R |
| 62 | pcDNA3-myc-β-trophin (K62R) |
| 124 | pcDNA3-myc-β-trophin (K124R) |
| 153 | pcDNA3-myc-β-trophin (K153R) |
| 158 | pcDNA3-myc-β-trophin (K158R) |

2. In Vivo Ubiquitination Analysis

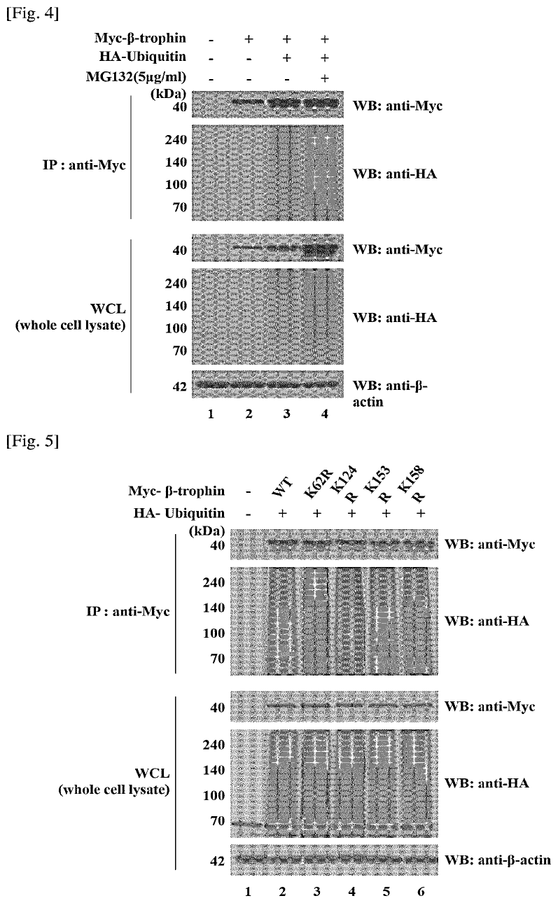

The HEK 293T cell (ATCC, CRL-3216) was transfected with the plasmid encoding pcDNA3-myc-β-trophin WT and pMT123-HA-ubiquitin (J Biol Chem., 279(4), 2368-2376, 2004; Cell Research, 22, 873885, 2012; Oncogene, 22, 12731280, 2003; Cell, 78, 787-798, 1994). For the analysis of the degree of ubiquitination, pcDNA3-myc-β-trophin WT 2 μg and pMT123-HA-ubiquitin DNA 1 μg were co-transfected into the cells. 24 hrs after the transfection, the cells were treated with MG132 (proteasome inhibitor, 5 μg/ml) for 6 hrs, thereafter immunoprecipitation analysis was carried out (FIG. 4). Then, the HEK 293T cell was transfected with the plasmids encoding pc-β-trophin WT, pcDNA3-myc-β-trophin mutant (K62R), pcDNA3-myc-β-trophin mutant (K124R), pcDNA3-myc-β-trophin mutant (K153R) and pcDNA3-myc-β-trophin mutant (K158R), respectively. For the analysis of the ubiquitination level, the cells were co-transfected with 1 μg of pMT123-HA-ubiquitin DNA, and with respective 2 μg of pcDNA3-myc-β-trophin WT, pcDNA3-myc-β-trophin mutant (K62R), pcDNA3-myc-β-trophin mutant (K124R), pcDNA3-myc-β-trophin mutant (K153R) and pcDNA3-myc-β-trophin mutant (K158R). Next, 24 hrs after the transfection, the immunoprecipitation was carried out (FIG. 5). The protein sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Then, the mixture was incubated at 4° C., overnight. The immunoprecipitant was separated, following the reaction with A/G bead (Santa Cruz Biotechnology) at 4° C., for 2 hrs. Next, the separated immunoprecipitant was washed twice with buffering solution.

The protein sample was separated by SDS-PAGE, after mixing with 2×SDS buffer and heating at 100° C., for 7 minutes. The separated proteins were moved to polyvinylidene difluoride (PVDF) membrane, and then developed with ECL system (Western blot detection kit, ABfrontier, Seoul, Korea) using anti-mouse secondary antibody (Peroxidase-labeled antibody to mouse IgG (H+L), KPL, 074-1806) and blocking solution which comprises anti-myc (9E10, sc-40), anti-HA (Santa Cruz Biotechnology, sc-7392) and anti-3-actin (Santa Cruz Biotechnology, sc-47778) in 1:1,000 (w/w). As a result, when immunoprecipitation was performed by using anti-myc (9E10, sc-40), poly-ubiquitin chain was formed by the binding of the ubiquitin to pcDNA3-myc-β-trophin WT, and thereby intense band indicating the presence of smear ubiquitin was produced (FIG. 4, lanes 3 and 4). Further, when the cells were treated with MG132 (proteasome inhibitor, 5 μg/ml) for 6 hrs, poly-ubiquitin chain formation was increased, and thus the more intense band indicating ubiquitin was shown (FIG. 4, lane 4). As for the pcDNA3-myc-β-trophin mutant (K62R), pcDNA3-myc-β-trophin mutant (K153R) and pcDNA3-myc-β-trophin mutant (K158R), the band was less intense than the wild type. These results suggest that less amount of ubiquitin was detected, since the ubiquitin did not bind to the mutant plasmids (FIG. 5, lanes 3, 5 and 6). These results explain that (β-trophin first binds to ubiquitin, and then poly-ubiquitin chain, and then is degraded through the polyubiquitin chain with is formed by ubiquitin-proteasome system.

3. Assessment of 3-Trophin Half-Life Using Protein Synthesis Inhibitor Cyclohexamide (CHX)

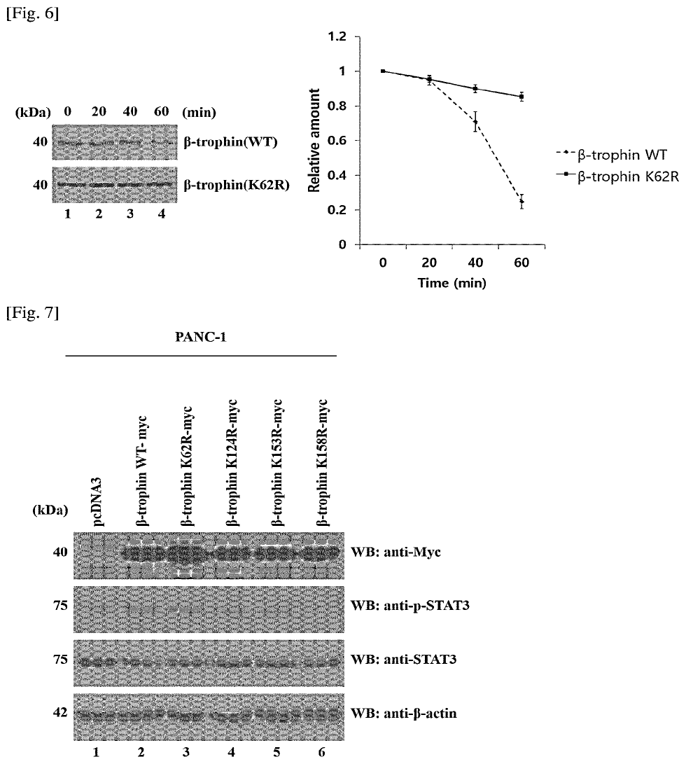

The HEK 293T cell was transfected with 2 μg of pcDNA3-myc-β-trophin WT, pcDNA3-myc-β-trophin mutant (K62R), pcDNA3-myc-β-trophin mutant (K124R), pcDNA3-myc-β-trophin mutant (K153R) and pcDNA3-myc-β-trophin mutant (K158R), respectively. 48 hrs after the transfection, the cell was treated with the protein synthesis inhibitor, cyclohexamide (CHX) (Sigma-Aldrich) (100 μg/ml), and then the half-life of each protein was detected at 20 min, 40 min and 60 min, after the treatment of the protein synthesis inhibitor. As a result, the degradation of human β-trophin was observed (FIG. 6). The half-life of human β-trophin was less than 1 hr, while the half-lives of β-trophin mutant (K62R) and β-trophin mutant (K158R) were prolonged to 1 hr or more, as shown in FIG. 6.

4. Signal Transduction by β-Trophin and the Substituted β-Trophin in Cells

It was reported that the temporarily expressed β-trophin in a mouse liver catalyzed pancreatic β cell proliferation (Cell, 153, 747-758, 2013). In this experiment, we examined the signal transduction by β-trophin and the substituted β-trophin in cells. First, the PANC-1 cell (ATCC, CRL-1469) was washed 7 times with PBS, and then transfected by using 3 μg of cDNA3-myc-β-trophin WT, pcDNA3-myc-β-trophin mutant (K62R), pcDNA3-myc-β-trophin mutant (K124R), pcDNA3-myc-β-trophin mutant (K153R) and pcDNA3-myc-β-trophin mutant (K158R), respectively. 2 days after the transfection, the proteins were extracted from the cells and quantified. Western blot was performed to analyze the signal transduction in the cells. For this purpose, the proteins separated from the PANC-1 cell transfected with respective pcDNA3-myc-β-trophin WT, pcDNA3-myc-β-trophin mutant (K62R), pcDNA3-myc-β-trophin mutant (K124R), pcDNA3-myc-β-trophin mutant (K153R) and pcDNA3-myc-β-trophin mutant (K158R) were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-myc (9E10, Santa Cruz Biotechnology, sc-40), anti-STAT3 (Santa Cruz Biotechnology, sc-21876), anti-phospho-STAT3 (Y705, cell signaling 9131S) and anti-β-actin (Santa Cruz Biotechnology, sc-47778) in 1:1,000 (w/w). As a result, pcDNA3-myc-β-trophin mutant (K62R), pcDNA3-myc-β-trophin mutant (K124R) and pcDNA3-myc-β-trophin mutant (K153R) showed the same or increased phospho-STAT3 signal transduction in the PANC-1 cell, in comparison to the wild type (FIG. 7).

### Example 2: The Analysis of Ubiquitination and Half-Life Prolonging of Growth Hormone, and the Analysis of Signal Transduction in a Cell

1. GH Expression Vector Cloning and Protein Expression

(1) GH Expression Vector Cloning

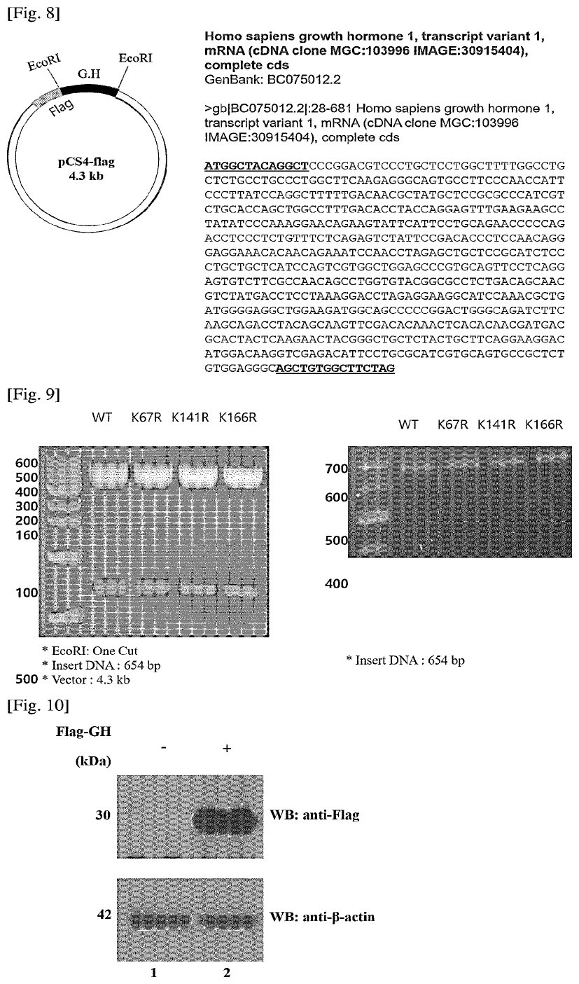

The GH DNA amplified by PCR was treated with EcoRI, and then ligated to pCS4-flag vector (4.3 kb, Oncotarget., 7(12), 14441-14457, 2016) previously digested with the same enzyme (FIG. 8, GH amino acid sequence: SEQ ID NO: 10). Then, agarose gel electrophoresis was carried out to confirm the presence of the DNA insert, after restriction enzyme digestion of the cloned vector (FIG. 9). The PCR conditions are as follows: Step 1: at 94° C. for 3 minutes (1 cycle); Step 2: at 94° C. for 30 seconds; at 60° C. for 30 seconds; at 72° C. for 30 seconds (25 cycles); and Step 3: at 72° C. for 10 minutes (1 cycle), and then held at 4° C. The nucleotide sequences in underlined bold letters in FIG. 8 indicate the primer sets used for the PCR to confirm the cloned sites (FIG. 9). For the analysis of protein expression, western blot was carried out with the use of anti-flag (Sigma-aldrich, F3165) antibody to flag of pCS4-flag vector in the map of FIG. 8. The western blot result showed that the growth hormone was expressed well. The normalization with actin assured that proper amount of protein was loaded (FIG. 10).

(2) Lysine (Lysine, K) Residue Substitution

Lysine residue was replaced with arginine (Arginine, R) using site-directed mutagenesis. The following primer sets were used for PCR to produce the substituted plasmid DNAs.

(GH K67R)
FP
(SEQ ID NO: 11)
5′-CCAAAGGAACAGAGGTATTCATTC-3′,
RP
(SEQ ID NO: 12)
5′-CAGGAATGAATACCTCTGTTCCTT-3′;
(GH K141R)
FP
(SEQ ID NO: 13)
5′-GACCTCCTAAGGGACCTAGAG-3′,
RP
(SEQ ID NO: 14)
5′-CTCTAGGTCCCTTAGGAGGTC-3′;
and
(GH K166R)
FP
(SEQ ID NO: 15)
5′-CAGATCTTCAGGCAGACCTAC-3′,
RP
(SEQ ID NO: 16)
5′-GTAGGTCTGCCTGAAGATCTG-3′.

Three mutant plasmid DNAs each of which one or more lysine residues were replaced by arginine (K→R) were produced using pcDNA3-myc-β-growth hormone as a template (Table 2).

TABLE 2

| Lysine(K) residue site | GH construct, replacement of K with R |
|---|---|
| 67 | pCS4-flag-GH (K67R) |
| 141 | pCS4-flag-GH (K141R) |
| 166 | pCS4-flag-GH (K166R) |

2. In Vivo Ubiquitination Analysis

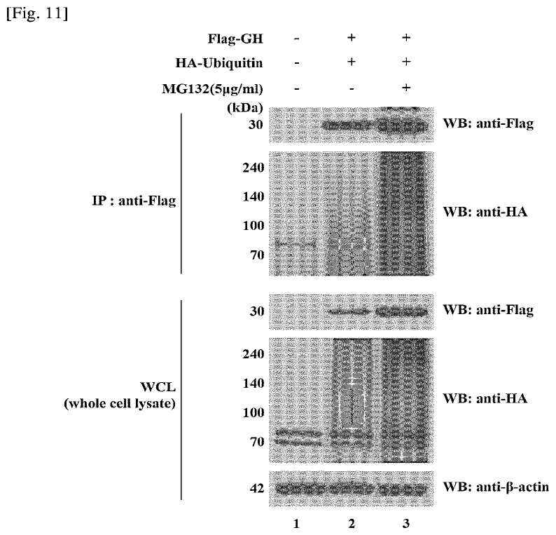

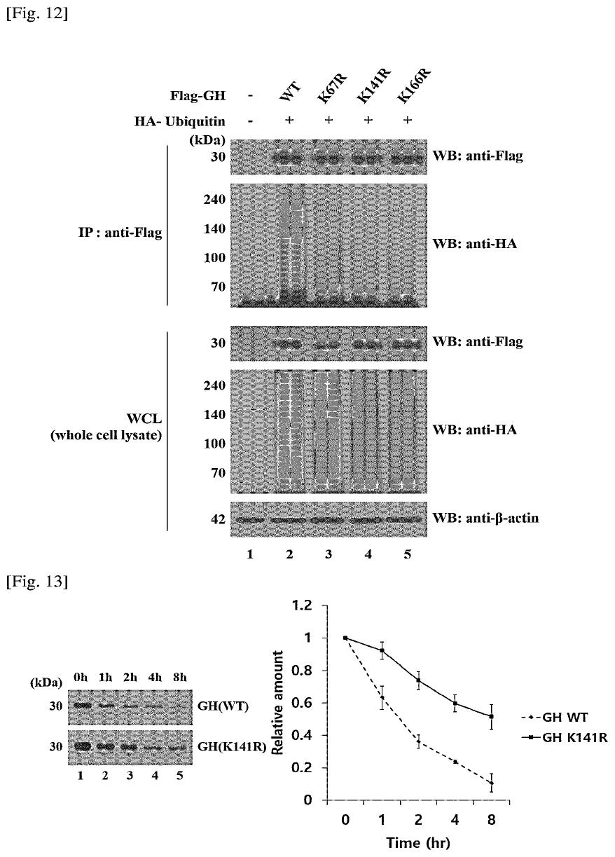

The HEK 293T cell was transfected with the plasmid encoding pCS4-flag-GH WT and pMT123-HA-ubiquitin. For the analysis of the ubiquitination level, pCS4-flag-GH WT 2 μg and pMT123-HA-ubiquitin DNA 1 μg were co-transfected into the cell. 24 hrs after the transfection, the cells were treated with MG132 (proteasome inhibitor, 5 μg/ml) for 6 hrs, thereafter immunoprecipitation analysis was carried out (FIG. 11). Then, the HEK 293T cells were transfected with the plasmids encoding pCS4-flag-GH WT, pCS4-flag-GH mutant (K67R), pCS4-flag-GH mutant (K141R), pCS4-flag-GH mutant (K166R) and pMT123-HA-ubiquitin, respectively. For the assessment of the ubiquitination level, the cells were co-transfected with 1 μg of pMT123-HA-ubiquitin DNA, and with respective 2 μg of pCS4-flag-growth hormone WT, pCS4-flag-growth hormone mutant (K67R), pCS4-flag-growth hormone mutant (K141R) and pCS4-flag-growth hormone mutant (K166R). Next, 24 hrs after the transfection, immunoprecipitation was carried out (FIG. 12). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-flag (Sigma-aldrich, F3165) 1st antibody (Santa Cruz Biotechnology, sc-40). Subsequently, the mixture was incubated at 4° C., overnight. The immunoprecipitant was separated, following the reaction with A/G bead at 4° C., for 2 hrs. Then, the separated immunoprecipitant was washed twice with buffering solution.

The protein sample was separated by SDS-PAGE, after mixing with 2×SDS buffer and heating at 100° C., for 7 minutes. The separated protein was moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-flag (Sigma-aldrich, F3165), anti-HA (sc-7392) and anti-3-actin (sc-47778) in 1:1,000 (w/w). As a result, when immunoprecipitation was performed by using anti-flag (Sigma-aldrich, F3165), poly-ubiquitin chain was formed by the binding of the ubiquitin to pCS4-flag-growth hormone WT, and thereby intense band indicating smear ubiquitin was produced (FIG. 11, lanes 2 and 3). Further, when the cells were treated with MG132 (proteasome inhibitor, 5 μg/ml) for 6 hrs, poly-ubiquitin chain formation was increased, and thus the more intense band indicating ubiquitin was shown (FIG. 11, lane 3). Further, as for the pCS4-flag-growth hormone mutant (K67R), pCS4-flag-growth hormone mutant (K141R) and pCS4-flag-growth hormone mutant (K166R), the band was less intense, in comparison to the wild type (FIG. 12, lanes 3-5). These results suggest that less amount of ubiquitin was detected since the ubiquitin did not bind to the mutant plasmids. These results explain that β-trophin first binds to ubiquitin, and then polyubiquitin chain, and then is degraded through the polyubiquitin chain with is formed by ubiquitin-proteasome system.

3. Analysis of Growth Hormone Half-Life Using Protein Synthesis Inhibitor Cyclohexamide (CHX)

The HEK 293T cell was transfected with 2 μg of pCS4-flag-growth hormone WT, pCS4-flag-growth hormone mutant (K67R), pCS4-flag-growth hormone mutant (K141R) and pCS4-flag-growth hormone mutant (K166R), respectively. 48 hrs after the transfection, the cells were treated with the protein synthesis inhibitor, cyclohexamide (CHX) (Sigma-Aldrich) (100 μg/ml), and then the half-life of each protein was detected at 1 hr, 2 hrs, 4 hrs and 8 hrs after the treatment of the said inhibitor. As a result, the degradation of human growth hormone was observed (FIG. 13). The half-life of human growth hormone was less than 2 hrs, while the half-life of pCS4-flag-growth hormone mutant (K141R) was prolonged to 8 hrs or more, as shown in FIG. 13.

4. Signal Transduction by Growth Hormone and the Substituted Growth Hormone in Cells

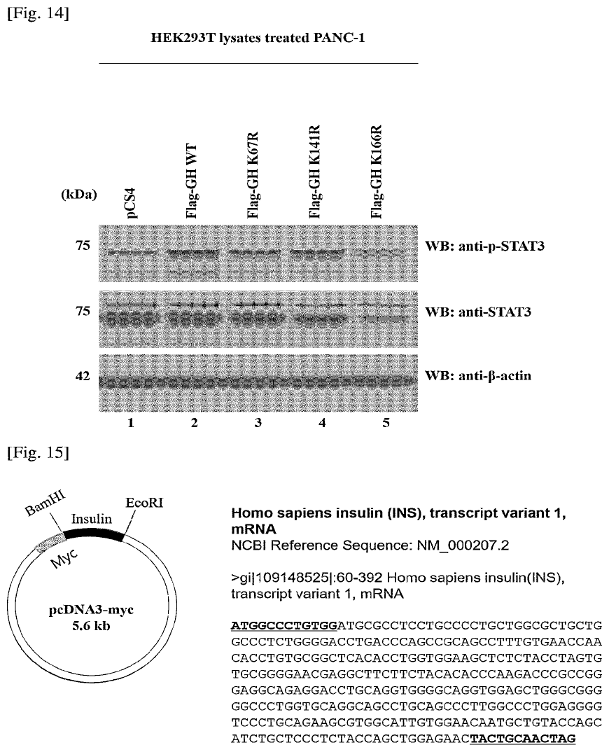

It was reported that the growth hormone controls the transcription of STAT (signal transducers and activators of transcription) protein (Oncogene, 19, 2585-2597, 2000). In this experiment, we examined the signal transduction by growth hormone and the substituted growth hormone in cells. First, the HEK 293T cell was transfected with 3 μg of pCS4-flag-growth hormone WT, pCS4-flag-growth hormone mutant (K67R), pCS4-flag-growth hormone mutant (K141R) and pCS4-flag-growth hormone mutant (K166R), respectively. 1 day after the transfection, proteins were obtained from the cells lysis by sonication. PANC-1 cell (ATCC, CRL-1469) was washed 7 times with PBS, and then transfected by using 3 μg of the obtained proteins above. Western blot was performed to analyze the signal transduction in cells. For this purpose, the proteins separated from the PANC-1 cells transfected with respective pCS4-flag-growth hormone WT, pCS4-flag-growth hormone mutant (K67R), pCS4-flag-growth hormone mutant (K141R) and pCS4-flag-growth hormone mutant (K166R), were moved to PVDF membrane. Next, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-STAT3 (sc-21876), antiphospho-STAT3 (Y705, Cell Signaling Technology, 9131S) and anti-β-actin (sc-47778) in 1:1,000 (w/w). As a result, pCS4-flag-growth hormone mutant (K141R) showed the same or increased phospho-STAT3 in the PANC-1 cell, in comparison to the pCS4-flag-growth hormone WT, and pCS4-flag-growth hormone mutant (K67R) showed increased phospho-STAT3 signal transduction in comparison with the control (FIG. 14).

### Example 3: The Analysis of Ubiquitination and Half-Life Increase of Insulin, and the Analysis of Signal Transduction in Cells

1. Insulin Expression Vector Cloning and Protein Expression

(1) Insulin Expression Vector Cloning

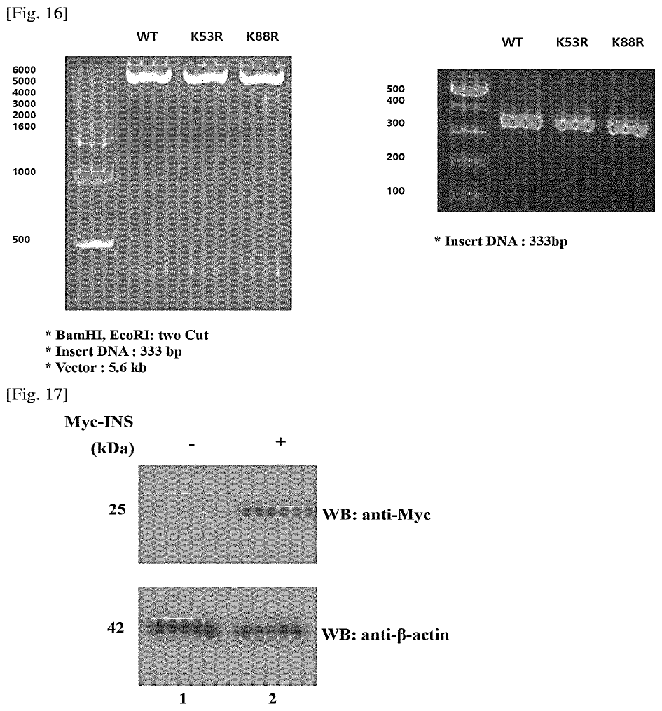

The insulin DNA amplification products by PCR was treated with BamHI and EcoRI, and then ligated to pcDNA3-myc vector (5.6 kb) previously digested with the same enzyme (FIG. 15, insulin amino acid sequence: SEQ ID NO: 17). Then, agarose gel electrophoresis was carried out to confirm the presence of the DNA insert, after restriction enzyme digestion of the cloned vector (FIG. 16). The PCR conditions are as follows: Step 1: at 94° C. for 3 minutes (1 cycle); Step 2: at 94° C. for 30 seconds; at 60° C. for 30 seconds; at 72° C. for 30 seconds (25 cycles); and Step 3: at 72° C. for 10 minutes (1 cycle), and then held at 4° C. The nucleotide sequences shown in underlined bold letters in FIG. 15 indicate the primer sets used for the PCR to confirm the cloned sites (FIG. 16). For the assessment of the expression of proteins encoded by cloned DNA, western blot was carried out with anti-myc antibody (9E10, sc-40) to myc of pcDNA3-myc vector shown in the map of FIG. 15. The western blot result showed that the insulin was expressed well. The normalization with actin assured that proper amount of protein was loaded (FIG. 17).

(2) Lysine (Lysine, K) Residue Substitution

Lysine residue was replaced by arginine (Arginine, R) using site-directed mutagenesis. The following primer sets were used for PCR to prepare the substituted plasmid DNAs.

(insulin K53R)
FP
(SEQ ID NO: 18)
5′-GGCTTCTTCTACACACCCAGGACCC-3′,
RP
(SEQ ID NO: 19)
5′-CTCCCGGCGGGTCCTGGGTGTGTA-3′;
and
(insulin K88R)
FP
(SEQ ID NO: 20)
5′-TCCCTGCAGAGGCGTGGCATTGT-3′,
RP
(SEQ ID NO: 21)
5′-TTGTTCCACAATGCCACGCCTCTGC AG-3′.

Two plasmid DNAs each of which one or more lysine residues were replaced with arginine (K→R) were produced by using pcDNA3-myc-insulin as a template (Table 3).

TABLE 3

| Lysine(K) residue site | insulin construct, replacement of K with R |
|---|---|
| 53 | pcDNA3-myc-insulin (K53R) |
| 88 | pcDNA3-myc-insulin (K88R) |

2. In Vivo Ubiquitination Analysis

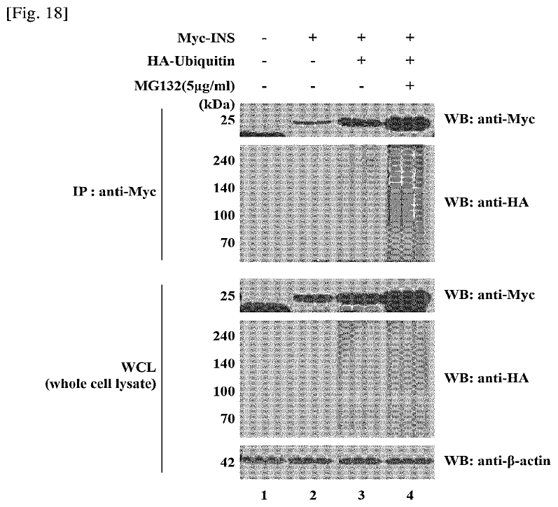

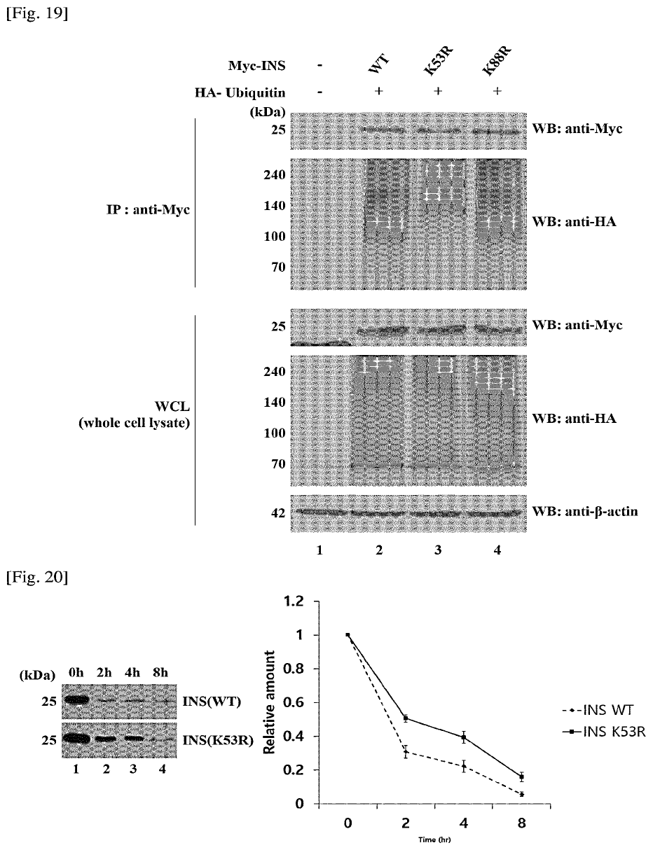

The HEK 293T cell was transfected with the plasmid encoding pcDNA3-myc-insulin WT and pMT123-HA-ubiquitin. For the analysis of the ubiquitination level, cDNA3-myc-insulin WT 2 μg and pMT123-HA-ubiquitin DNA 1 μg were co-transfected into the cells. 24 hrs after the transfection, the cells were treated with MG132 (5 μg/e) for 6 hrs, and thereafter immunoprecipitation was carried out (FIG. 18). Then, the HEK 293T cells were transfected with the plasmids encoding pcDNA3-myc-insulin WT, pcDNA3-myc-insulin mutant (K53R), pcDNA3-myc-insulin mutant (K88R) and pMT123-HA-ubiquitin, respectively. Further, for the analysis of the ubiquitination level, the cells were co-transfected with 1 μg of pMT123-HA-ubiquitin DNA, and with respective 2 μg of pcDNA3-myc-insulin WT, pcDNA3-myc-insulin mutant (K53R) and pcDNA3-myc-insulin mutant (K88R). Next, 24 hrs after the transfection, immunoprecipitation was carried out (FIG. 19). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution.

The protein sample was separated by SDS-PAGE, after mixing with 2×SDS buffer and heating ing at 100° C., for 7 min. The separated protein was moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β-actin (sc-47778) in 1:1,000 (w/w). As a result, when immunoprecipitation was performed with anti-myc (9E10, sc-40), poly-ubiquitin chain was formed by the binding of ubiquitin to pcDNA3-myc-insulin WT, and thereby intense band indicating the presence of smear ubiquitin was produced (FIG. 18, lane 3 and 4). Further, when the cells were treated with MG132 (proteasome inhibitor, 5 μg/ml) for 6 hrs, poly-ubiquitin chain formation was increased, and thus the more intense band indicating ubiquitin was shown (FIG. 18, lane 4). Further, as for the pcDNA3-myc-insulin mutant (K53R), the band was less intense than the wild type, and smaller amount of ubiquitin was detected, since the pcDNA3-myc-insulin mutant (K53R) was not bound to the ubiquitin (FIG. 19, lane 3). These results teach that insulin first binds to ubiquitin, and then is degraded through the polyubiquitination which is formed by ubiquitin-proteasome system.

3. Assessment of Insulin Half-Life Using Protein Synthesis Inhibitor Cyclohexamide (CHX)

The HEK 293T cell was transfected with 2 μg of pcDNA3-myc-insulin WT, pcDNA3-myc-insulin mutant (K53R) and pcDNA3-myc-insulin mutant (K88R), respectively. 48 hrs after the transfection, the cells were treated with the protein synthesis inhibitor, cyclohexamide (CHX) (Sigma-Aldrich) (100 μg/ml), and then the half-life of each protein was detected at 2 hrs, 4 hrs and 8 hrs after the treatment of the protein synthesis inhibitor. As a result, the degradation of human insulin was observed (FIG. 20). In consequence, the half-life of human insulin was less than 30 min, while the half-life of the human pcDNA3-myc-insulin mutant (K53R) was prolonged to 1 hr or more, as shown in FIG. 20.

4. Signal Transduction by Insulin and the Substituted Insulin in Cells

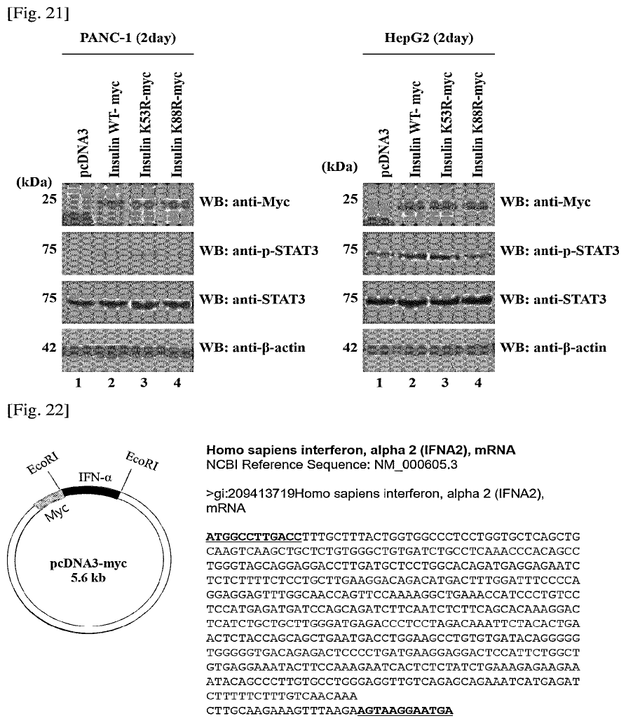

It was reported that the insulin stimulates STAT phosphorylation in liver, and thereby controls glucose homeostasis in liver (Cell Metab., 3, 267275, 2006). In this experiment, we examined the signal transduction by insulin and the substituted insulin in cells. First, the PANC-1 cell and HepG2 cell were washed 7 times with PBS, and then transfected by using 3 μg of pcDNA3-myc-insulin WT, pcDNA3-myc-insulin mutant (K53R) and pcDNA3-myc-insulin mutant (K88R), respectively. 2 days after the transfection, the proteins were extracted from the cells and quantified. Western blot was performed to analyze the signal transduction in the cells. The proteins separated from the PANC-1 and HepG2 cells transfected with respective pcDNA3-myc-insulin WT, pcDNA3-myc-insulin mutant (K53R) and pcDNA3-myc-insulin mutant (K88R), were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-STAT3 (sc-21876), anti-phospho-STAT3 (Y705, Cell Signaling 9131S) and anti-β-actin (sc-47778) in 1:1,000 (w/w). As a result, pcDNA3-myc-insulin mutant (K53R) showed the same or increased phospho-STAT3 signal transduction in PANC-1 cell and HepG2 cell, in comparison to the pcDNA3-myc-insulin WT (FIG. 21).

### Example 4: The Analysis of Ubiquitination and Half-Life Increase of Interferon-α, and the Analysis of Signal Transduction in Cells

1. Interferon-α Expression Vector Cloning and Protein Expression

(1) Interferon-α Expression Vector Cloning

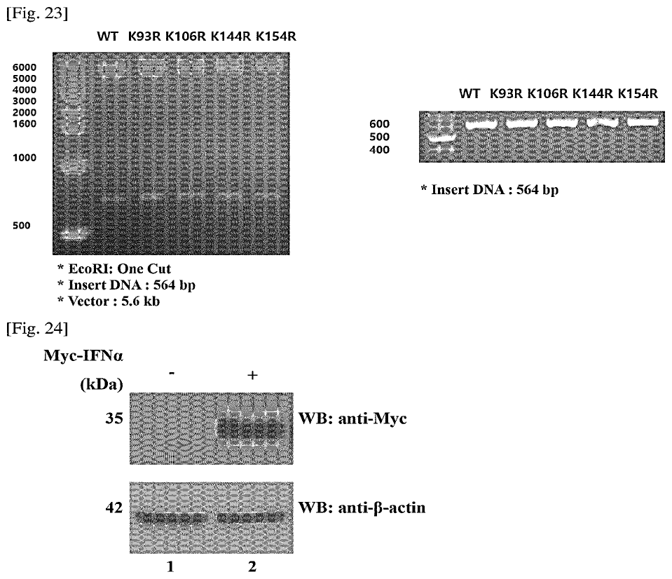

The interferon-α DNA amplified by PCR was treated with EcoRI, and then ligated to pcDNA3-myc vector (5.6 kb) previously digested with the same enzyme (FIG. 22, interferon-α amino acid sequence: SEQ ID NO: 22). Then, agarose gel electrophoresis was carried out to confirm the presence of the DNA insert, after restriction enzyme digestion of the cloned vector (FIG. 23). The nucleotide sequences shown in underlined bold letters in FIG. 22 indicate the primer sets used for the PCR to confirm the cloned sites (FIG. 23). The PCR conditions are as follows, Step 1: at 94° C. for 3 minutes (1 cycle); Step 2: at 94° C. for 30 seconds; at 58° C. for 30 seconds; at 72° C. for 1 minute (25 cycles); and Step 3: at 72° C. for 10 minutes (1 cycles), and then held at 4° C. For the assessment of the expression of proteins encoded by cloned DNA, western blot was carried out with anti-myc antibody (9E10, sc-40) to myc of pcDNA3-myc vector shown in the map of FIG. 22. The western blot results showed that the interferon-α protein bound to myc was expressed well. The normalization with actin assured that proper amount of protein was loaded (FIG. 24).

(2) Lysine (Lysine, K) Residue Substitution

Lysine residue was replaced with arginine (Arginine, R) using site-directed mutagenesis. The following primer sets were used for PCR to prepare the substituted plasmid DNAs.

(IFN-α K93R)
FP
(SEQ ID NO: 23)
5′-CTTCAGCACAAGGGACTCATC-3′,
RP
(SEQ ID NO: 24)
5′-CAGATGAGTCCCTTGTGCTGA-3′;
(IFN-α K106R)
FP
(SEQ ID NO: 25)
5′-CTCCTAGACAGATTCTACACT-3′,
RP
(SEQ ID NO: 26)
5′-AGTGTAGAATCTGTCTAGGAG-3′;
(IFN-α K144R)
FP
(SEQ ID NO: 27)
5′-GCTGTGAGGAGATACTTCCAA-3′,
RP
(SEQ ID NO: 28)
5′-TTGGAAGTATCTCCTCACAGC-3′;
and
(IFN-α K154R)
P
(SEQ ID NO: 29)
5′-CTCTATCTGAGAGAGAAGAAA-3′,
RP
(SEQ ID NO: 30))
5′-TTTCTTCTCTCTCAGATAGAG-3′.

Four plasmid DNAs each of which one or more lysine residues were replaced by arginine (K→R) were prepared by using pcDNA3-myc-interferon-α as a template (Table 4).

TABLE 4

| Lysine(K) residue site | interferon-α construct, replacement of K with R |
|---|---|
| 93 | pcDNA3-myc-IFN-α (K93R) |
| 106 | pcDNA3-myc-IFN-α (K106R) |
| 144 | pcDNA3-myc-IFN-α (K144R) |
| 154 | pcDNA3-myc-IFN-α (K154R) |

2. In Vivo Ubiquitination Analysis

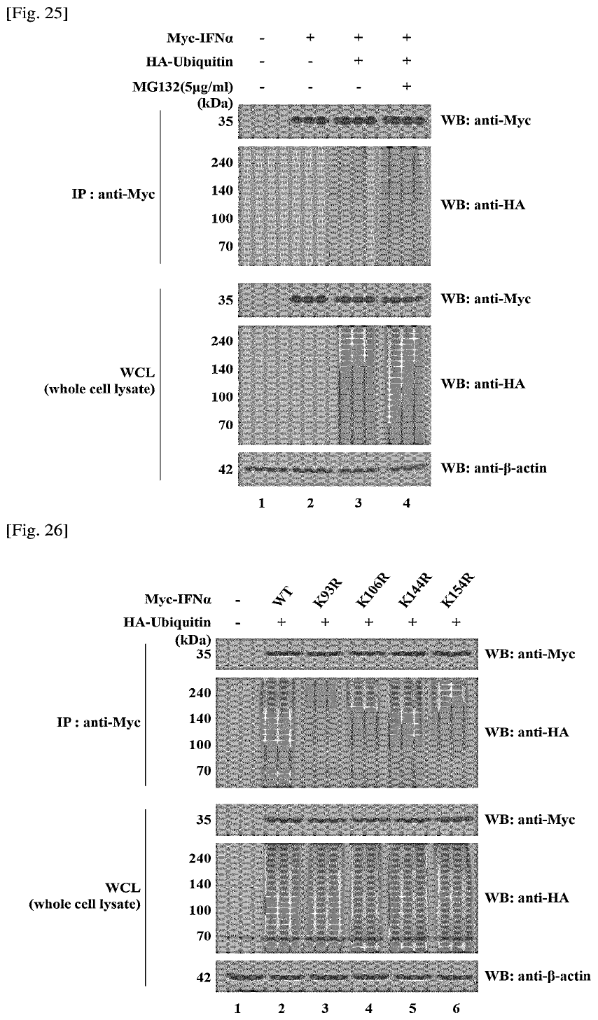

The HEK 293T cell was transfected with the plasmid encoding pcDNA3-myc-interferon-α WT and pMT123-HA-ubiquitin. For the analysis of the ubiquitination level, pcDNA3-myc-interferon-α WT 2 μg and pMT123-HA-ubiquitin DNA 1 μg were co-transfected into the cells. 24 hrs after the transfection, the cells were treated with MG132 (proteasome inhibitor, 5 μg/ml) for 6 hrs, thereafter immunoprecipitation analysis was carried out (FIG. 25). Then, the HEK 293T cells were transfected with the plasmids encoding pcDNA3-myc-interferon-α WT, pcDNA3-myc-interferon-α mutant (K93R), pcDNA3-myc-interferon-α mutant (K106R), pcDNA3-myc-interferon-α mutant (K144R), pcDNA3-myc-interferon-α mutant (K154R) and pMT123-HA-ubiquitin, respectively. For the analysis of the ubiquitination level, the cells were co-transfected with 1 μg of pMT123-HA-ubiquitin DNA, and with respective 2 μg of pcDNA3-myc-interferon-α WT, pcDNA3-myc-interferon-α mutant (K93R), pcDNA3-myc-interferon-α mutant (K106R), pcDNA3-myc-interferon-α mutant (K144R) and pcDNA3-myc-interferon-α mutant (K154R). Next, 24 hrs after the transfection, immunoprecipitation was carried out (FIG. 26). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution.

The protein sample was separated by SDS-PAGE, after mixing with 2×SDS buffer and heating at 100° C., for 7 minutes. The separated proteins were moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), poly-ubiquitin chain was produced by the binding of the ubiquitin to pcDNA3-myc-interferon-α WT, and thereby intense band indicating the presence of smear ubiquitin was detected (FIG. 25, lanes 3 and 4). Further, when the cells were treated with MG132 (proteasome inhibitor, 5 μg/ml) for 6 hrs, poly-ubiquitin chain formation was increased, and thus the more intense band indicating ubiquitin was produced (FIG. 25, lane 4). Further, as for the pcDNA3-myc-interferon-α mutant (K93R), pcDNA3-myc-interferon-α mutant (K106R), pcDNA3-myc-interferon-α mutant (K144R) and pcDNA3-myc-interferon-α mutant (K154R), the band was less intense than the wild type, and smaller amount of ubiquitin was detected since the mutant plasmids were not bound to the ubiquitin (FIG. 26, lanes 3 to 6). These results explain that interferon-α first binds to ubiquitin, and then is degraded through the polyubiquitin chain which is formed by ubiquitin-proteasome system.

3. Assessment of Interferon-α Half-Life Using Protein Synthesis Inhibitor Cyclohexamide (CHX)

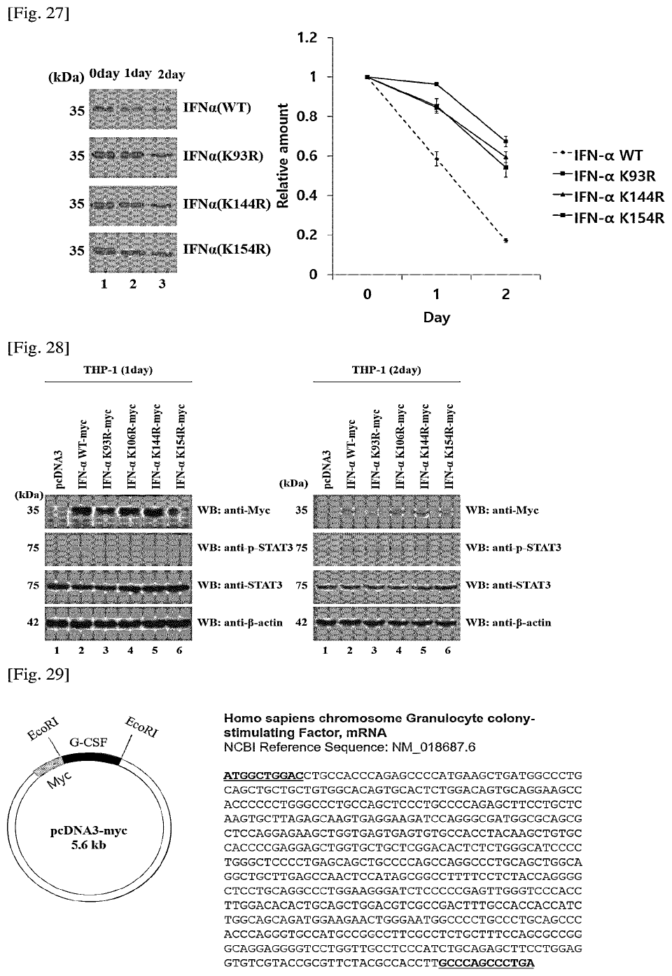

The HEK 293T cell was transfected with respective 2 μg of pcDNA3-myc-interferon-α mutant WT, pcDNA3-myc-interferon-α mutant (K93R), pcDNA3-myc-interferon-α mutant (K106R), pcDNA3-myc-interferon-α mutant (K144R) and pcDNA3-myc-interferon-α mutant (K154R), respectively. 48 hrs after the transfection, the cells were treated with the protein synthesis inhibitor, cyclohexamide (CHX) (Sigma-Aldrich) (100 μg/ml), and then the half-life of each protein was detected for 1 day and 2 days after the treatment of the protein synthesis inhibitor. As a result, the degradation of human interferon-α was observed (FIG. 27). The half-life of human interferon-α was less than 1 day, while the half-lives of pcDNA3-myc-interferon-α mutant (K93R), pcDNA3-myc-interferon-α mutant (K144R) and pcDNA3-myc-interferon-α mutant (K154R) were prolonged to 2 days or more, as shown in FIG. 27.

4. Signal Transduction by Interferon-α and the Substituted Interferon-α in Cells

It was reported that the IFN-α enhances STAT-1, STAT-2 and STAT-3 (J Immunol., 187, 2578-2585, 2011), and the IFN-α activates the STAT3 protein which contributes to melanoma tumorigenesis (Eur J Cancer, 45, 1315-1323, 2009). In this experiment, we examined the signal transduction by interferon-α and the substituted interferon-α in cells. First, THP-1 cell (ATCC, TIB-202) was washed 7 times with PBS, and then transfected by using 3 μg of pcDNA3-myc-interferon-α WT, pcDNA3-myc-interferon-α mutant (K93R), pcDNA3-myc-interferon-α mutant (K106R), pcDNA3-myc-interferon-α mutant (K144R) and pcDNA3-myc-interferon-α mutant (K154R), respectively. 1 day and 2 days after the transfection, the proteins were extracted from the cells and quantified. Western blot was performed to analyze the signal transduction in the cells. The proteins separated from the THP-1 cell transfected with respective pcDNA3-myc-interferon-α WT, pcDNA3-myc-interferon-α mutant (K93R), pcDNA3-myc-interferon-α mutant (K106R), pcDNA3-myc-interferon-α mutant (K144R) and pcDNA3-myc-interferon-α mutant (K154R) were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-STAT3 (sc-21876), anti-phospho-STAT3 (Y705, cell signaling 9131S) and anti-β-actin (sc-47778) in 1:1,000 (w/w). As a result, pcDNA3-myc-interferon-α mutant (K93R), pcDNA3-myc-interferon-α mutant (K106R), pcDNA3-myc-interferon-α mutant (K144R) and pcDNA3-myc-interferon-α mutant (K154R) showed the same or increased phospho-STAT3 signal transduction in THP-1 cell, in comparison to the pcDNA3-myc-interferon-α WT (FIG. 28)

### Example 5: The Analysis of Ubiquitination and Half-Life Increase of G-CSF, and the Analysis of Signal Transduction in Cells

1. G-CSF Expression Vector Cloning and Protein Expression

(1) G-CSF Expression Vector Cloning

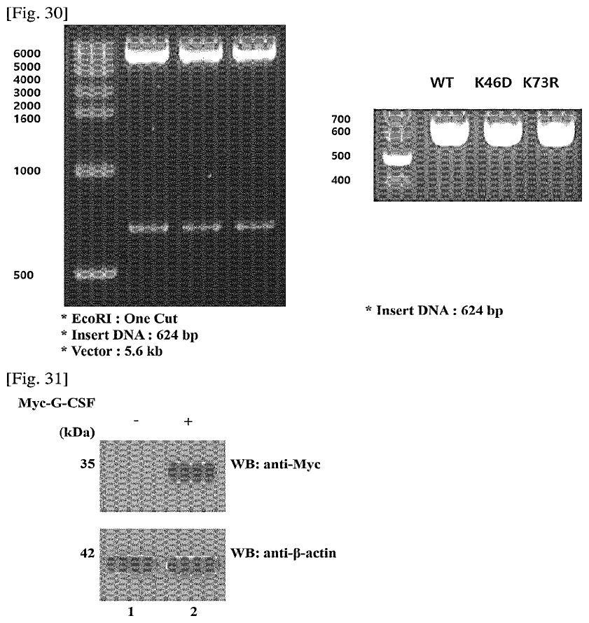

The G-CSF DNA amplified by PCR was treated with EcoRI, and then ligated to pcDNA3-myc vector (5.6 kb) previously digested with the same enzyme (FIG. 29, G-CSF amino acid sequence: SEQ ID NO: 31). Then, agarose gel electrophoresis was carried out to confirm the presence of the DNA insert, after restriction enzyme digestion of the cloned vector (FIG. 30). The nucleotide sequences shown in underlined bold letters in FIG. 29 indicate the primer sets used for the PCR to confirm the cloned sites (FIG. 30). The PCR conditions are as follows, Step 1: at 94° C. for 3 minutes (1 cycle); Step 2: at 94° C. for 30 seconds; at 58° C. for 30 seconds; at 72° C. for 1 minute (25 cycles); and Step 3: at 72° C. for 10 minutes (1 cycle), and then held at 4° C. For the assessment of the expression of proteins encoded by cloned DNA, western blot was carried out with anti-myc antibody (9E10, sc-40) to myc of pcDNA3-myc vector shown in the map of FIG. 29. The western blot result showed that the G-CSF protein bound to myc was expressed well. The normalization with actin assured that proper amount of protein was loaded (FIG. 31).

(2) Lysine (Lysine, K) Residue Substitution

Lysine residue was replaced with arginine (Arginine, R) using site-directed mutagenesis. The following primer sets were used for PCR to prepare the substituted plasmid DNAs.

(G-CSF K46R)
FP
(SEQ ID NO: 32)
5′-AGCTTCCTGCTCAGGTGCTTAGAG-3′,
RP
(SEQ ID NO: 33)
5′-TTGCTCTAAGCACCTGAGCAGGAA-3′;
and
(G-CSF K73R)
FP
(SEQ ID NO: 34)
5′-TGTGCCACCTACAGGCTGTGCCAC-3′,
RP
(SEQ ID NO: 35)
5′-GGGGTGGCACAGCCTGTAGGTGGC-3′.

Two plasmid DNAs each of which one or more lysine residues were replaced by arginine (K→R) were prepared by using pcDNA3-myc-G-CSF as a template (Table 5).

TABLE 5

| Lysine(K) residue site | G-CSF construct, replacement of K with R |
|---|---|
| 46 | pcDNA3-myc-G-CSF (K46R) |
| 73 | pcDNA3-myc-G-CSF (K73R) |

2. In Vivo Ubiquitination Analysis

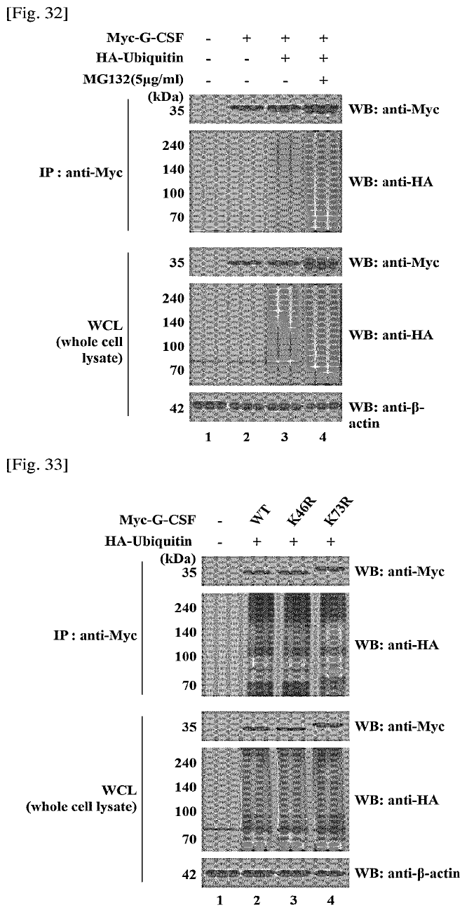

The HEK 293T cell (ATCC, CRL-3216) was transfected with the plasmid encoding pcDNA3-myc-G-CSF WT and pMT123-HA-ubiquitin. For the analysis of the ubiquitination level, pcDNA3-myc-G-CSF WT 2 μg and pMT123-HA-ubiquitin DNA 1 μg were co-transfected into the cell. 24 hrs after the transfection, the cell was treated with MG132 (proteasome inhibitor, 5 μg/ml) for 6 hrs, thereafter immunoprecipitation analysis was carried out (FIG. 32). Then, the HEK 293T cells were transfected with the plasmids encoding pcDNA3-myc-GCSF WT, pcDNA3-myc-G-CSF mutant (K46R), pcDNA3-myc-G-CSF (K73R) and pMT123-HA-ubiquitin, respectively. For the analysis of the ubiquitination level, the cells were co-transfected with 1 μg of pMT123-HA-ubiquitin DNA, and respective 2 μg of pcDNA3-myc-G-CSF WT, pcDNA3-myc-G-CSF mutant (K46R) and pcDNA3-myc-G-CSF (K73R). Next, 24 hrs after the transfection, the immunoprecipitation was carried out (FIG. 33). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C. overnight. The immunoprecipitant was separated, following the reaction with A/G bead (Santa Cruz Biotechnology) at 4° C., for 2 hrs. Subsequently, the separated immunoprecipitant was washed twice with buffering solution.

The protein sample was separated by SDS-PAGE, after mixing with 2×SDS buffer and heating at 100° C., for 7 minutes. The separated proteins were moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), poly-ubiquitin chain was formed by the binding of the ubiquitin to pcDNA3-myc-G-CSF WT, and thereby intense band indicating the presence of smear ubiquitin was detected (FIG. 32, lanes 3 and 4). Further, when the cells were treated with MG132 (proteasome inhibitor, 5 μg/ml) for 6 hrs, poly-ubiquitin chain formation was increased, and thus the more intense band indicating ubiquitin was produced (FIG. 32, lane 4). Further, as for the pcDNA3-myc-G-CSF (K73R), the band was less intense than the wild type, and smaller amount of ubiquitin was detected since pcDNA3-myc-G-CSF mutant (K73R) was not bound to the ubiquitin (FIG. 33, lane 4). These results show that G-CSF first binds to ubiquitin, and then is degraded through the polyubiquitination which is formed by ubiquitin-proteasome system.

3. Assessment of G-CSF Half-Life Using Protein Synthesis Inhibitor Cyclohexamide (CHX)

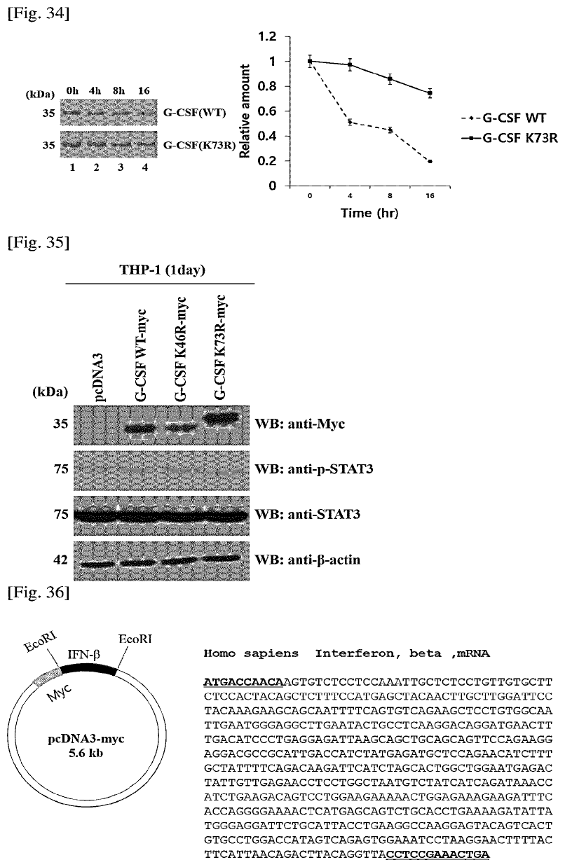

The HEK 293T cell was transfected with 2 μg of pcDNA3-myc-G-CSF WT, pcDNA3-myc-G-CSF mutant (K46R) and pcDNA3-myc-G-CSF (K73R), respectively. 48 hrs after the transfection, the cells were treated with the protein synthesis inhibitor, cyclohexamide (CHX) (Sigma-Aldrich) (100 μg/ml), and then the half-life of each protein was detected at 4 hrs, 8 hrs and 16 hrs after the treatment of the protein synthesis inhibitor. As a result, the degradation of human G-CSF was observed (FIG. 34). The half-life of human G-CSF was less than about 4 hr, while the half-life of the substituted human G-CSF (K73R) was prolonged to 16 hrs or more, as shown in FIG. 34.

4. Signal Transduction by G-CSF and the Substituted G-CSF in Cells

It was reported that the G-CSF activates STAT3 in glioma cells, and thereby is involved in glioma growth (Cancer Biol Ther., 13(6), 389-400, 2012). Further, it was reported that the G-CSF is expressed in ovarian epithelial cancer cells and is pathologically related to women uterine carcinoma by regulating JAK2/STAT3 pathway (Br J Cancer, 110, 133-145, 2014). In this experiment, we examined the signal transduction by G-CSF and the substituted G-CSF in cells. First, the THP-1 cell (ATCC, TIB-202) was washed 7 times with PBS, and then transfected by using 3 μg of pcDNA3-myc-G-CSF WT, pcDNA3-myc-G-CSF mutant (K46R) and pcDNA3-myc-G-CSF mutant (K73R), respectively. 1 day after the transfection, the proteins were extracted from the cells and quantified. Western blot was performed to analyze the signal transduction in the cells. The proteins separated from the THP-1 cell transfected with respective pcDNA3-myc-G-CSF WT, pcDNA3-myc-G-CSF mutant (K46R) and pcDNA3-myc-G-CSF mutant (K73R), were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-STAT3 (sc-21876), anti-phospho-STAT3 (Y705, cell signaling 9131S) and anti-β-actin (sc-47778) in 1:1,000 (w/w). As a result, pcDNA3-myc-G-CSF mutant (K46R) and pcDNA3-myc-G-CSF mutant (K73R) showed the same or increased phospho-STAT3 signal transduction in THP-1 cell, in comparison to the wild type (FIG. 35).

### Example 6: The Analysis of Ubiquitination and Half-Life Increase of Interferon-β, and the Analysis of Signal Transduction in Cells

1. Interferon-β Expression Vector Cloning and Protein Expression

(1) Interferon-β Expression Vector Cloning

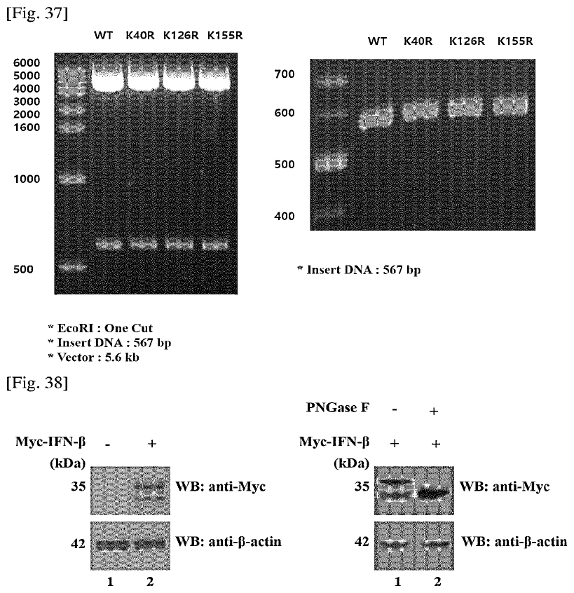

The interferon-β DNA amplified by PCR was treated with EcoRI, and then ligated to pcDNA3-myc vector (5.6 kb) previously digested with the same enzyme (FIG. 36, interferon-β amino acid sequence: SEQ ID NO: 36). Then, agarose gel electrophoresis was carried out to confirm the presence of the DNA insert, after restriction enzyme digestion of the cloned vector (FIG. 37). The nucleotide sequences shown in underlined bold letters in FIG. 36 indicate the primer sets used for the PCR to confirm the cloned sites (FIG. 37). The PCR conditions are as follows, Step 1: at 94° C. for 3 minutes (1 cycle); Step 2: at 94° C. for 30 seconds; at 58° C. for 30 seconds; at 72° C. for 50 seconds (25 cycles); and Step 3: at 72° C. for 10 minutes (1 cycle), and then held at 4° C. For the assessment of the expression of proteins encoded by cloned DNA, western blot was carried out with anti-myc antibody (9E10, sc-40) to myc of pcDNA3-myc vector shown in the map of FIG. 36. The western blot result showed that the interferon-β bound to myc was expressed well. The normalization with actin assured that proper amount of protein was loaded (FIG. 38). Further, as for the interferon-β, two kinds of expression bands were produced in the cells by glycosylation. After the treating the cells with 500 unit PNGase F (New England Biolabs Inc., P0704S), which blocks the pathway, only one band was detected (FIG. 38).

(2) Lysine (Lysine, K) Residue Substitution

Lysine residue was replaced by arginine (Arginine, R) using site-directed mutagenesis. The following primer sets were used for PCR to prepare the substituted plasmid DNAs.

(IFN-β K40R)
FP
(SEQ ID NO: 37)
5′-CAGTGTCAGAGGCTCCTGTGG-3′,
RP
(SEQ ID NO: 38)
5′-CCACAGGAGCCTCTGACACTG-3′;
(IFN-β K126R)
FP
(SEQ ID NO: 39)
5′-CTGGAAGAAAGACTGGAGAAA-3′,
RP
(SEQ ID NO: 40)
5′-TTTCTCCAGTCTTTCTTCCAG-3′;
and
(IFN-β K155R)
FP
(SEQ ID NO: 41)
5′-CATTACCTGAGGGCCAAGGAG-3′,
RP
(SEQ ID NO: 42)
5′-CTCCTTGGCCCTCAGGTAATG-3′.

Three plasmid DNAs each of which one or more lysine residues were replaced by arginine (K→R) were produced using pcDNA3-myc-interferon-β as a template (Table 6).

TABLE 6

| Lysine(K) residue site | interferon-β construct, replacement of K with R |
|---|---|
| 40 | pcDNA3-myc-IFN-β (K40R) |
| 126 | pcDNA3-myc-IFN-β (K126R) |
| 155 | pcDNA3-myc-IFN-β (K155R) |

2. In Vivo Ubiquitination Analysis

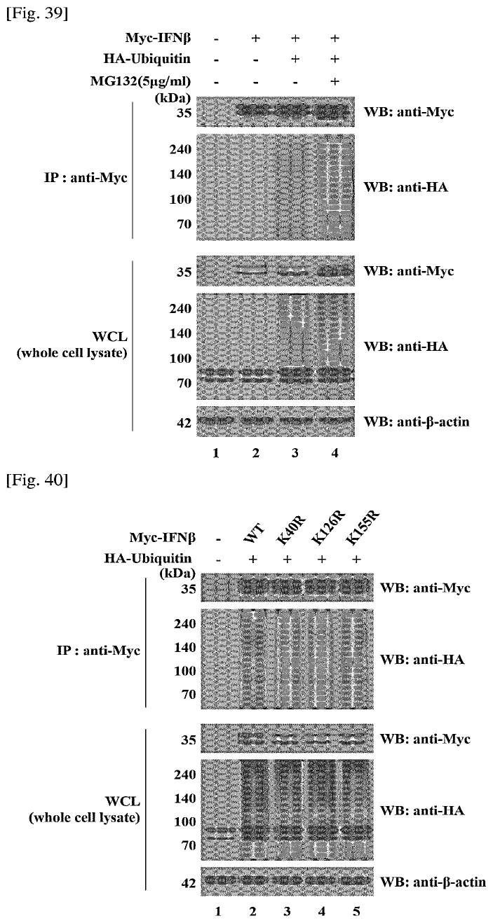

The HEK 293T cell was transfected with the plasmid encoding pcDNA3-myc-interferon-β WT and pMT123-HA-ubiquitin. For the analysis of the ubiquitination level, pcDNA3-myc-interferon-β WT 2 μg and pMT123-HA-ubiquitin DNA 1 μg were co-transfected into the cell. 24 hrs after the transfection, the cells were treated with MG132 (proteasome inhibitor, 5 μg/ml) for 6 hrs, thereafter immunoprecipitation analysis was carried out (FIG. 39). Further, the HEK 293T cells were transfected with the plasmids encoding pcDNA3-myc-interferon-3 WT, pcDNA3-myc-interferon-β mutant (K40R), pcDNA3-myc-interferon-β mutant (K126R), pcDNA3-myc-interferon-β mutant (K155R) and pMT123-HA-ubiquitin, respectively. For the analysis of the ubiquitination level, the cells were co-transfected with 1 μg of pMT123-HA-ubiquitin DNA, and respective 2 μg of pcDNA3-myc-interferon-β WT, pcDNA3-myc-interferon-β mutant (K40R), pcDNA3-myc-interferon-β mutant (K126R) and pcDNA3-myc-interferon-β mutant (K155R). Next, 24 hrs after the transfection, immunoprecipitation was carried out (FIG. 40). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution. The protein sample was separated by SDS-PAGE, after mixing with 2×SDS buffer and heating at 100° C. for 7 minutes. The separated proteins were moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β (sc-47778) in 1:1,000 (w/w). As a result, when immunoprecipitation was performed by using anti-myc (9E10, sc-40), poly-ubiquitination was formed by the binding of the ubiquitin to pcDNA3-myc-interferon-β WT, and thereby intense band indicating the presence of smear ubiquitin was detected (FIG. 39, lanes 3 and 4). Further, when the cells were treated with MG132 (proteasome inhibitor, 5 μg/ml) for 6 hrs, poly-ubiquitin chain formation was increased, and thus the more intense band indicating ubiquitin was appeared (FIG. 39, lane 4). Further, as for the pcDNA3-myc-interferon-β mutant (K40R), pcDNA3-myc-interferon-β mutant (K126R) and pcDNA3-myc-interferon-β mutant (K155R), the band was less intense than the wild type, and smaller amount of ubiquitin was detected since the mutant plasmids were not bound to the ubiquitin (FIG. 40, lanes 3 to 5). These results show that interferon-β first binds to ubiquitin, and then is degraded through the polyubiquitination which is formed by ubiquitin-proteasome system.

3. Assessment of Interferon-β Half-Life Using Protein Synthesis Inhibitor Cyclohexamide (CHX)

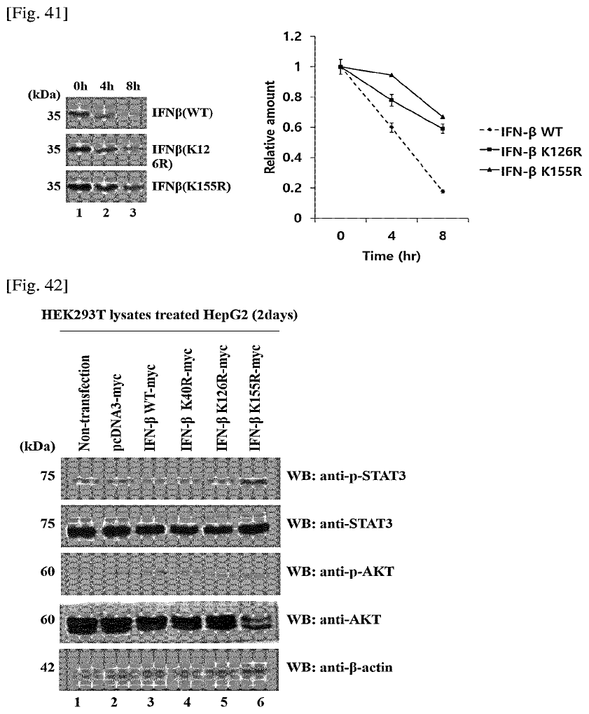

The HEK 293T cell was transfected with 2 μg of pcDNA3-myc-interferon-β WT, pcDNA3-myc-interferon-β mutant (K40R), pcDNA3-myc-interferon-β mutant (K126R) and pcDNA3-myc-interferon-β mutant (K155R), respectively. 48 hrs after the transfection, the cells were treated with the protein synthesis inhibitor, cyclohexamide (CHX) (Sigma-Aldrich) (100 μg/ml), and then the half-life of each proteins was detected at 4 hrs and 8 hrs after the treatment of the inhibitor. As a result, the degradation of human interferon-β was observed (FIG. 41). The half-life of human interferon-β was less than 4 hrs, while the half-lives of pcDNA3-myc-interferon-β mutant (K126R) and pcDNA3-myc-interferon-β mutant (K155R) were prolonged to 8 hr or more, as shown in FIG. 41.

4. Signal Transduction by Interferon-β and the Substituted Interferon-β in Cells

It was reported that the activation of signal pathways including AKT is induced by the IFN-β treated cell (Pharmaceuticals (Basel), 3, 994-1015, 2010). In this experiment, we examined the signal transduction by interferon-β and the substituted interferon-β in cells. First, HepG2 cell was starved for 8 hrs, and then transfected by using 3 μg of pcDNA3-myc-interferon-β WT, pcDNA3-myc-interferon-β mutant (K40R), pcDNA3-myc-interferon-β mutant (K126R) and pcDNA3-myc-interferon-β mutant (K155R), respectively. 1 day after the transfection, the proteins were obtained from the HepG2 cell lysis by sonication, and then the proteins were transfected into the HepG2 cells washed 7 times with PBS. 2 days after the transfection, the proteins were extracted from the cells and quantified. Western blot was performed to analyze the signal transduction in a cell. The proteins separated from the HepG2 cell transfected with respective pcDNA3-myc-interferon-β WT, pcDNA3-myc-interferon-β mutant (K40R), pcDNA3-myc-interferon-β mutant (K126R) and pcDNA3-myc-interferon-β mutant (K155R), were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-STAT3 (sc-21876), anti-phospho-STAT3 (Y705, cell signaling 9131S), anti-AKT (H-136, sc-8312), anti-phospho-AKT (S473, cell signaling 92715) and anti-β-actin (sc-47778) in 1:1,000 (w/w). As a result, pcDNA3-myc-interferon-β mutant (K40R), pcDNA3-myc-interferon-β mutant (K126R) and pcDNA3-myc-interferon-β mutant (K155R) showed the same or increased phospho-AKT signal transduction in HepG2 cell (ATCC, AB-8065), in comparison to the wild type (FIG. 42)

### Example 7: The Analysis of Ubiquitination and Half-Life Increase of Erythropoietin (EPO), and the Analysis of Signal Transduction in Cells

1. Erythropoietin (EPO) Expression Vector Cloning and Protein Expression

(1) Erythropoietin (EPO) Expression Vector Cloning

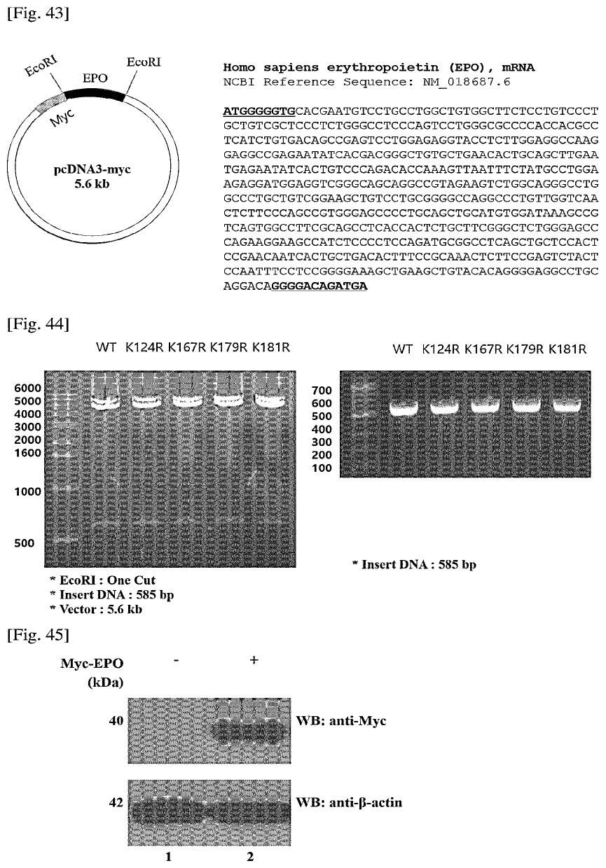

The erythropoietin (EPO) DNA amplified by PCR was treated with EcoRI, and then ligated to pcDNA3-myc vector (5.6 kb) previously digested with the same enzyme (FIG. 43, erythropoietin amino acid sequence: SEQ ID NO: 43). Then, agarose gel electrophoresis was carried out to confirm the presence of the DNA insert, after restriction enzyme digestion of the cloned vector (FIG. 44). The nucleotide sequences shown in underlined bold letters in FIG. 43 indicate the primer sets used for the PCR to confirm the cloned sites (FIG. 44). The PCR conditions are as follows, Step 1: at 94° C. for 3 minutes (1 cycle); Step 2: at 94° C. for 30 seconds; at 58° C. for 30 seconds; at 72° C. for 1 minute (25 cycles); and Step 3: at 72° C. for 10 minutes (1 cycle), and then held at 4° C. For the assessment of the expression of proteins encoded by cloned DNA, western blot was carried out with anti-myc antibody (9E10, sc-40) to myc of pcDNA3-myc vector shown in the map of FIG. 43. The western blot result showed that the EPO protein bound to myc was expressed well. The normalization with actin assured that proper amount of protein was loaded (FIG. 45).

(2) Lysine (Lysine, K) Residue Substitution

Lysine residue was replaced with arginine (Arginine, R) using site-directed mutagenesis. The following primer sets were used for PCR to prepare the substituted plasmid DNAs.

(EPO K124R)
FP
(SEQ ID NO: 44)
5′-GCATGTGGATAGAGCCGTCAGTGC-3′,
RP
(SEQ ID NO: 45)
5′-GCACTGACGGCTCTATCCACATGC-3′;
(EPO K167R)
FP
(SEQ ID NO: 46)
5′-TGACACTTTCCGCAGACTCTTCCGAGTCTAC-3′,
RP
(SEQ ID NO: 47)
5′-GTAGACTCGGAAGAGTCTGCGGAAAGTGTCA-3′;
(EPO K179R)
FP
(SEQ ID NO: 48)
5′-CTCCGGGGAAGGCTGAAGCTG-3′,
RP
(SEQ ID NO: 49)
5′-CAGCTTCAGCCTTCCC CGGAG-3′;
and
(EPO K181R)
FP
(SEQ ID NO: 50)
5′-GGAAAGCTGAGGCTGTACACAGG-3′,
RP
(SEQ ID NO: 51)
5′-CCTGTGTACAGCCTCAGCTTTCC-3′.

Four plasmid DNAs each of one or more which lysine residues were replaced by arginine (K→R) were produced by using pcDNA3-myc-EPO as a template (Table 7).

TABLE 7

| Lysine(K) residue site | β-trophin construct, replacement of K with R |
|---|---|
| 124 | pcDNA3-myc-EPO (K124R) |
| 167 | pcDNA3-myc-EPO (K167R) |
| 179 | pcDNA3-myc-EPO (K179R) |
| 181 | pcDNA3-myc-EPO (K181R) |

2. In Vivo Ubiquitination Analysis

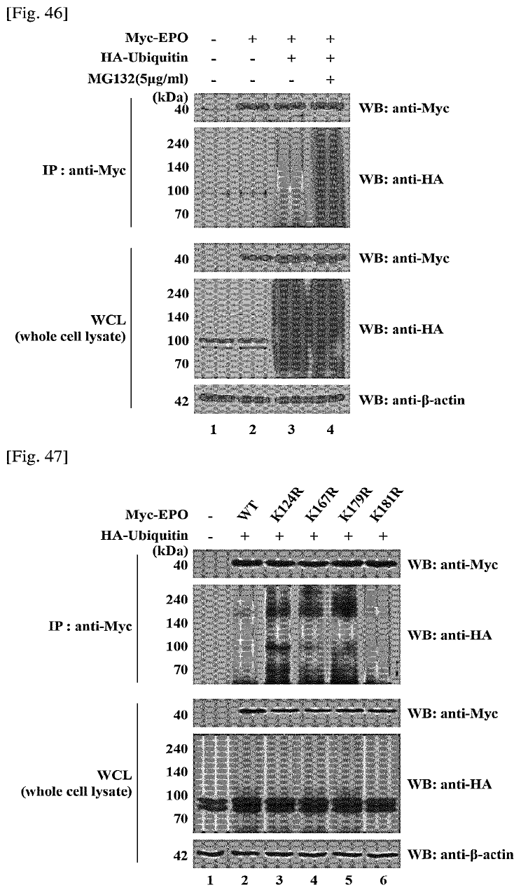

The HEK 293T cell (ATCC, CRL-3216) was transfected with the plasmid encoding pcDNA3-myc-EPO WT and pMT123-HA-ubiquitin. For the analysis of the ubiquitination level, pcDNA3-myc-EPO WT 2 μg and pMT123-HA-ubiquitin DNA 1 μg were co-transfected into the cells. 24 hrs after the transfection, the cells were treated with MG132 (proteasome inhibitor, 5 μg/ml) for 6 hrs, thereafter immunoprecipitation analysis was carried out (FIG. 46). Then, the HEK 293T cells were transfected with the plasmids encoding pcDNA3-myc-EPO WT, pcDNA3-myc-EPO mutant (K124R), pcDNA3-myc-EPO mutant (K167R), pcDNA3-myc-EPO mutant (K179R), pcDNA3-myc-EPO mutant (K181R) and pMT123-HA-ubiquitin, respectively. For the analysis of the ubiquitination level, the cells were co-transfected with 1 μg of pMT123-HA-ubiquitin DNA, and with respective 2 μg of pcDNA3-myc-EPO WT, pcDNA3-myc-EPO mutant (K124R), pcDNA3-myc-EPO mutant (K167R), pcDNA3-myc-EPO mutant (K179R) and pcDNA3-myc-EPO mutant (K181R). Next, 24 hrs after the transfection, immunoprecipitation was carried out (FIG. 47).

The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution. The protein sample was separated by SDS-PAGE, after mixing with 2×SDS buffer and heating at 100° C. for 7 minutes. The separated proteins were moved to polyvinylidene difluoride (PVDF) membrane, and then developed with ECL system by using anti-mouse secondary antibody and blocking solution which comprises anti-myc (9E10, sc-40), anti-HA (Santa Cruz Biotechnology, sc-7392) and anti-β-actin (sc-47778) in 1:1,000 (w/w). As a result, when immunoprecipitation was performed by using anti-myc (9E10, sc-40), poly-ubiquitin chain was formed by the binding of the ubiquitin to pcDNA3-myc-EPO WT, and thereby intense band indicating the presence of smear ubiquitin was produced (FIG. 46, lanes 3 and 4). Further, when the cells were treated with MG132 (proteasome inhibitor, 5 μg/ml) for 6 hrs, poly-ubiquitin chain formation was increased, and thus the more intense band indicating ubiquitin was appeared (FIG. 46, lane 4). Further, smaller amount of ubiquitin was detected for pcDNA3-myc-EPO mutant (K181R), since the mutant (K181R) was not bound to the ubiquitin (FIG. 47, lane 6). These results explain that insulin first binds to ubiquitin, and then is degraded through the polyubiquitin chain which is formed by ubiquitin-proteasome system.

3. Assessment of Erythropoietin Half-Life Using Protein Synthesis Inhibitor Cyclohexamide (CHX)

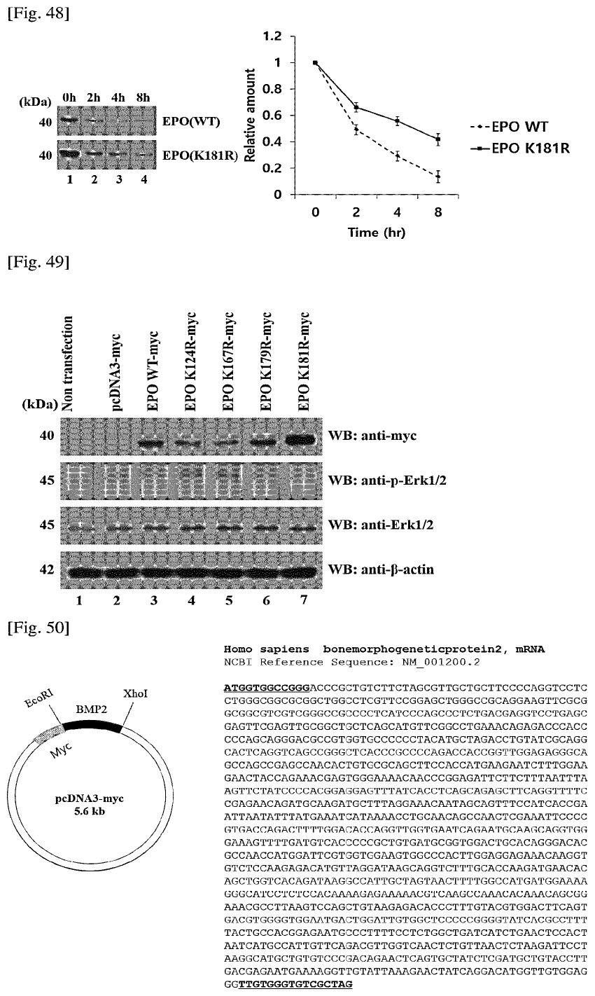

The HEK 293T cell was transfected with 2 μg of pcDNA3-myc-EPO WT, pcDNA3-myc-EPO mutant (K124R), pcDNA3-myc-EPO mutant (K167R), pcDNA3-myc-EPO mutant (K179R) and pcDNA3-myc-EPO mutant (K181R), respectively. 48 hrs after the transfection, the cells were treated with the protein synthesis inhibitor, cyclohexamide (CHX) (Sigma-Aldrich) (100 μg/ml), and then the half-life of each protein was detected at 2 hrs, 4 hrs and 8 hrs after the treatment of inhibitor. As a result, the degradation of human erythropoietin was observed (FIG. 48). The half-life of human erythropoietin (EPO) was less than 4 hrs, while the half-life of pcDNA3-myc-EPO mutant (K181R) was prolonged to 8 hrs or more, as shown in FIG. 48.

4. Signal Transduction by Erythropoietin (EPO) and the Substituted Erythropoietin (EPO) in Cells

It was reported that if the EPO is administered, it regulates cell cycle progression through Erk1/2 phosphorylation, and thus it has effects on hypoxia (J Hematol Oncol., 6, 65, 2013). In this experiment, we examined the signal transduction by erythropoietin (EPO) and erythropoietin (EPO) mutant in cells. First, the HepG2 cell (ATCC, AB-8065) was starved for 8 hrs, and then transfected by using 3 μg of pcDNA3-myc-EPO WT, pcDNA3-myc-EPO mutant (K124R), pcDNA3-myc-EPO mutant (K167R), pcDNA3-myc-EPO mutant (K179R) and pcDNA3-myc-EPO mutant (K181R), respectively. 2 days after the transfection, the proteins were extracted from the cells and quantified. Western blot was performed to analyze the signal transduction in the cells. The proteins separated from the HepG2 cell transfected with respective pcDNA3-myc-EPO WT, pcDNA3-myc-EPO mutant (K124R), pcDNA3-myc-EPO mutant (K167R), pcDNA3-myc-EPO mutant (K179R) and pcDNA3-myc-EPO mutant (K181R) were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-Erk1/2 (9B3, Abfrontier LF-MA0134), anti-phospho-Erk1/2 (Thr202/Tyr204, Abfrontier LF-PA0090) and anti-β-actin (sc-47778) in 1:1,000 (w/w). As a result, pcDNA3-myc-EPO mutant (K124R), pcDNA3-myc-EPO mutant (K167R), pcDNA3-myc-EPO mutant (K179R) and pcDNA3-myc-EPO mutant (K181R) showed the same or increased phospho-Erk1/2 signal transduction in HepG2 cell, in comparison to the pcDNA3-myc-EPO wild type (FIG. 49).

### Example 8: The Analysis of Ubiquitination and Half-Life Increase of Bone Morphogenetic Protein 2 (BMP2), and the Analysis of Signal Transduction in Cells

1. Bone Morphogenetic Protein 2 (BMP2) Expression Vector Cloning and Protein Expression

(1) Bone Morphogenetic Protein 2 (BMP2) Expression Vector Cloning

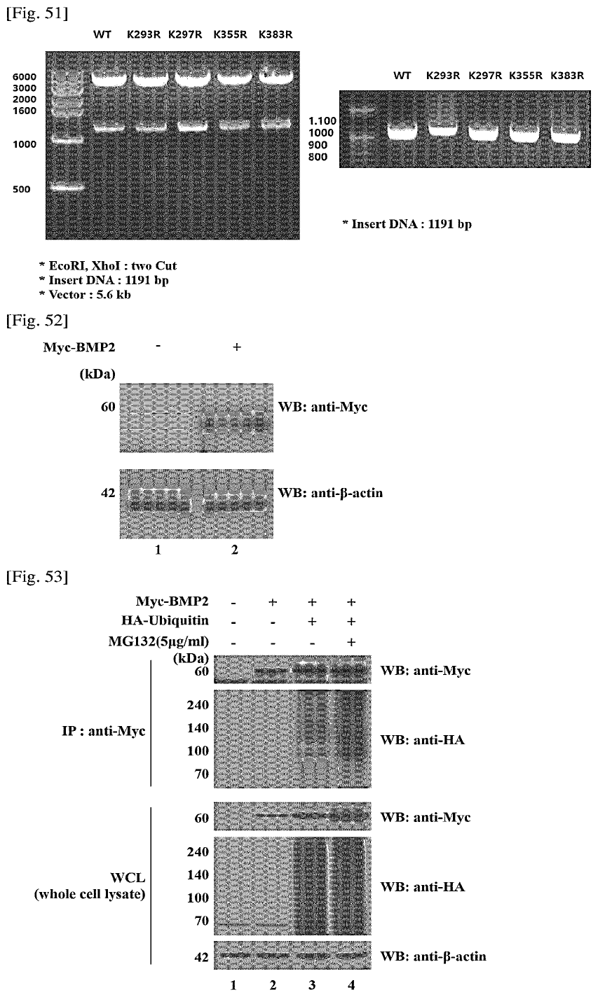

The bone morphogenetic protein 2 (BMP2) DNA amplified by PCR was treated with EcoRI and XhoI, and then ligated to pcDNA3-myc vector (5.6 kb) previously digested with the same enzyme (FIG. 50, BMP2 amino acid sequence: SEQ ID NO: 52). Then, agarose gel electrophoresis was carried out to confirm the presence of the DNA insert, after restriction enzyme digestion of the cloned vector (FIG. 51). The nucleotide sequences shown in underlined bold letters in FIG. 50 indicate the primer sets used for the PCR to confirm the cloned sites (FIG. 51). The PCR conditions are as follows, Step 1: at 94° C. for 3 minutes (1 cycle); Step 2: at 94° C. for 30 seconds; at 58° C. for 30 seconds; at 72° C. for 1 minute 30 seconds (25 cycles); and Step 3: at 72° C. for 10 minutes (1 cycle), and then held at 4° C. For the assessment of the expression of proteins encoded by cloned DNA, western blot was carried out with anti-myc antibody (9E10, sc-40) to myc of pcDNA3-myc vector shown in the map of FIG. 50. The western blot result showed that the BMP2 bound to myc was expressed well. The normalization with actin assured that proper amount of protein was loaded (FIG. 52).

(2) Lysine (Lysine, K) Residue Substitution

Lysine residue was replaced with arginine (Arginine, R) using site-directed mutagenesis. The following primer sets were used for PCR to prepare the substituted DNAs.

(BMP2 K293R)
FP
(SEQ ID NO: 53)
5′-GAAACGCCTTAGGTCCAGCTGTAAGAGAC-3′,
RP
(SEQ ID NO: 54)
5′-GTCTCTTACAGCTGGACCTAAGGCGTTTC 3′;
(BMP2 K297R)
FP
(SEQ ID NO: 55)
5′-TTAAGTCCAGCTGTAGGAGACACCCTTTGT-3′,
RP
(SEQ ID NO: 56)
5′-ACAAAGG GTGTCTCCTACAGCTGGACTTAA-3′;
(BMP2 K355R)
FP
(SEQ ID NO: 57)
5′-GTTAACTCTAGGATTCCTAAGGC-3′,
RP
(SEQ ID NO: 58)
5′-GC CTTAGGAATCCTAGAGTTAAC-3′;
and
(BMP2 K383R)
FP
(SEQ ID NO: 59)
5′-GGTTGTATTAAGGAACTATCAGGAC-3′,
RP
(SEQ ID NO: 60)
5′-GT CCTGATAGTTCCTTAATACAACC-3′.

Five plasmid DNAs each of which one or more which lysine residues were replaced with arginine (K→R) were prepared by using pcDNA3-myc-BMP2 as a template (Table 8).

TABLE 8

| Lysine(K) residue site | BMP2 construct, replacement of K with R |
|---|---|
| 293 | pcDNA3-myc-BMP2 (K293R) |
| 297 | pcDNA3-myc-BMP2 (K297R) |
| 355 | pcDNA3-myc-BMP2 (K355R) |
| 383 | pcDNA3-myc-BMP2 (K383R) |

2. In Vivo Ubiquitination Analysis

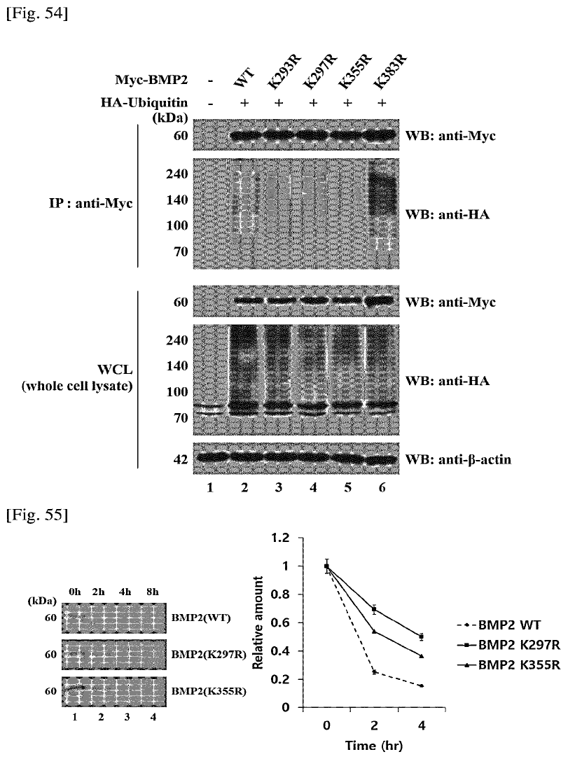

The HEK 293T cell was transfected with pcDNA3-myc-BMP2 WT and the plasmid encoding pMT123-HA-ubiquitin. For the analysis of the ubiquitination level, pcDNA3-myc-BMP2 WT 2 μg and pMT123-HA-ubiquitin DNA 1 μg were co-transfected into the cell. 24 hrs after the transfection, the cell was treated with MG132 (proteasome inhibitor, 5 μg/e) for 6 hrs, thereafter immunoprecipitation analysis was carried out (FIG. 53). Then, the HEK 293T cells were transfected with the plasmids encoding pcDNA3-myc-BMP2 WT, pcDNA3-myc-BMP2 mutant (K293R), pcDNA3-myc-BMP2 mutant (K297R), pcDNA3-myc-BMP2 mutant (K355R), pcDNA3-myc-BMP2 mutant (K383R) and pMT123-HA-ubiquitin, respectively. For the analysis of the ubiquitination level, the cell was co-transfected with 1 μg of pMT123-HA-ubiquitin DNA, and with respective 2 μg of pcDNA3-myc-BMP2 WT, pcDNA3-myc-BMP2 mutant (K62R), pcDNA3-myc-BMP2 mutant (K124R), pcDNA3-myc-BMP2 mutant (K153R) and pcDNA3-myc-BMP2 mutant (K158R). Next, 24 hrs after the transfection, immunoprecipitation was carried out (FIG. 54). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution. The protein sample was separated by SDS-PAGE, after mixing with 2×SDS buffer and heating at 100° C. for 7 minutes. The separated proteins were moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), poly-ubiquitin chain was formed by the binding of the ubiquitin to pcDNA3-myc-BMP2 WT, and thereby intense band indicating the presence of smear ubiquitin was detected (FIG. 53, lanes 3 and 4). Further, when the cell was treated with MG132 (proteasome inhibitor, 5 μg/ml) for 6 hrs, poly-ubiquitination formation was increased and thus the more intense band indicating ubiquitin was appeared (FIG. 53, lane 4). Further, as for the pcDNA3-myc-BMP2 mutant (K293R), pcDNA3-myc-BMP2 mutant (K297R) and pcDNA3-myc-BMP2 mutant (K355R), the band was less intense than the wild type, and smaller amount of ubiquitin was detected since pcDNA3-myc-BMP2 mutant (K293R), pcDNA3-myc-BMP2 mutant (K297R) and pcDNA3-myc-BMP2 mutant (K355R) were not bound to the ubiquitin (FIG. 54, lanes 3 to 5). These results represent that BMP2 first binds to ubiquitin, and then is degraded through the polyubiquitin chain which is formed by ubiquitin-proteasome system.

3. Assessment of BMP2 Half-Life Using Protein Synthesis Inhibitor Cyclohexamide (CHX)

The HEK 293T cell was transfected with 2 μg of pcDNA3-myc-BMP2 mutant (K293R), pcDNA3-myc-BMP2 mutant (K297R), pcDNA3-myc-BMP2 mutant (K355R) and pcDNA3-myc-BMP2 mutant (K383R), respectively. 48 hrs after the transfection, the cell was treated with the protein synthesis inhibitor, cyclohexamide (CHX) (Sigma-Aldrich) (100 μg/ml), and then the half-life of each protein was detected at 4 hrs and 8 hrs after the treatment of the inhibitor. As a result, the degradation of human BMP2 was observed (FIG. 55). The half-life of human BMP2 was less than 2 hrs, while the half-lives of human pcDNA3-myc-BMP2 mutant (K297R) and pcDNA3-myc-BMP2 mutant (K355R) were prolonged to 4 hrs or more, as shown in FIG. 55.

4. Signal Transduction by BMP2 and the Substituted BMP2 in Cells.

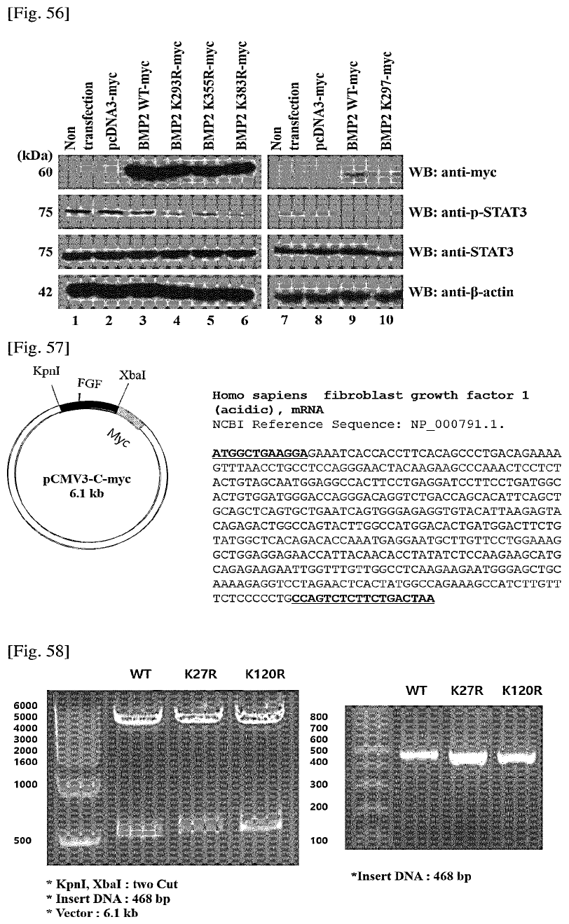

Bone morphogenetic protein-2 (BMP2) is known to inactivate STAT3 in various myeloma cells, and thereby induce apoptosis (Blood, 96, 2005-2011, 2000). In this experiment, we examined the signal transduction by BMP2 and the substituted BMP2 in cell. First, the HepG2 cell was starved for 8 hrs, and then transfected by using 3 μg of pcDNA3-myc-BMP2 WT, pcDNA3-myc-BMP2 mutant (K293R), pcDNA3-myc-BMP2 mutant (K297R), pcDNA3-myc-BMP2 mutant (K355R) and pcDNA3-myc-BMP2 mutant (K383R), respectively. 2 days after the transfection, the proteins were extracted from the cells and quantified. Western blot was performed to analyze the signal transduction in cells. The proteins separated from the HepG2 cell transfected with respective pcDNA3-myc-BMP2 WT, pcDNA3-myc-BMP2 mutant (K293R), pcDNA3-myc-BMP2 mutant (K297R), pcDNA3-myc-BMP2 mutant (K355R) and pcDNA3-myc-BMP2 mutant (K383R) were moved to PVDF membrane. Then, the proteins were developed with ECL system using anti-rabbit and anti-mouse secondary antibodies and blocking solution which comprises anti-STAT3 (sc-21876), anti-phospho-STAT3 (Y705, cell signaling 9131S) and anti-β-actin (sc-47778) in 1:1,000 (w/w). As a result, pcDNA3-myc-BMP2 mutant (K293R), pcDNA3-myc-BMP2 mutant (K297R), pcDNA3-myc-BMP2 mutant (K355R) and pcDNA3-myc-BMP2 mutant (K383R) showed the same or increased phospho-STAT3 signal transduction in HepG2 cell in comparison to the wild type (FIG. 56).

### Example 9: The Analysis of Ubiquitination and Half-Life Increase of Fibroblast Growth Factor-1 (FGF-1), and the Analysis of Signal Transduction in Cells

1. Fibroblast Growth Factor-1 (FGF-1) Expression Vector Cloning and Protein Expression

(1) Fibroblast Growth Factor-1 (FGF-1) Expression Vector Cloning

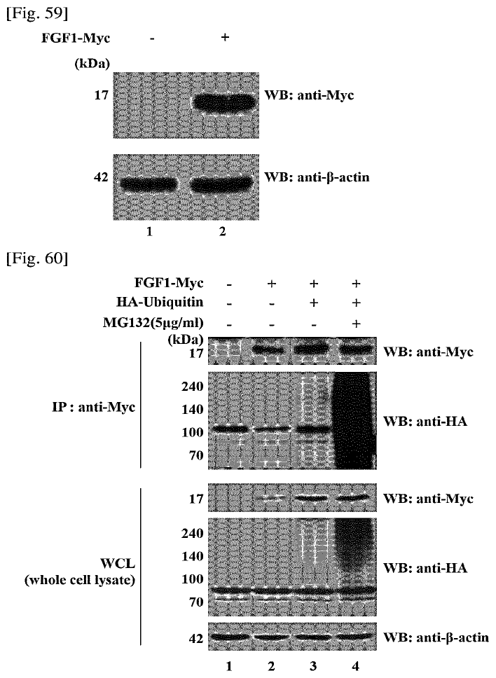

The fibroblast growth factor-1 (FGF-1) DNA amplified by PCR was treated with KpnI and XbaI, and then ligated to pCMV3-C-myc vector (6.1 kb) previously digested with the same enzyme (FIG. 57, FGF-1 amino acid sequence: SEQ ID NO: 61). Then, agarose gel electrophoresis was carried out to confirm the presence of the DNA insert, after restriction enzyme digestion of the cloned vector (FIG. 58). The nucleotide sequences shown in underlined bold letters in FIG. 57 indicate the primer sets used for the PCR to confirm the cloned sites (FIG. 58). The PCR conditions are as follows, Step 1: at 94° C. for 3 minutes (1 cycle); Step 2: at 94° C. for 30 seconds; at 58° C. for 30 seconds; at 72° C. for 30 seconds (25 cycles); and Step 3: at 72° C. for 10 minutes (1 cycle), and then held at 4° C. For the assessment of the expression of proteins encoded by cloned DNA, western blot was carried out with anti-myc antibody (9E10, sc-40) to myc of pcDNA3-myc vector shown in the map of FIG. 57. The western blot result showed that the FGF-1 bound to myc was expressed well. The normalization with actin assured that proper amount of protein was loaded (FIG. 59).

(2) Lysine (Lysine, K) Residue Substitution

Lysine residue was replaced with arginine (Arginine, R) using site-directed mutagenesis. The following primer sets were used for PCR to prepare the substituted plasmid DNAs.

(FGF-1 K27R)
FP
(SEQ ID NO: 62)
5′-AAGAAGCCCAGACTCCTCTAC-3′,
RP
(SEQ ID NO: 63)
5′-GTAGAGGAGTCTGGGCTTCTT-3′;
and
(FGF-1 K120R)
FP
(SEQ ID NO: 64)
5′-CATGCAGAGAGGAATTGGTTT-3′,
RP
(SEQ ID NO: 65)
5′-AAACCAATTCCTCTCTGCATG-3′.

Two plasmid DNAs each of which one or more lysine residues were replaced by arginine (K→R) were prepared by using pCMV3-C-myc-FGF-1 as a template (Table 9).

TABLE 9

| Lysine(K) residue site | FGF-1 construct, replacement of K with R |
|---|---|
| 27 | pCMV3-C-myc-FGF-1 (K27R) |
| 120 | pCMV3-C-myc-FGF-1 (K120R) |

2. In Vivo Ubiquitination Analysis

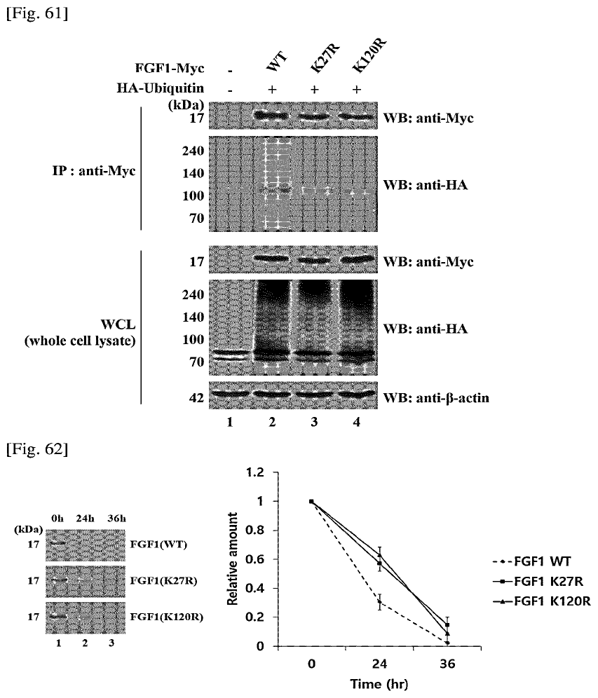

The HEK 293T cell was transfected with the plasmid encoding pCMV3-C-myc-FGF-1 WT and pMT123-HA-ubiquitin. For the analysis of the ubiquitination level, pCMV3-C-myc-FGF-1 WT 2 μg and pMT123-HA-ubiquitin DNA 1 μg were co-transfected into the cells. 24 hrs after the transfection, the cells were treated with MG132 (proteasome inhibitor, 5 μg/ml) for 6 hrs, thereafter immunoprecipitation analysis was carried out (FIG. 60). Then, the HEK 293T cells were transfected with the plasmids encoding pCMV3-C-myc-FGF-1 WT, pCMV3-C-myc-FGF-1 mutant (K27R), pCMV3-C-myc-FGF-1 mutant (K120R) and pMT123-HA-ubiquitin, respectively. For the analysis of the ubiquitination level, the cell was co-transfected with 1 μg of pMT123-HA-ubiquitin DNA, and respective with 2 μg of pCMV3-C-myc-FGF-1 WT, pCMV3-C-myc-FGF-1 mutant (K27R) and pCMV3-C-myc-FGF-1 (K120R). Next, 24 hrs after the transfection, immunoprecipitation was carried out (FIG. 61). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution.

The protein sample was separated by SDS-PAGE, after mixing with 2×SDS buffer and heating at 100° C., for 7 minutes. The separated proteins were moved to polyvinylidene difluoride (PVDF) membrane, and then developed with ECL system using anti-mouse secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), poly-ubiquitin chain was formed by the binding of the ubiquitin to pcDNA3-myc-FGF-1 WT, and thereby intense band indicating the presence of smear ubiquitin was detected (FIG. 60, lanes 3 and 4). Further, when the cells were treated with MG132 (proteasome inhibitor, 5 μg/ml) for 6 hrs, poly-ubiquitin chain formation was increased, and thus the more intense band indicating ubiquitin was appeared (FIG. 60, lane 4). Further, as for the pCMV3-C-myc-FGF-1 mutant (K27R) and pCMV3-C-myc-FGF-1 mutant (K120R), the band was less intense than the wild type, and smaller amount of ubiquitin was detected since pCMV3-C-myc-FGF-1 mutant (K27R) and pCMV3-C-FGF-1 mutant (K120R) were not bound to the ubiquitin (FIG. 61, lanes 3 and 4). These results represent that FGF-1 first binds to ubiquitin, and then is degraded through the polyubiquitin chain which is formed by ubiquitin-proteasome system.

3. Assessment of FGF-1 Half-Life Using Protein Synthesis Inhibitor Cyclohexamide (CHX)

The HEK 293T cell was transfected with 2 μg of pCMV3-C-myc-FGF-1 WT, pCMV3-C-myc-FGF-1 mutant (K27R) and pCMV3-C-myc-FGF-1 mutant (K120R), respectively. 48 hrs after the transfection, the cells were treated with the protein synthesis inhibitor, cyclohexamide (CHX) (Sigma-Aldrich) (100 μg/ml), and then the half-life of each protein was detected for 24 hrs and 36 hrs after the treatment of the inhibitor. As a result, the degradation of human FGF-1 was observed (FIG. 62). The half-life of human FGF-1 was less than 1 day, while the half-lives of human pCMV3-C-myc-FGF-1 mutant (K27R) and pCMV3-C-myc-FGF-1 mutant (K120R) were prolonged to 1 day or more, as shown in FIG. 62.

4. Signal Transduction by FGF-1 and the Substituted FGF-1 in Cells

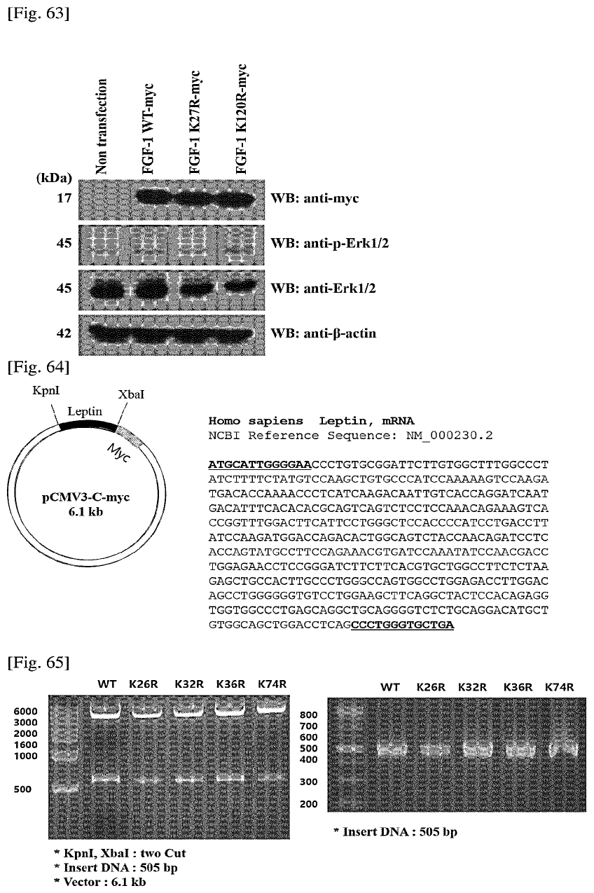

It was reported that when the HEK293 cell is treated with the recombinant FGF-1, Erk 1/2 phosphorylation increases (Nature, 513(7518), 436-439, 2014). In this experiment, we examined the signal transduction by FGF-1 and the substituted FGF-1 in cells. First, the HepG2 cell (ATCC, AB-8065) was starved for 8 hrs, and then transfected by using 3 ag of pCMV3-C-myc-FGF-1 WT, pCMV3-C-myc-FGF-1 mutant (K27R) and pCMV3-C-myc-FGF-1 mutant (K120R), respectively. 2 days after the transfection, the protein was extracted from the cells and quantified. Western blot was performed to analyze the signal transduction in the cells. The proteins separated from the HepG2 cell transfected with respective pCMV3-C-myc-FGF-1 WT, pCMV3-C-myc-FGF-1 mutant (K27R) and pCMV3-C-myc-FGF-1 mutant (K120R) were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-Erk1/2 (9B3, Abfrontier LF-MA0134), anti-phospho-Erk1/2 (Thr202/Tyr204, Abfrontier LF-PA0090) and anti-β-actin (sc-47778) in 1:1,000 (w/w). As a result, pCMV3-C-myc-FGF-1 mutant (K27R) and pCMV3-C-myc-FGF-1 mutant (K120R) showed the same or increased phospho-ERK1/2 signal transduction in HepG2 cell in comparison to the wild type (FIG. 63).

### Example 10: The Analysis of Ubiquitination and Half-Life Increase of Leptin, and the Analysis of Signal Transduction in Cells

1. Leptin Expression Vector Cloning and Protein Expression

(1) Leptin Expression Vector Cloning

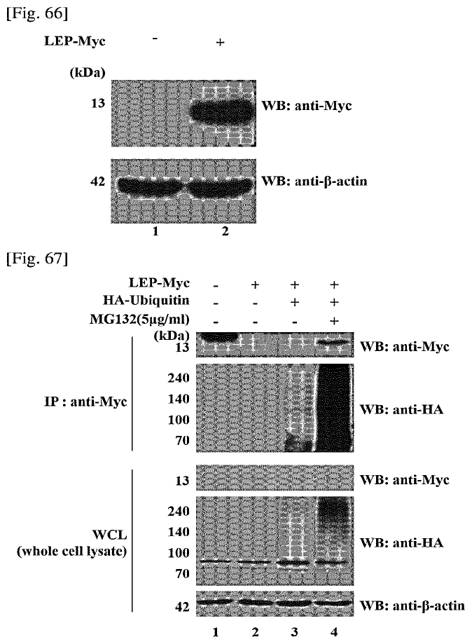

The Leptin DNA amplified by PCR was treated with KpnI and XbaI, and then ligated to pCMV3-C-myc vector (6.1 kb) previously digested with the same enzyme (FIG. 64, Leptin amino acid sequence: SEQ ID NO: 66). Then, agarose gel electrophoresis was carried out to confirm the presence of the DNA insert, after restriction enzyme digestion of the cloned vector (FIG. 65). The nucleotide sequences shown in underlined bold letters in FIG. 64 indicate the primer sets used for the PCR to confirm the cloned sites (FIG. 65). The PCR conditions are as follows, Step 1: at 94° C. for 3 minutes (1 cycle); Step 2: at 94° C. for 30 seconds; at 58° C. for 30 seconds; at 72° C. for 45 seconds (25 cycles); and Step 3: at 72° C. for 10 minutes (1 cycle), and then held at 4° C. For the assessment of the expression of proteins encoded by cloned DNA, western blot was carried out with anti-myc antibody (9E10, sc-40) to myc of pCMV3-C-myc vector shown in the map of FIG. 64. The western blot results showed that the Leptin protein bound to myc was expressed well. The normalization with actin assured that proper amount of protein was loaded (FIG. 66).

(2) Lysine (Lysine, K) Residue Substitution

Lysine residue was replaced with arginine (Arginine, R) by using site-directed mutagenesis. The following primer sets were used for PCR to prepare the substituted plasmid DNAs.

(Leptin K26R)
FP
(SEQ ID NO: 67)
5′-CCCATCCAAAAGGTCCAAGAT-3′,
RP
(SEQ ID NO: 68)
5′-ATCTTGGACCTTTTGGATGGG-3′;
(Leptin K32R)
FP
(SEQ ID NO: 69)
5′-GATGACACCAAGACCCTCATC-3′,
RP
(SEQ ID NO: 70)
5′-GATGAGGGTCTTGGTGTCATC-3′;
(Leptin K36R)
FP
(SEQ ID NO: 71)
5′-ACCCTCATCAGGACAATTGTC-3′,
RP
(SEQ ID NO: 72)
5′-GACAATTGTCCTGATGAGGGT-3′;
and
(Leptin K74R)
FP
(SEQ ID NO: 73)
5′-ACCTTATCCAGGATGGACCAG-3′,
RP
(SEQ ID NO: 74)
5′-CTGGTCCATCCTGGATAAGGT-3′.

Four plasmid DNAs each of which one or more lysine residues were replaced by arginine (K→R) were produced by using pCMV3-C-myc-Leptin as a template (Table 10).

TABLE 10

| Lysine(K) residue site | Leptin construct, replacement of K with R |
|---|---|
| 26 | pCMV3-C-myc-Leptin (K26R) |
| 32 | pCMV3-C-myc-Leptin (K32R) |
| 36 | pCMV3-C-myc-Leptin (K36R) |
| 74 | pCMV3-C-myc-Leptin (K74R) |

2. In Vivo Ubiquitination Analysis

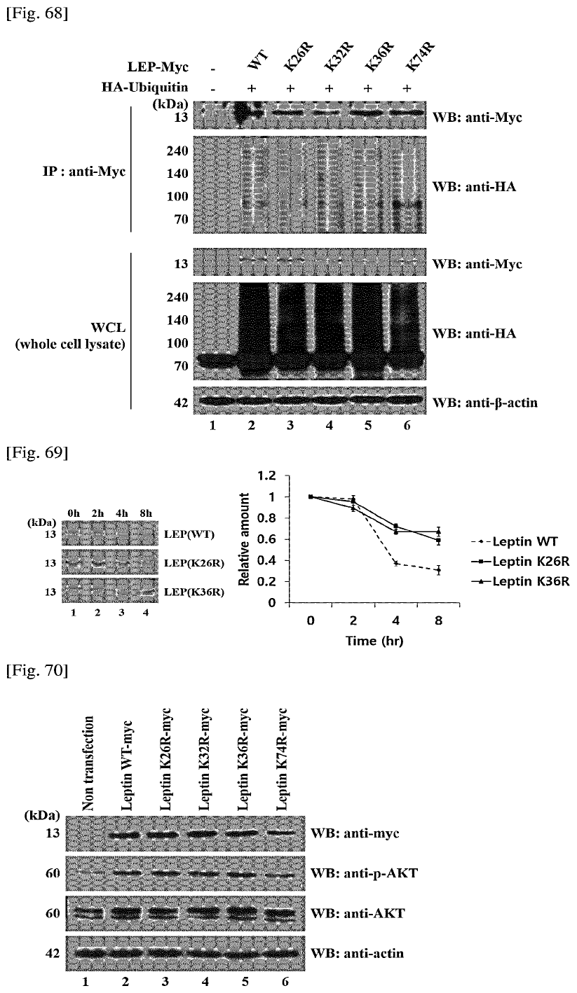

The HEK 293T cell was transfected with the plasmid encoding pCMV3-C-myc-Leptin WT and pMT123-HA-ubiquitin. For the analysis of the ubiquitination level, pCMV3-C-myc-Leptin WT 6 μg and pMT123-HA-ubiquitin DNA 1 μg were co-transfected into the cells. 24 hrs after the transfection, the cells were treated with MG132 (proteasome inhibitor, 5 μg/ml) for 6 hrs, thereafter immunoprecipitation analysis was carried out (FIG. 67). Then, the HEK 293T cells were transfected with the plasmids encoding pCMV3-C-myc-Leptin WT, pCMV3-C-myc-Leptin mutant (K26R), pCMV3-C-myc-Leptin mutant (K32R), pCMV3-C-myc-Leptin mutant (K36R), pCMV3-C-myc-Leptin mutant (K74R) and pMT123-HA-ubiquitin, respectively. For the analysis of the ubiquitination level, the cells were co-transfected with 1 μg of pMT123-HA-ubiquitin DNA, and with respective 6 μg of pCMV3-C-myc-Leptin WT, pCMV3-C-myc-Leptin mutant (K26R), pCMV3-C-myc-Leptin mutant (K32R), pCMV3-C-myc-Leptin mutant (K36R) and pCMV3-C-myc-Leptin mutant (K74R). Next, 24 hrs after the transfection, immunoprecipitation was carried out (FIG. 68). The protein sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution. The protein sample was separated by SDS-PAGE, after mixing with 2×SDS buffer and heating at 100° C., for 7 minutes. The separated proteins were moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), polyubiquitin chain was formed by the binding of the ubiquitin to pCMV3-C-myc-Leptin-1 WT, and thereby intense band indicating the presence of smear ubiquitin was detected (FIG. 67, lanes 3 and 4). Further, when the cells were treated with MG132 (proteasome inhibitor, 5 μg/ml) for 6 hrs, poly-ubiquitin chain formation was increased, and thus the more intense band indicating ubiquitin was produced (FIG. 67, lane 4). Further, as for the pCMV3-C-myc-Leptin mutant (K26R), pCMV3-C-myc-Leptin mutant (K36R) and pCMV3-C-myc-Leptin mutant (K74R), the band was less intense than the wild type, and smaller amount of ubiquitin was detected since the mutants were not bound to the ubiquitin (FIG. 68, lanes 3, 5 and 6). These results show that insulin first binds to ubiquitin, and then is degraded through the polyubiquitin chain which is formed by ubiquitin-proteasome system.

3. Assessment of Leptin Half-Life Using Protein Synthesis Inhibitor Cyclohexamide (CHX)

The HEK 293T cell was transfected with 6 μg of pCMV3-C-myc-Leptin WT, pCMV3-C-myc-Leptin mutant (K26R), pCMV3-C-myc-Leptin mutant (K32R), pCMV3-C-myc-Leptin mutant (K36R) and pCMV3-C-myc-Leptin mutant (K74R), respectively. 48 hrs after the transfection, the cells were treated with the protein synthesis inhibitor, cyclohexamide (CHX) (Sigma-Aldrich) (100 μg/ml), and then the half-life of each protein was detected at 2, 4 and 8 hrs after the treatment of the inhibitor. As a result, the degradation of human Leptin was observed (FIG. 69). The half-life of human Leptin was about 4 hr, while the half-lives of human pCMV3-C-myc-Leptin mutant (K26R) and pCMV3-C-myc-Leptin mutant (K36R) were prolonged to 8 hrs or more, as shown in FIG. 69.

4. Signal Transduction by Leptin and the Substituted Leptin in Cells

It was reported that the Leptin enhances AKT phosphorylation in breast cancer cells (Cancer Biol Ther., 16(8), 1220-1230, 2015), and reported that stimulates the growth of cancer cells through PI3K/AKT signal transduction uterine cancer (Int J Oncol., 49(2), 847, 2016). In this experiment, we examined the signal transduction by Leptin and the substituted Leptin in a cell. First, the HepG2 cell was starved for 8 hrs, and then transfected by using 6 μg of pCMV3-C-myc-Leptin WT, pCMV3-C-myc-Leptin mutant (K26R), pCMV3-C-myc-Leptin mutant (K32R), pCMV3-C-myc-Leptin mutant (K36R) and pCMV3-C-myc-Leptin mutant (K74R), respectively. 2 days after the transfection, the proteins were extracted from the cells and quantified. Western blot was performed to analyze the signal transduction in the cells. The proteins separated from the HepG2 cells transfected with respective pCMV3-C-myc-Leptin WT, pCMV3-C-myc-Leptin mutant (K26R), pCMV3-C-myc-Leptin mutant (K32R), pCMV3-C-myc-Leptin mutant (K36R) and pCMV3-C-myc-Leptin mutant (K74R), were moved to PVDF membrane. Then, the proteins were developed with ECL system using anti-rabbit and anti-mouse secondary antibodies and blocking solution which comprises anti-myc (9E10, sc-40), anti-AKT (H-136, sc-8312), anti-phospho-AKT (S473, Cell Signaling 92715) and anti-β-actin (sc-47778) in 1:1,000 (w/w). As a result, pCMV3-C-myc-Leptin mutant (K26R), pCMV3-C-myc-Leptin mutant (K32R), pCMV3-C-myc-Leptin mutant (K36R) and pCMV3-C-myc-Leptin mutant (K74R) showed significantly increased phospho-AKT signal transduction in HepG2 cell, in comparison to the controls (FIG. 70).

### Example 11: The Analysis of Ubiquitination and Half-Life Increase of Vascular Endothelial Growth Factor A (VEGFA), and the Analysis of Signal Transduction in Cells

1. Vascular Endothelial Growth Factor A (VEGFA) Expression Vector Cloning and Protein Expression

(1) Vascular Endothelial Growth Factor A (VEGFA) Expression Vector Cloning

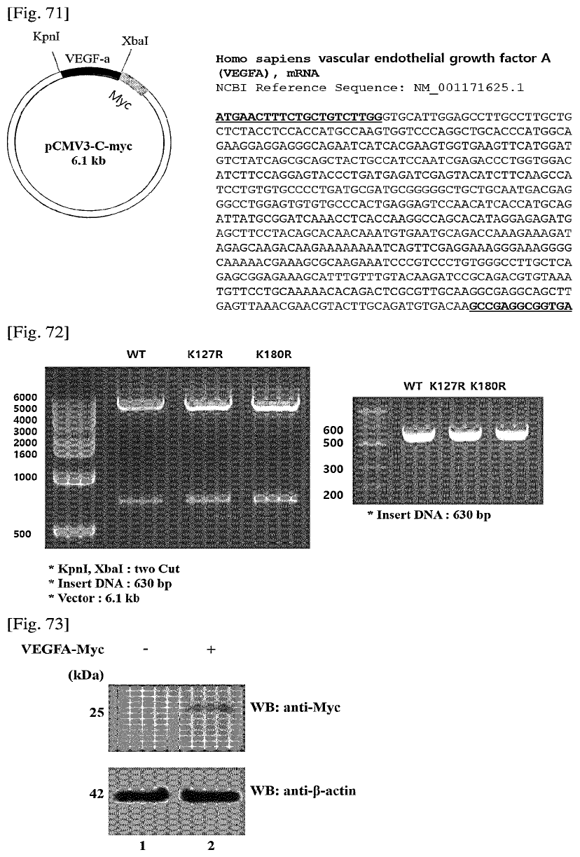

The vascular endothelial growth factor A (VEGFA) DNA amplified by PCR was treated with KpnI and XbaI, and then ligated to pCMV3-C-myc vector (6.1 kb) previously digested with the same enzyme (FIG. 71, VEGFA amino acid sequence: SEQ ID NO: 75). Then, agarose gel electrophoresis was carried out to confirm the presence of the DNA insert, after restriction enzyme digestion of the cloned vector (FIG. 72). The nucleotide sequences shown in underlined bold letters in FIG. 71 indicate the primer sets used for the PCR to confirm the cloned sites (FIG. 72). The PCR conditions are as follows, Step 1: at 94° C. for 3 minutes (1 cycle); Step 2: at 94° C. for 30 seconds; at 58° C. for 30 seconds; at 72° C. for 1 minute (25 cycles); and Step 3: at 72° C. for 10 minutes (1 cycle), and then held at 4° C. For the assessment of the expression of proteins encoded by cloned DNA, western blot was carried out with anti-myc antibody (9E10, sc-40) to myc of pCMV3-C-myc vector shown in the map of FIG. 71. The western blot result showed that the VEGFA bound to myc was expressed well. The normalization with actin assured that proper amount of protein was loaded (FIG. 73).

(2) Lysine (Lysine, K) Residue Substitution

Lysine residue was replaced with arginine (Arginine, R) using site-directed mutagenesis. The following primer sets were used for PCR to prepare the substituted plasmid DNAs.

(VEGFA K127R)
FP
(SEQ ID NO: 76)
5′-TACAGCACAACAGATGTGAATGCAGACC-3′,
RP
(SEQ ID NO: 77)
5′-GGTCTGCATTCACATCTGTTGTGCTGTA-3′;
and
(VEGFA K180R)
FP
(SEQ ID NO: 78)
5′-ATCCGCAGACGTGTAGATGTTCCTGCA-3′,
RP
(SEQ ID NO: 79)
5′-TGCAGGAACATCT ACACGTCTGCGGAT-3′.

Two plasmid DNAs each of which one or more lysine residues were replaced with arginine (K→R) were prepared by using pCMV3-C-myc-VEGFA DNA as a template (Table 11).

TABLE 11

| Lysine(K) residue site | VEGFA construct, replacement of K with R |
|---|---|
| 127 | pCMV3-C-myc-VEGFA (K127R) |
| 180 | pCMV3-C-myc-VEGFA (K180R) |

2. In Vivo Ubiquitination Analysis

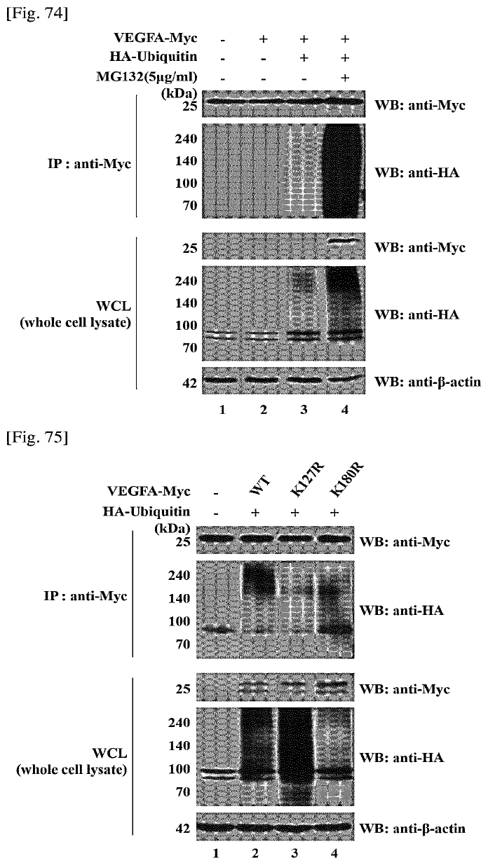

The HEK 293T cell was transfected with the plasmid encoding pCMV3-C-myc-VEGFA WT and pMT123-HA-ubiquitin. For the analysis of the ubiquitination level, pCMV3-C-myc-VEGFA WT 6 μg and pMT123-HA-ubiquitin DNA 1 μg were co-transfected into the cells. 24 hrs after the transfection, the cells were treated with MG132 (proteasome inhibitor, 5 μg/ml) for 6 hrs, thereafter immunoprecipitation analysis was carried out (FIG. 74). Then, the HEK 293T cells were transfected with the plasmids encoding pCMV3-C-myc-VEGFA WT, pCMV3-C-myc-VEGFA mutant (K127R), pCMV3-C-myc-VEGFA mutant (K180R) and pMT123-HA-ubiquitin, respectively. For the analysis of the ubiquitination level, the cells were co-transfected with 1 μg of pMT123-HA-ubiquitin DNA, and respective with 6 μg of pCMV3-C-myc-VEGFA WT, pCMV3-C-myc-VEGFA mutant (K127R) and pCMV3-C-myc-VEGFA mutant (K180R). Next, 24 hrs after the transfection, the immunoprecipitation was carried out (FIG. 75). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution. The protein sample was separated by SDS-PAGE, after mixing with 2×SDS buffer and heating at 100° C., for 7 minutes.

The separated proteins were moved to polyvinylidene difluoride (PVDF) membrane, and then developed with ECL system using anti-mouse secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), poly-ubiquitin chain was formed by the binding of the ubiquitin to pCMV3-C-myc-VEGFA WT, and thereby intense band indicating the presence of smear ubiquitin was detected (FIG. 74, lanes 3 and 4). Further, when the cells were treated with MG132 (proteasome inhibitor, 5 μg/ml) for 6 hrs, poly-ubiquitin chain formation was increased and thus the more intense band indicating ubiquitin was appeared (FIG. 74, lane 4). Further, as for the pCMV3-C-myc-VEGFA mutant (K127R) and pCMV3-C-myc-VEGFA mutant (K180R), the band was less intense than the wild type, and smaller amount of ubiquitin was detected since the mutants were not bound to the ubiquitin (FIG. 75, lanes 3 and 4). These results represent that VEGFA first binds to ubiquitin, and then is degraded through the polyubiquitin chain which is formed by ubiquitin-proteasome system.

3. Assessment of VEGFA Half-Life Using Protein Synthesis Inhibitor Cyclohexamide (CHX)

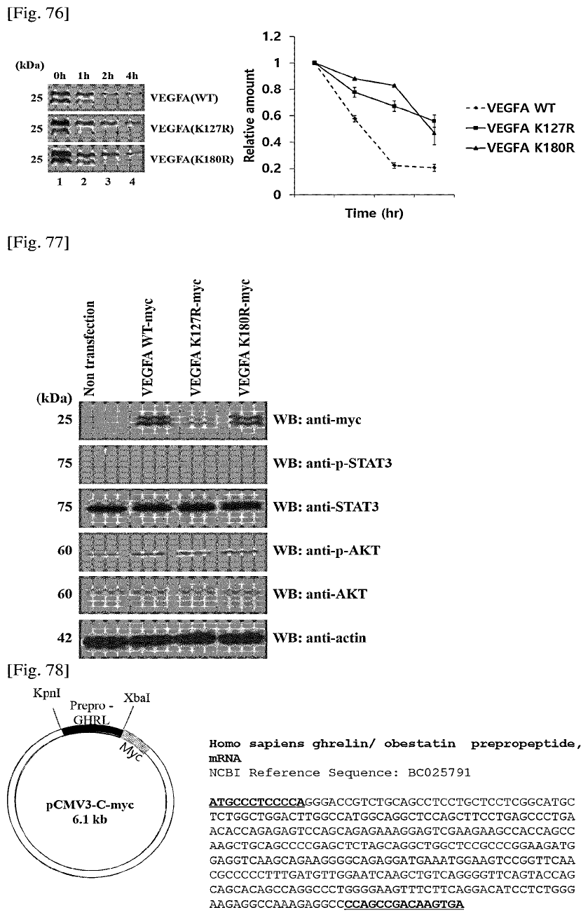

The HEK 293T cell was transfected with 6 μg of pCMV3-C-myc-VEGFA WT, pCMV3-C-myc-VEGFA mutant (K127R) and pCMV3-C-myc-VEGFA mutant (K180R), respectively. 48 hrs after the transfection, the cells were treated with the protein synthesis inhibitor, cyclohexamide (CHX) (Sigma-Aldrich) (100 μg/ml), and then the half-life of each protein was detected at 2, 4 and 8 hrs after the treatment of the inhibitor. As a result, the degradation of human VEGFA was observed (FIG. 76). The half-life of human VEGFA was less than 2 hrs, while the half-lives of human pCMV3-C-myc-VEGFA mutant (K127R) and pCMV3-C-myc-VEGFA mutant (K180R) was prolonged to 4 hrs or more, as shown in FIG. 76.

4. Examination of Signal Transduction by VEGFA and the Substituted VEGFA in Cells

The VEGFA relates to growth and proliferation of endothelial cells and functions in angiogenesis in cancer cells, while involves in PI3K/Akt/HIF-la pathway (Carcinogenesis, 34, 426-435, 2013). Further, the VEGF induces AKT phosphorylation (Kidney Int., 68, 1648-1659, 2005). In this experiment, we examined the signal transduction by VEGFA and the substituted VEGFA in cells. First, the HepG2 cell (ATCC, AB-8065) was starved for 8 hrs, and then transfected by using 6 μg of pCMV3-C-myc-VEGFA WT, pCMV3-C-myc-VEGFA mutant (K127R) and pCMV3-C-myc-VEGFA mutant (K180R), respectively. 2 days after the transfection, the proteins were extracted from the cells and quantified. Western blot was performed to analyze the signal transduction in the cells. The proteins separated from the HepG2 cell transfected with respective pCMV3-C-myc-VEGFA WT, pCMV3-C-myc-VEGFA mutant (K127R) and pCMV3-C-myc-VEGFA mutant (K180R) were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-myc (9E10, Santa Cruz Biotechnology, sc-40), snti-STAT3 (sc-21876), anti-phospho-STAT3 (Y705, cell signaling 9131S), anti-AKT (H-136, sc-8312), anti-phospho-AKT (S473, cell signaling 92715) and anti-β-actin (sc-47778) in 1:1,000 (w/w). As a result, pCMV3-C-myc-VEGFA mutant (K127R) and pCMV3-C-myc-VEGFA mutant (K180R) showed the same or increased phospho-STAT3 and phospho-AKT signal transduction in HepG2 cell in comparison to the wild type (FIG. 77).

### Example 12: The Analysis of Ubiquitination and Half-Life Increase of Appetite Stimulating Hormone Precursor (Ghrelin/Obestatin Preprohormone; Prepro-GHRL), and the Analysis of Signal Transduction in Cells

1. Prepro-GHRL Expression Vector Cloning and Protein Expression

(1) Prepro-GHRL Expression Vector Cloning

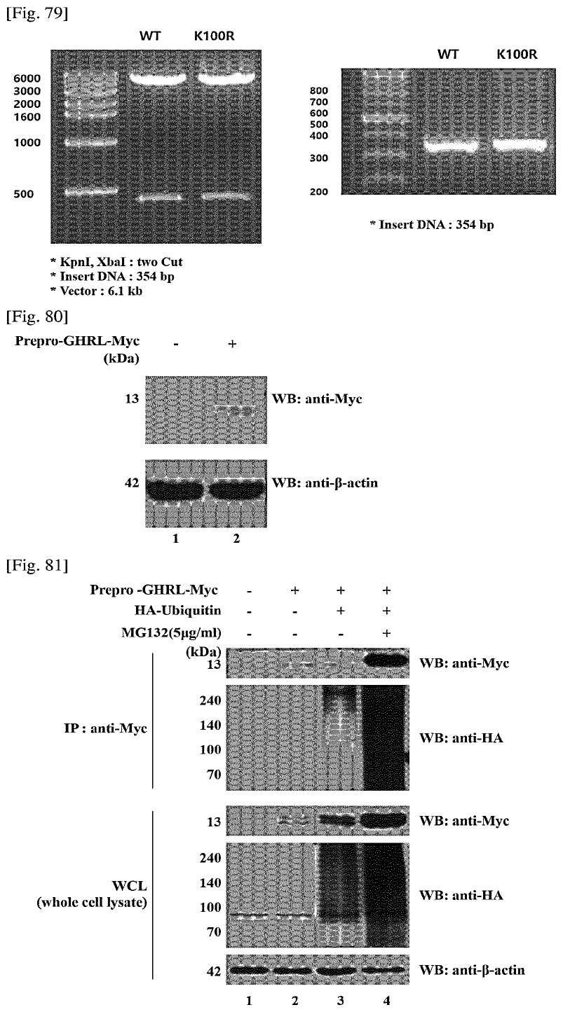

The prepro-GHRL DNA amplified by PCR was treated with KpnI and XbaI, and then ligated to pCMV3-C-myc vector (6.1 kb) previously digested with the same enzyme (FIG. 78, prepro-GHRL amino acid sequence: SEQ ID NO: 80). Then, agarose gel electrophoresis was carried out to confirm the presence of the DNA insert, after restriction enzyme digestion of the cloned vector (FIG. 79). The nucleotide sequences shown in underlined bold letters in FIG. 78 indicate the primer sets used for the PCR to confirm the cloned sites (FIG. 79). The PCR conditions are as follows, Step 1: at 94° C. for 3 minutes (1 cycle); Step 2: at 94° C. for 30 seconds; at 58° C. for 30 seconds; at 72° C. for 30 seconds (25 cycles); and Step 3: at 72° C. for 10 minutes (1 cycle), and then held at 4° C. For the assessment of the expression of proteins encoded by cloned DNA, western blot was carried out with anti-myc antibody (9E10, sc-40) to myc of pCMV3-C-myc vector shown in the map of FIG. 78. The western blot result showed that the appetite stimulating hormone precursor protein bound to myc was expressed well. The normalization with actin assured that proper amount of protein was loaded (FIG. 80).

(2) Lysine (Lysine, K) Residue Substitution

Lysine residue was replaced with arginine (Arginine, R) using site-directed mutagenesis. The following primer sets were used for PCR to prepare the substituted plasmid DNAs.

(prepro-GHRL K100R)
FP
(SEQ ID NO: 81)
5′-GCCCTGGGGAGGTTTCTTCAG-3′,
RP
(SEQ ID NO: 82)
5′-CTGAAGAAACCTCCCCAGGGC-3′.

A plasmid DNA of which lysine residue was replaced by arginine (K→R) was prepared using pCMV3-C-myc-prepro-GHRL as a template (Table 12).

TABLE 12

| Lysine(K) residue site | prepro-GHRL construct, replacement of K with R |
|---|---|
| 100 | pCMV3-C-myc-prepro-GHRL (K100R) |

2. In Vivo Ubiquitination Analysis

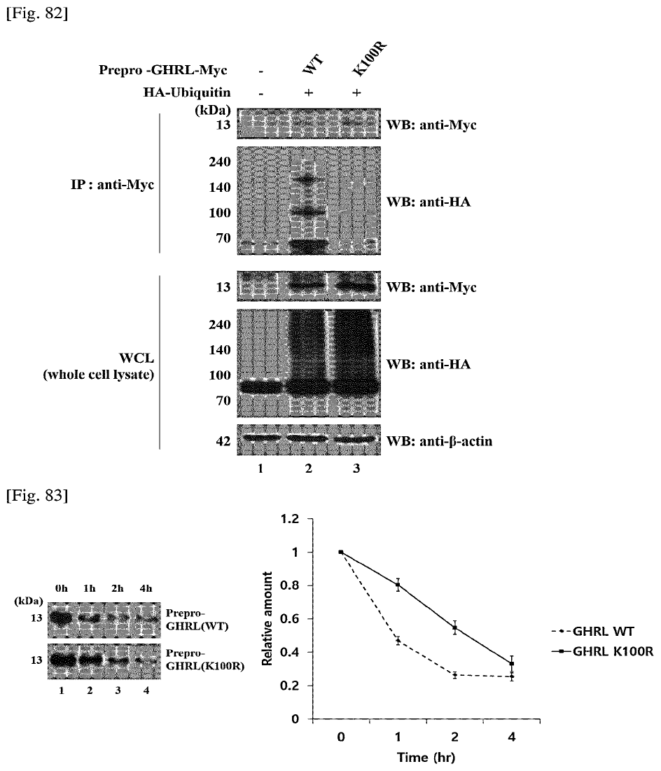

The HEK 293T cell was transfected with the plasmid encoding pCMV3-C-myc-prepro-GHRL WT and pMT123-HA-ubiquitin. For the analysis of the ubiquitination level, pCMV3-C-myc-prepro-GHRL WT 6 μg and pMT123-HA-ubiquitin DNA 1 μg were co-transfected into the cell. 24 hrs after the transfection, the cell was treated with MG132 (proteasome inhibitor, 5 μg/ml) for 6 hrs, thereafter immunoprecipitation analysis was carried out (FIG. 81). Then, the HEK 293T cells were transfected with the plasmids encoding pCMV3-C-myc-prepro-GHRL WT, pCMV3-C-myc-prepro-GHRL mutant (K100R) and pMT123-HA-ubiquitin, respectively. For the analysis of the ubiquitination level, the cells were co-transfected with 1 μg of pMT123-HA-ubiquitin DNA, and respective with 6 kg of pCMV3-C-myc-prepro-GHRL WT and pCMV3-C-myc-prepro-GHRL mutant (K100R). Next, 24 hrs after the transfection, immunoprecipitation was carried out (FIG. 82). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution. The protein sample was separated by SDS-PAGE, after mixing with 2×SDS buffer and heating at 100° C., for 7 minutes. The separated proteins were moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), poly-ubiquitin chain was formed by the binding of the ubiquitin to pCMV3-C-myc-prepro-GHRL WT, and thereby intense band indicating the presence of smear ubiquitin was detected (FIG. 81, lanes 3 and 4). Further, when the cell was treated with MG132 (proteasome inhibitor, 5 μg/ml) for 6 hrs, polyubiquitin chain formation was increased and thus the more intense band indicating ubiquitin was appeared (FIG. 81, lane 4). Further, as for the pCMV3-C-myc-prepro-GHRL mutant (K100R), the band was less intense than the wild type, and smaller amount of ubiquitin was detected since pCMV3-C-myc-prepro-GHRL mutant (K100R) was not bound to the ubiquitin (FIG. 82, lane 3). These results represent that prepro-GHRL first binds to ubiquitin, and then is degraded through the polyubiquitin chain which is formed by ubiquitin-proteasome system.

3. Assessment of Prepro-GHRL Half-Life Using Protein Synthesis Inhibitor Cyclohexamide (CHX)

The HEK 293T cell was transfected with 2 μg of pCMV3-C-myc-prepro-GHRL WT and pCMV3-C-myc-prepro-GHRL mutant (K100R), respectively. 48 hrs after the transfection, the cells were treated with the protein synthesis inhibitor, cyclohexamide (CHX) (Sigma-Aldrich) (100 μg/ink), and then the half-life of each protein was detected for 2, 4, and 8 hrs after the treatment of the inhibitor. As a result, the degradation of human prepro-GHRL was observed (FIG. 83). The half-life of human prepro-GHRL was less than 2 hr, while the half-life of the pCMV3-C-myc-prepro-GHRL mutant (K100R) was prolonged to 2 hr or more, as shown in FIG. 83.

4. Signal Transduction by Prepro-GHRL and the Substituted Prepro-GHRL in Cells

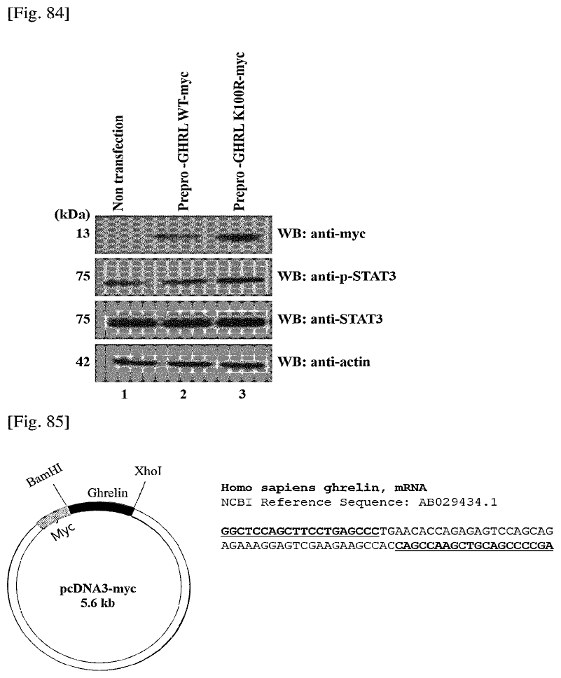

It was reported that the appetite stimulating hormone precursor regulates cell growth through the growth hormone secretagogue receptor (GHS-R), and enhances STAT3 via calcium regulation in vivo (Mol Cell Endocrinol., 285, 19-25, 2008). In this experiment, we examined the signal transduction by prepro-GHRL and the substituted prepro-GHRL in cells. First, the HepG2 cell was starved for 8 hrs, and then transfected by using 6 ag of pCMV3-C-myc-prepro-GHRL WT and pCMV3-C-myc-prepro-GHRL mutant (K100R), respectively. 2 days after the transfection, the proteins were extracted from the cells and quantified. Western blot was performed to analyze the signal transduction in cells. The proteins separated from the HepG2 cell (ATCC, AB-8065) transfected with respective pCMV3-C-myc-prepro-GHRL WT and pCMV3-C-myc-prepro-GHRL mutant (K100R) were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-myc (9E10, Santa Cruz Biotechnology, sc-40), anti-STAT3 (sc-21876), antiphospho-STAT3 (Y705, cell signaling 9131S) and anti-β-actin (sc-47778) in 1:1,000 (w/w). As a result, pCMV3-C-myc-prepro-GHRL mutant (K100R) showed the same or increased phospho-STAT3 signal transduction in HepG2 cells, in comparison to the wild type (FIG. 84).

### Example 13: The Analysis of Ubiquitination and Half-Life Increase of Ghrelin, and the Analysis of Signal Transduction in Cells

1. Ghrelin Expression Vector Cloning and Protein Expression

(1) Ghrelin Expression Vector Cloning

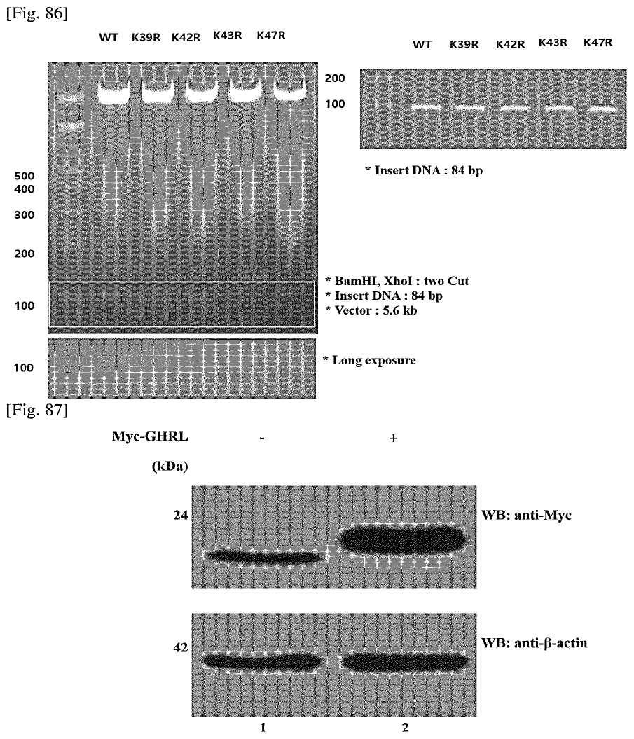

The appetite stimulating hormone (Ghrelin) DNA amplified by PCR was treated with BamHI and XhoII, and then ligated to pcDNA3-myc vector (5.6 kb) previously digested with the same enzyme (FIG. 85, Ghrelin amino acid sequence: SEQ ID NO: 83). Then, agarose gel electrophoresis was carried out to confirm the presence of the DNA insert, after restriction enzyme digestion of the cloned vector (FIG. 86). The nucleotide sequences shown in underlined bold letters in FIG. 85 indicate the primer sets used for the PCR to confirm the cloned sites (FIG. 86). The PCR conditions are as follows, Step 1: at 94° C. for 3 minutes (1 cycle); Step 2: at 94° C. for 30 seconds; at 58° C. for 30 seconds; at 72° C. for 20 seconds (25 cycles); and Step 3: at 72° C. for 10 minutes (1 cycle), and then held at 4° C. For the assessment of the expression of proteins encoded by cloned DNA, western blot was carried out with anti-myc antibody (9E10, sc-40) to myc of pcDNA3-myc vector shown in the map of FIG. 85. The western blot result showed that the appetite stimulating hormone (Ghrelin) pcDNA3-myc bound to myc was expressed well. The normalization with actin assured that proper amount of protein was loaded (FIG. 87).

(2) Lysine (Lysine, K) Residue Substitution

Lysine residue was replaced by arginine (Arginine, R) using site-directed mutagenesis. The following primer sets were used for PCR to prepare the substituted plasmid DNAs.

(Ghrelin K39R FP)
(SEQ ID NO: 84)
5′-AGTCCAGCAGAGAAGGGAGTCGAAGAAGCCA-3′,
RP
(SEQ ID NO: 85)
5′-TGGCTTCTTCGACTCCCT TCTCTGCTGGACT-3′;
(Ghrelin K42R)
FP
(SEQ ID NO: 86)
5′-AGAAAGGAGTCGAGGAAGCCACCAGCCAAGC-3′,
RP
(SEQ ID NO: 87)
5′-GCT TGGCTGGTGGCTTCCTCGACTCCTTTCT-3′;
(Ghrelin K43R FP)
(SEQ ID NO: 88)
5′-AGAAAGGAGTCGAAGAGGCCACCAGC CAAGC-3′,
RP
(SEQ ID NO: 89)
5′-GCTTGGCTGGTGGCCTCTTCGACTCCTTTCT-3′;
and
(Ghrelin K47R)
FP
(SEQ ID NO: 90)
5′-AAGAAGCCACC AGCCAGGCTGCAGCCCCGA-3′,
RP
(SEQ ID NO: 91)
5′-TCGGGGCTGCAGCCTGGCTGGTGGCTTCTT-3′.

Four plasmid DNAs each of which one or more lysine residues were replaced with arginine (K→R) were prepared by using pcDNA3-myc-Ghrelin as a template (Table 13).

TABLE 13

| Lysine(K) residue site | Ghrelin construct, replacement of K with R |
|---|---|
| 39 | pcDNA3-myc-Ghrelin (K39R) |
| 42 | pcDNA3-myc-Ghrelin (K42R) |
| 43 | pcDNA3-myc-Ghrelin (K43R) |
| 47 | pcDNA3-myc-Ghrelin (K47R) |

2. In Vivo Ubiquitination Analysis

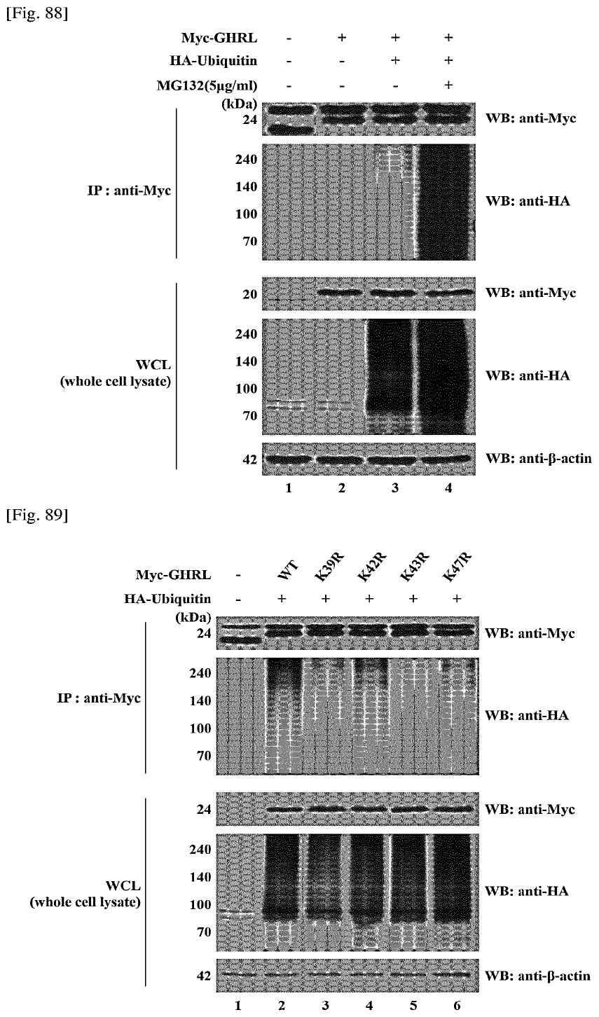

The HEK 293T cell was transfected with the plasmid encoding pcDNA3-myc-Ghrelin WT and pMT123-HA-ubiquitin. For the analysis of the ubiquitination level, pcDNA3-myc-Ghrelin WT 2 μg and pMT123-HA-ubiquitin DNA 1 μg were co-transfected into the cell. 24 hrs after the transfection, the cells were treated with MG132 (proteasome inhibitor, 5 μg/ml) for 6 hrs, thereafter immunoprecipitation analysis was carried out (FIG. 88). Then, the HEK 293T cells were transfected with the plasmids encoding pcDNA3-myc-Ghrelin WT, pcDNA3-myc-Ghrelin mutant (K39R), pcDNA3-myc-Ghrelin mutant (K42R), pcDNA3-myc-Ghrelin (K43R), pcDNA3-myc-Ghrelin mutant (K47R) and pMT123-HA-ubiquitin, respectively. For the analysis of the ubiquitination level, the cell was co-transfected with 1 μg of pMT123-HA-ubiquitin DNA and respective with 2 μg of pcDNA3-myc-Ghrelin WT, pcDNA3-myc-Ghrelin mutant (K39R), pcDNA3-myc-Ghrelin mutant (K42R), pcDNA3-myc-Ghrelin mutant (K43R) and pcDNA3-myc-Ghrelin mutant (K47R). Next, 24 hrs after the transfection, the immunoprecipitation was carried out (FIG. 89). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride)), and then was mixed with anti-myc (9E10) 1st antibody (sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution. The protein sample was separated by SDS-PAGE, after mixing with 2×SDS buffer and heating at 100° C., for 7 minutes. The separated proteins were moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), poly-ubiquitin chain was formed by the binding of the ubiquitin to pcDNA3-myc-Ghrelin WT, and thereby intense band indicating the presence of smear ubiquitin was detected (FIG. 88, lanes 3 and 4). Further, when the cells were treated with MG132 (proteasome inhibitor, 5 μg/ml) for 6 hrs, poly-ubiquitin chain formation was increased and thus the more intense band indicating ubiquitin was appeared (FIG. 88, lane 4). Further, as for the pcDNA3-myc-Ghrelin mutant (K39R), pcDNA3-myc-Ghrelin mutant (K42R), pcDNA3-myc-Ghrelin mutant (K43R) and pcDNA3-myc-Ghrelin mutant (K47R), the band was less intense than the wild type, and smaller amount of ubiquitin was detected since the mutants above were not bound to the ubiquitin (FIG. 89, lanes 3 to 6). These results represent that prepro-GHRL first binds to ubiquitin, and then is degraded through the polyubiquitin chain which is formed by ubiquitin-proteasome system.

3. Assessment of Ghrelin Half-Life Using Protein Synthesis Inhibitor Cycloheximide (CHX)

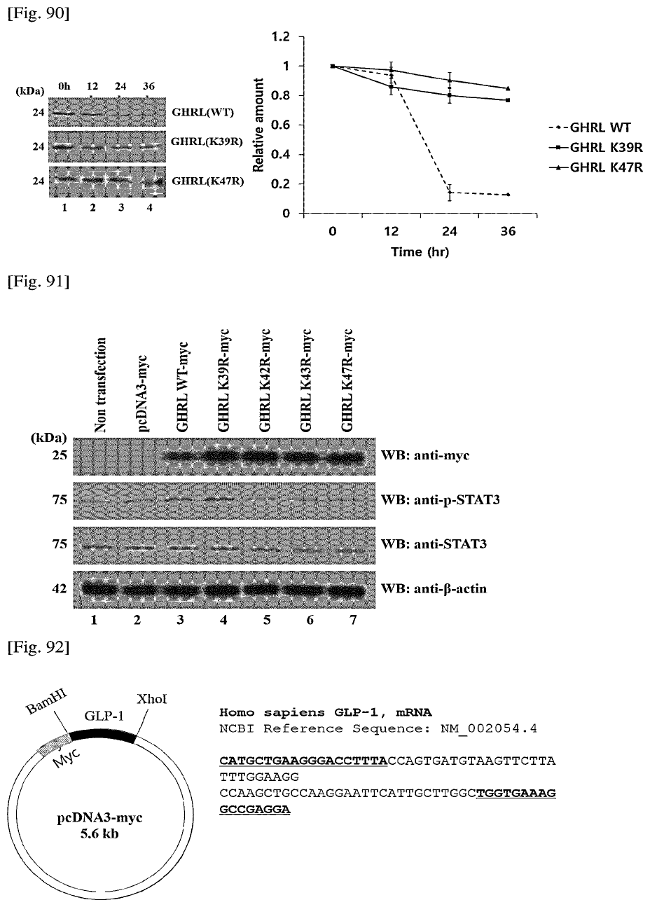

The HEK 293T cell was transfected with 2 μg of pcDNA3-myc-Ghrelin mutant (K39R), pcDNA3-myc-Ghrelin mutant (K42R), pcDNA3-myc-Ghrelin mutant (K43R) and pcDNA3-myc-Ghrelin mutant (K47R), respectively. 48 hrs after the transfection, the cells were treated with the protein synthesis inhibitor, cyclohexamide (CHX) (Sigma-Aldrich) (100 μg/ml), and then the half-life of each protein was detected for 12, 24 and 36 hrs after the treatment of the inhibitor. As a result, the degradation of human Ghrelin was observed (FIG. 90). The half-life of human Ghrelin was less than 15 hrs, while the half-lives of human pcDNA3-myc-Ghrelin mutant (K39R), pcDNA3-myc-Ghrelin mutant (K42R), pcDNA3-myc-Ghrelin mutant (K43R) and pcDNA3-myc-Ghrelin (K47R) were prolonged to 36 hrs or more, as shown in FIG. 90.

4. Signal Transduction by Ghrelin and the Substituted Ghrelin in Cells

It was reported that appetite stimulating hormone regulates cell growth via the growth hormone secretagogue receptor (GHS-R), and increases STAT3 through in vivo calcium regulation (Mol Cell Endocrinol., 285, 19-25, 2008). In this experiment, we examined the signal transduction by Ghrelin and the substituted Ghrelin in cells. First, the HepG2 cell (ATCC, AB-8065) was starved for 8 hrs, and then transfected by using 3 μg of pcDNA3-myc-Ghrelin WT, pcDNA3-myc-Ghrelin mutant (K39R), pcDNA3-myc-Ghrelin mutant (K42R) and pcDNA3-myc-Ghrelin mutant (K43R) and pcDNA3-myc-Ghrelin mutant (K47R), respectively. 2 days after the transfection, the proteins were extracted from the cells and quantified. Western blot was performed to analyze the signal transduction in the cells. The proteins separated from the HepG2 cell transfected with respective pcDNA3-myc-Ghrelin WT, pcDNA3-myc-Ghrelin mutant (K39R), pcDNA3-myc-Ghrelin mutant (K42R), pcDNA3-myc-Ghrelin mutant (K43R) and pcDNA3-myc-Ghrelin mutant (K47R) were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-myc (9E10, Santa Cruz Biotechnology, sc-40), anti-STAT3 (sc-21876), anti-phospho-STAT3 (Y705, cell signaling 9131S) and anti-β-actin (sc-47778) in 1:1,000 (w/w). As a result, pcDNA3-myc-Ghrelin mutant (K39R) showed the same or increased phospho-STAT3 signal transduction in HepG2 cell, in comparison to the wild type (FIG. 91).

### Example 14: The Analysis of Ubiquitination and Half-Life Increase of Glucagon-Like Peptide-1 (GLP-1), and the Analysis of Signal Transduction in Cells

1. Glucagon-Like Peptide-1 (GLP-1) Expression Vector Cloning and Protein Expression

(1) Glucagon-Like Peptide-1 (GLP-1) Expression Vector Cloning

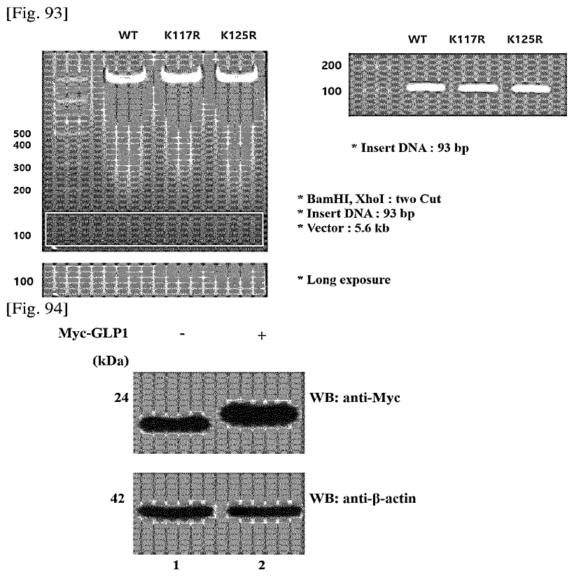

The glucagon-like peptide-1 (GLP-1) DNA amplified by PCR was treated with EcoRI, and then ligated to pcDNA3-myc vector (5.6 kb) previously digested with the same enzyme (FIG. 92, GLP-1 amino acid sequence: SEQ ID NO: 92). Then, agarose gel electrophoresis was carried out to confirm the presence of the DNA insert, after restriction enzyme digestion of the cloned vector (FIG. 93). The nucleotide sequences shown in underlined bold letters in FIG. 92 indicate the primer sets used for the PCR to confirm the cloned sites (FIG. 93). The PCR conditions are as follows: Step 1: at 94° C. for 3 minutes (1 cycle); Step 2: at 94° C. for 30 seconds; at 58° C. for 30 seconds; at 72° C. for 20 seconds (25 cycles); and Step 3: at 72° C. for 10 minutes (1 cycle), and then held at 4° C. For the assessment of the expression of proteins encoded by cloned DNA, western blot was carried out with anti-myc antibody (9E10, sc-40) to myc of pcDNA3-myc vector shown in the map of FIG. 92. The western blot result showed that the GLP-1 bound to myc was expressed well. The normalization with actin assured that proper amount of protein was loaded (FIG. 94).

(2) Lysine (Lysine. K) Residue Substitution

Lysine residue was replaced with arginine (Arginine, R) using site-directed mutagenesis. The following primer sets were used for PCR to prepare the substituted plasmid DNAs.

(GLP-1 K117R)
FP
(SEQ ID NO: 93)
5′-AAGCTGCCAGGGAATTCA-3′,
RP
(SEQ ID NO: 94)
5′-TGAATTCCCTGGCAGCTT-3′;
and
(GLP-1 K125R)
FP
(SEQ ID NO: 95)
5′-TTGGCTGGTGAGAGGCC-3′,
RP
(SEQ ID NO: 96)
5′-GGCCTCTCACCAGCCAA-3′.

Two plasmid DNAs each of which one or more lysine residues were replaced by arginine (K→R) were produced by using pcDNA3-myc-GLP-1 as a template (Table 15).

TABLE 15

| Lysine(K) residue site | GLP-1 construct, replacement of K with R |
|---|---|
| 117 | pcDNA3-myc-GLP-1 (K117R) |
| 125 | pcDNA3-myc-GLP-1 (K125R) |

2. In Vivo Ubiquitination Analysis

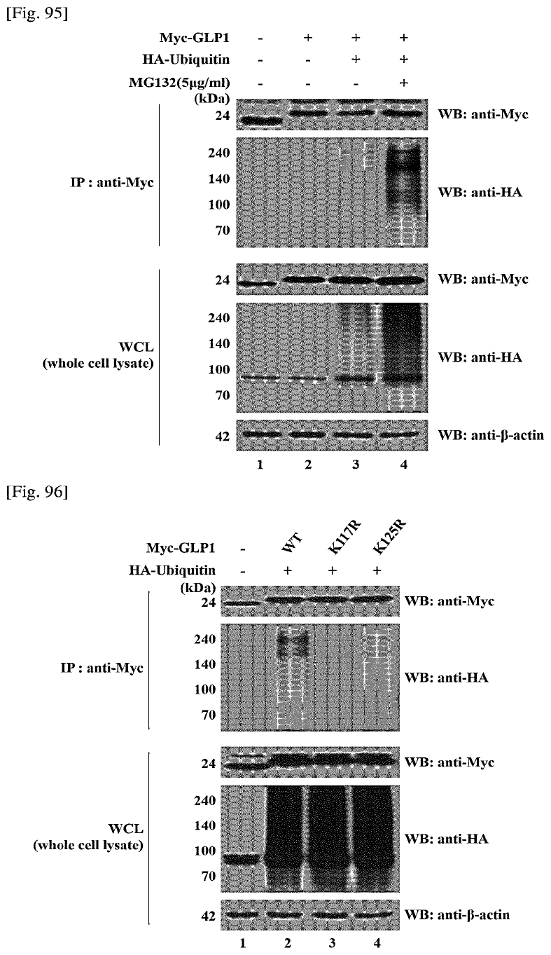

The HEK 293T cell was transfected with the plasmid encoding pcDNA3-myc-GLP-1 WT and pMT123-HA-ubiquitin. For the analysis of the ubiquitination level, pcDNA3-myc-GLP-1 WT 2 μg and pMT123-HA-ubiquitin DNA 1 μg were co-transfected into the cells. 24 hrs after the transfection, the cells were treated with MG132 (proteasome inhibitor, 5 μg/ml) for 6 hrs, thereafter immunoprecipitation analysis was carried out (FIG. 95). Then, the HEK 293T cells were transfected with the plasmids encoding pcDNA3-myc-GLP-1 WT, pcDNA3-myc-GLP-1 mutant (K117R), pcDNA3-myc-GLP-1 mutant (K125R) and pMT123-HA-ubiquitin, respectively. For the analysis of the ubiquitination level, the cells were co-transfected with 1 μg of pMT123-HA-ubiquitin DNA, and with respective 2 μg of pcDNA3-myc-GLP-1 WT, pcDNA3-myc-GLP-1 mutant (K117R) and pcDNA3-myc-GLP-1 mutant (K125R). Next, 24 hrs after the transfection, immunoprecipitation was carried out (FIG. 96). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride)), and then was mixed with anti-myc (9E10) 1st antibody (sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution. The protein sample was separated by SDS-PAGE, after mixing with 2×SDS buffer and heating at 100° C., for 7 minutes. The separated proteins were moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), poly-ubiquitin chain was formed by the binding of the ubiquitin to pcDNA3-myc-GLP-1 WT, and thereby intense band indicating the presence of smear ubiquitin was detected (FIG. 95, lanes 3 and 4). Further, when the cells were treated with MG132 (proteasome inhibitor, 5 μg/ml) for 6 hrs, poly-ubiquitin chain formation was increased and thus the more intense band indicating ubiquitin was appeared (FIG. 95, lane 4). Further, as for the pcDNA3-myc-GLP-1 mutant (K117R) and pcDNA3-myc-GLP-1 mutant (K125R), the band was less intense than the wild type, and smaller amount of ubiquitin was detected since the mutants above were not bound to the ubiquitin (FIG. 96, lanes 3 and 4). These results represent that GLP-1 first binds to ubiquitin, and then is degraded through the polyubiquitin chain which is formed by ubiquitin-proteasome system.

3. Assessment of GLP-1 Half-Life Using Protein Synthesis Inhibitor Cyclohexamide (CHX)

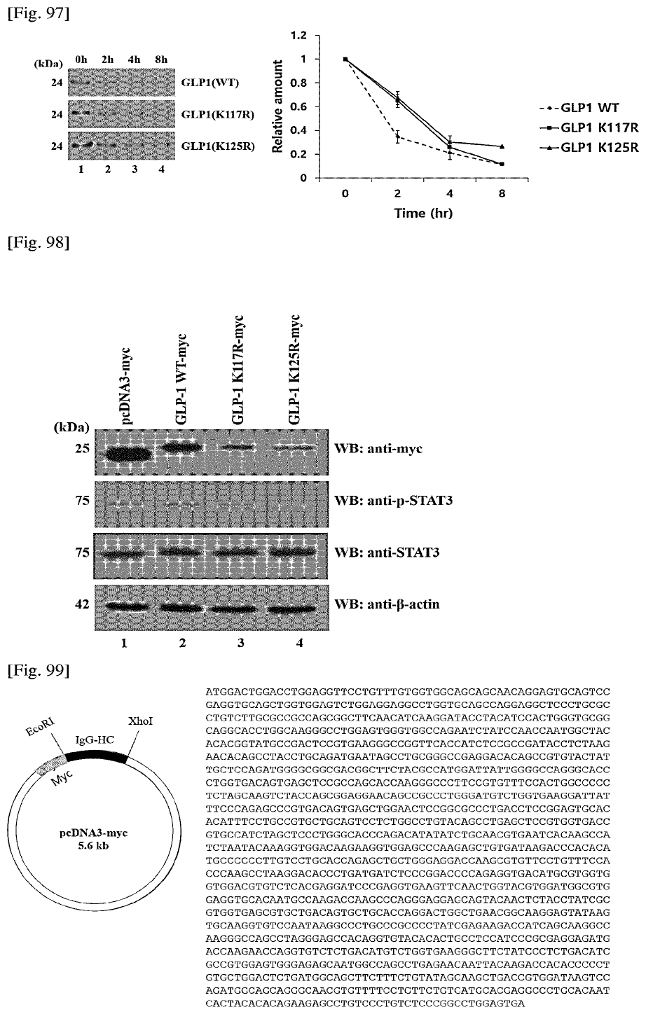

The HEK 293T cell was transfected with 2 μg of pcDNA3-myc-GLP-1 WT, pcDNA3-myc-GLP-1 mutant (K117R) and pcDNA3-myc-GLP-1 mutant (K125R), respectively. 48 hrs after the transfection, the cells were treated with the protein synthesis inhibitor, cyclohexamide (CHX) (Sigma-Aldrich) (100 μg/ml), and then the half-life of each protein was detected for 2, 4 and 8 hrs after the treatment of the inhibitor. As a result, the degradation of human GLP-1 was observed (FIG. 97). The half-life of human GLP-1 was about 2 hrs, while the half-lives of human pcDNA3-myc-GLP-1 mutant (K117R) and pcDNA3-myc-GLP-1 mutant (K125R) were prolonged to 4 hrs or more, as shown in FIG. 97.

4. Examination of Signal Transduction by GLP-1 and the Substituted GLP-1 in Cells

The GLP-1 regulates glucose homeostasis and improves insulin sensitivity, and thus it can be used for treating diabetes and induce STAT3 activity (Biochem Biophys Res Commun., 425(2), 304-308, 2012). In this experiment, we examined the signal transduction by GLP-1 and the substituted GLP-1 in cells. First, the HepG2 cell was starved for 8 hrs, and then transfected by using 6 kg of pcDNA3-myc-GLP-1 WT, pcDNA3-myc-GLP-1 mutant (K117R) and pcDNA3-myc-GLP-1 mutant (K125R), respectively. 2 days after the transfection, the proteins were extracted from the cells and quantified. Western blot was performed to analyze the signal transduction in the cells. The proteins separated from the HepG2 cell transfected with respective pcDNA3-myc-GLP-1 WT, pcDNA3-myc-GLP-1 mutant (K117R) and pcDNA3-myc-GLP-1 mutant (K125R) were moved to PVDF membrane. Then, the proteins were developed with ECL system using anti-rabbit (goat anti-rabbit IgG-HRP, Santa Cruz Biotechnology, sc-2004) and anti-mouse (Peroxidase-labeled antibody to mouse IgG (H+L), KPL, 074-1806) secondary antibodies and blocking solution which comprises anti-myc (9E10, Santa Cruz Biotechnology, sc-40), anti-STAT3 (sc-21876), anti-phospho-STAT3 (Y705, cell signaling 9131S) and anti-β-actin (sc-47778) in 1:1,000 (w/w). As a result, pcDNA3-myc-GLP-1 mutant (K117R) showed the same or increased phospho-STAT3 signal transduction in HepG2 cells, in comparison to the wild type (FIG. 98)

### Example 15: The Analysis of Ubiquitination and Half-Life Increase of IgG Heavy Chain, and the Analysis of Signal Transduction in Cells

1. IgG Heavy Chain Expression Vector Cloning and Protein Expression

(1) IgG Heavy Chain Expression Vector Cloning

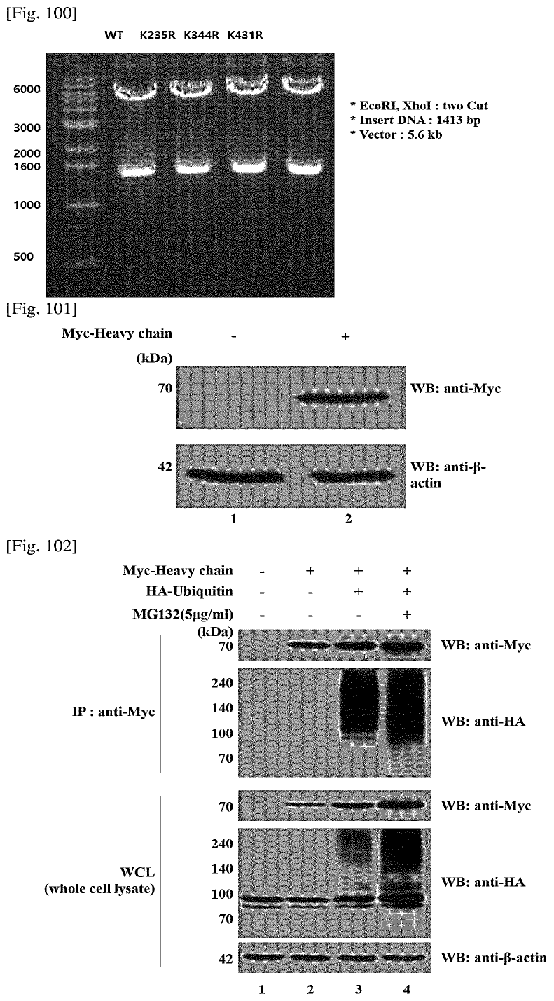

The IgG heavy chain (HC) DNA sequence was synthesized in accordance with the description of Roche's EP1308455 B9 (A composition comprising anti-HER2 antibodies, p. 24), and further optimized to express well in a mammalian cell. Then, IgG heavy chain (HC) DNA amplified by PCR was treated with EcoRI and XhoI, and then ligated to pcDNA3-myc vector (5.6 kb) previously digested with the same enzyme (FIG. 99, IgG heavy chain amino acid sequence: SEQ ID NO: 97). Then, agarose gel electrophoresis was carried out to confirm the presence of the DNA insert, after restriction enzyme digestion of the cloned vector (FIG. 100). For the assessment of the expression of proteins encoded by cloned DNA, western blot was carried out with anti-myc antibody (9E10, sc-40) to myc of pcDNA3-myc vector shown in the map of FIG. 99. The western blot result showed that the IgG heavy chain (HC) bound to myc was expressed well. The normalization with actin assured that proper amount of protein was loaded (FIG. 101).

(2) Lysine (Lysine, K) Residue Substitution

Lysine residue was replaced with arginine (Arginine, R) using site-directed mutagenesis. The following primer sets were used for PCR to prepare the substituted plasmid DNAs.

(IgG HC K235R)
FP
(SEQ ID NO: 98)
5′-ACAAAGGTGGACAGGAAGGTGGAGCCCAAG-3′,
RP
(SEQ ID NO: 99)
5′-CTTGGGCTCCACCTTCC TGTCCACCTTTGT-3′;
(IgG HC K344R)
FP
(SEQ ID NO: 100)
5′-GAGTATAAGTGCAGGGTGTCCAATAAGGCCCTGC-3′,
RP
(SEQ ID NO: 101)
5′-GCAGGGCCTTATTGGACACCCTGCACTTATACTC-3′;
and
(IgG HC K431R)
FP
(SEQ ID NO: 102)
5′-CTTTCTGTATAGCAGGCTGA CCGTGGATAAGTCC-3′,
RP
(SEQ ID NO: 103)
5′-GGACTTATCCACGGTCAGCCTGCTATACAGAAAG-3′.

Three plasmid DNAs each of which one or more lysine residues were replaced with arginine (K→R) were prepared by using pcDNA3-myc-IgG HC DNA as a template (Table 14).

TABLE 14

| Lysine(K) residue site | IgG HC construct, replacement of K with R |
|---|---|
| 235 | pcDNA3-myc-IgG HC (K235R) |
| 344 | pcDNA3-myc-IgG HC (K344R) |
| 431 | pcDNA3-myc-IgG HC (K431R) |

2. In Vivo Ubiquitination Analysis

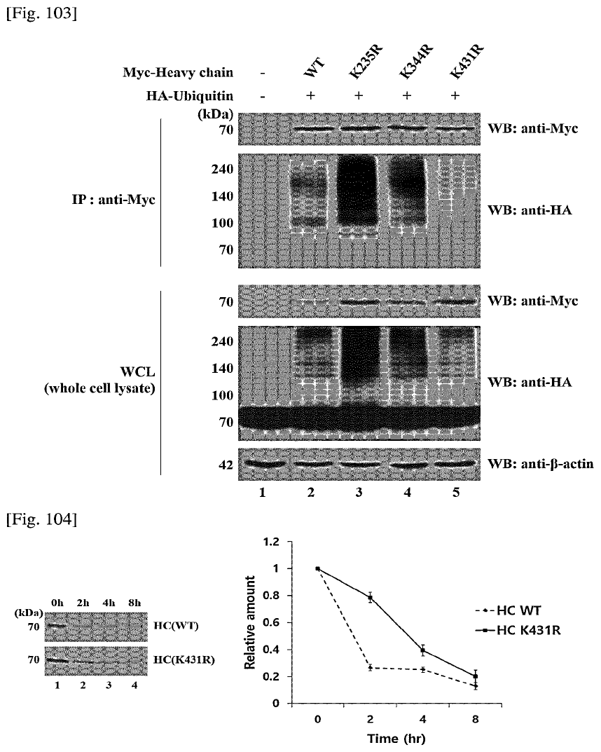

The HEK 293T cell was transfected with the plasmid encoding pcDNA3-myc-IgG-HC WT and pMT123-HA-ubiquitin. For the analysis of the ubiquitination level, pcDNA3-myc-IgG-HC WT 2 μg and pMT123-HA-ubiquitin DNA 1 μg were co-transfected into the cells. 24 hrs after the transfection, the cells were treated with MG132 (proteasome inhibitor, 5 μg/ml) for 6 hrs, thereafter immunoprecipitation analysis was carried out (FIG. 102). Then, the HEK 293T cell was transfected with the plasmids encoding pcDNA3-myc-IgG-HC WT, pcDNA3-myc-IgG-HC mutant (K235R), pcDNA3-myc-IgG-HC mutant (K344R), pcDNA3-myc-IgG-HC mutant (K431R) and pMT123-HA-ubiquitin, respectively. For the analysis of the ubiquitination level, the cells were co-transfected with 1 μg of pMT123-HA-ubiquitin DNA, and with respective 2 μg of pcDNA3-myc-IgG-HC WT, pcDNA3-myc-IgG-HC mutant (K235R), pcDNA3-myc-IgG-HC mutant (K344R) and pcDNA3-myc-IgG-HC mutant (K431R). Next, 24 hrs after the transfection, immunoprecipitation was carried out (FIG. 103). The sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution. The protein sample was separated by SDS-PAGE, after mixing with 2×SDS buffer and heating at 100° C., for 7 minutes.

The separated protein was moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), poly-ubiquitin chain was formed by the binding of the ubiquitin to pcDNA3-myc-IgG-HC WT, and thereby intense band indicating the presence of smear ubiquitin was detected (FIG. 102, lanes 3 and 4). Further, when the cells were treated with MG132 (proteasome inhibitor, 5 μg/ml) for 6 hrs, poly-ubiquitin chain formation was increased and thus the more intense band indicating ubiquitin was appeared (FIG. 102, lane 4). Further, as for the pcDNA3-myc-IgG-HC mutant (K431R), the band was less intense than the wild type, and smaller amount of ubiquitin was detected since the mutant above was not bound to the ubiquitin (FIG. 103, lane 5). These results represent that IgG-HC first binds to ubiquitin, and then is degraded through the polyubiquitin chain which is formed by ubiquitin-proteasome system.

3. Assessment of IgG-HC Half-Life Using Protein Synthesis Inhibitor Cyclohexamide (CHX)

The HEK 293T cell was transfected with 2 μg of pcDNA3-myc-IgG-HC WT, pcDNA3-myc-IgG-HC mutant (K235R), pcDNA3-myc-IgG-HC mutant (K344R) and pcDNA3-myc-IgG-HC mutant (K431R), respectively. 48 hrs after the transfection, the cells were treated with the protein synthesis inhibitor, cyclohexamide (CHX) (Sigma-Aldrich) (100 μg/ml), and then the half-life of each protein was detected for 2, 4 and 8 hrs after the treatment of the inhibitor. As a result, the suppression of degradation of human IgG-HC was observed (FIG. 104). The half-life of human IgG-HC was less than 2 hrs, while the half-life of human pcDNA3-myc-IgG-HC mutant (K431R) was prolonged to 4 hrs or more, as shown in FIG. 104.

### Example 16: The Analysis of Ubiquitination and Half-Life Increase of IgG Light Chain (LC), and the Analysis of Signal Transduction in Cells

1. IgG Light Chain (LC) Expression Vector Cloning and Protein Expression

(1) IgG Light Chain (LC) Expression Vector Cloning

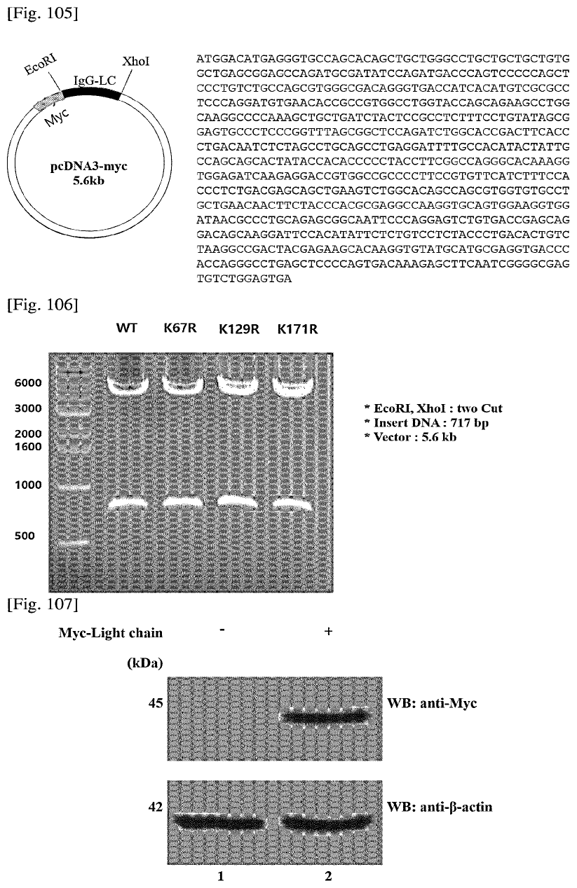

The IgG light chain (LC) DNA sequence was synthesized in accordance with the description of Roche's EP1308455 B9 (A composition comprising anti-HER2 antibodies, p. 23), and further optimized to express well in a mammalian cell. Then, IgG light chain (LC) DNA amplified by PCR was treated with EcoRI and XhoI, and then ligated to pcDNA3-myc vector (5.6 kb) previously digested with the same enzyme (FIG. 105, IgG light chain amino acid sequence: SEQ ID NO: 104). Then, agarose gel electrophoresis was carried out to confirm the presence of the DNA insert, after restriction enzyme digestion of the cloned vector (FIG. 106). For the assessment of the expression of proteins encoded by cloned DNA, western blot was carried out with anti-myc antibody (9E10, sc-40) to myc of pcDNA3-myc vector shown in the map of FIG. 105. The western blot result showed that the IgG light chain (LC) bound to myc was expressed well. The normalization with actin assured that proper amount of protein was loaded (FIG. 107).

(2) Lysine (Lysine. K) Residue Substitution

Lysine residue was replaced with arginine (Arginine, R) using site-directed mutagenesis. The following primer sets were used for PCR to prepare the substituted plasmid DNAs.

(IgG LC K67R)
FP
(SEQ ID NO: 105)
5′-CCTGGCAAGGCCCCAAGGCTGCTGATCTAC-3′,
RP
(SEQ ID NO: 106)
5′-GTAGATCAGCAGCCTTGGGGCCTTGCCAGG-3′;
(IgG LC K129R)
FP
(SEQ ID NO: 107)
5′-ACAAAGGTGGAGATCAGGAGGACCGTGGCC-3′,
RP
(SEQ ID NO: 108)
5′-GGCCACGGTCCTCCTGATCTCCACCTTTGT-3′;
and
(IgG LC K171R)
FP
(SEQ ID NO: 109)
5′-GCCAAGGTGCAGTGGAGGGTGGATAACGCC-3′,
RP
(SEQ ID NO: 110)
5′-GGCGTTATCCACCCTCCACTGCACCTTGGC-3′.

Three plasmid DNAs each of which one or more lysine residues were replaced with arginine (K→R) were prepared by using pcDNA3-myc-IgG LC DNA as a template (Table 16).

TABLE 16

| Lysine(K) residue site | IgG LC construct, replacement of K with R |
|---|---|
| 67 | pcDNA3-myc-IgG LC (K67R) |
| 129 | pcDNA3-myc-IgG LC (K129R) |
| 171 | pcDNA3-myc-IgG LC (K171R) |

2. In Vivo Ubiquitination Analysis

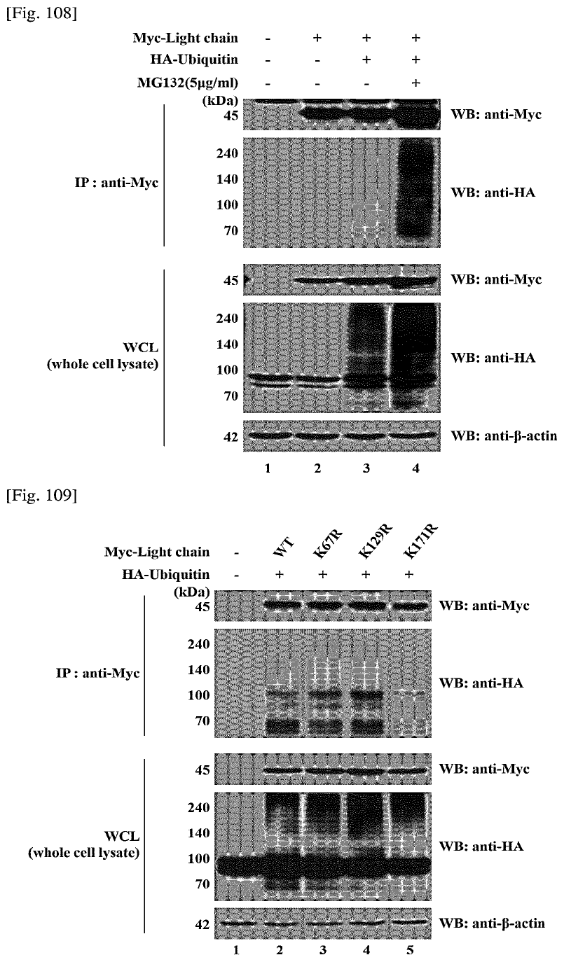

The HEK 293T cell was transfected with the plasmid encoding pcDNA3.1-6myc-IgG-LC WT and pMT123-HA-ubiquitin. For the analysis of the ubiquitination level, pcDNA3-myc-IgG-LC WT 2 μg and pMT123-HA-ubiquitin DNA 1 μg were co-transfected into the cells. 24 hrs after the transfection, the cells were treated with MG132 (proteasome inhibitor, 5 μg/ml) for 6 hrs, thereafter immunoprecipitation analysis was carried out (FIG. 108). Then, the HEK 293T cells were transfected with the plasmids encoding pcDNA3-myc-IgG-LC WT, pcDNA3-myc-IgG-LC mutant (K67R), pcDNA3-myc-IgG-LC mutant (K129R), pcDNA3-myc-IgG-LC mutant (K171R) and pMT123-HA-ubiquitin, respectively. For the analysis of the ubiquitination level, the cells were co-transfected with 1 μg of pMT123-HA-ubiquitin DNA, and with respective 2 μg of pcDNA3-myc-IgG-LC WT, pcDNA3-myc-IgG-LC mutant (K67R), pcDNA3-myc-IgG-LC mutant (K129R) and pcDNA3-myc-IgG-LC mutant (K171R). Next, 24 hrs after the transfection, the immunoprecipitation was carried out (FIG. 109). The protein sample obtained for the immunoprecipitation was dissolved in buffering solution comprising (1% Triton X, 150 mM NaCl, 50 mM Tris-HCl, pH 8 and 1 mM PMSF (phenylmethanesulfonyl fluoride)), and then was mixed with anti-myc (9E10) 1st antibody (Santa Cruz Biotechnology, sc-40). Thereafter, the mixture was incubated at 4° C., overnight. The immunoprecipitant was separated, following the reaction with A/G bead (Santa Cruz Biotechnology) at 4° C., for 2 hrs. Subsequently, the separated immunoprecipitant was washed twice with buffering solution. The protein sample was separated by SDS-PAGE, after mixing with 2×SDS buffer and heating at 100° C., for 7 minutes. The separated proteins were moved to polyvinylidene difluoride (PVDF) membrane, and then developed with ECL system using anti-mouse (Peroxidase-labeled antibody to mouse IgG (H+L), KPL, 074-1806) secondary antibody and blocking solution which comprises anti-myc (9E10, sc-40), anti-HA (sc-7392) and anti-β-actin (sc-47778) in 1:1,000 (w/w). As a result, when immunoprecipitation was performed by using anti-myc (9E10, sc-40), poly-ubiquitin chain was formed by the binding of the ubiquitin to pcDNA3-myc-IgG-LC WT, and thereby intense band indicating the presence of smear ubiquitin was detected (FIG. 108, lanes 3 and 4). Further, when the cells were treated with MG132 (proteasome inhibitor, 5 μg/ml) for 6 hrs, poly-ubiquitin chain formation was increased and thus the more intense band indicating ubiquitin was appeared (FIG. 108, lane 4). Further, as for the pcDNA3-myc-IgG-LC mutant (K171R), the band was less intense than the wild type, and smaller amount of ubiquitin was detected since the mutants above were not bound to the ubiquitin (FIG. 109, lane 5). These results represent that IgG-LC first binds to ubiquitin, and then is degraded through the polyubiquitin chain which is formed by ubiquitin-proteasome system.

3. Assessment of IgG-LC Half-Life Using Protein Synthesis Inhibitor Cycloheximide (CHX)

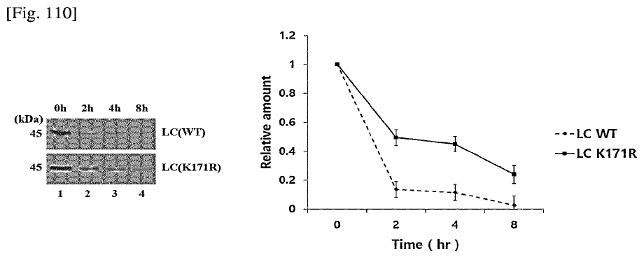

The HEK 293T cell was transfected with 2 μg of pcDNA3-myc-IgG-LC WT, pcDNA3-myc-IgG-LC mutant (K67R), pcDNA3-myc-IgG-LC mutant (K129R) and pcDNA3-myc-IgG-LC mutant (K171R), respectively. 48 hrs after the transfection, the cells were treated with the protein synthesis inhibitor, cyclohexamide (CHX) (Sigma-Aldrich) (100 μg/ml), and then the half-life of the proteins was detected for 2, 4 and 8 hrs after the treatment of the inhibitor. As a result, the degradation of the substituted human IgG-LC of the present invention was suppressed (FIG. 110). The half-life of human IgG-LC was less than 1 hr, while the half-life of human pcDNA3-myc-IgG-LC mutant (K171R) was prolonged to 2 hrs or more, as shown in FIG. 110.

### INDUSTRIAL APPLICABILITY

The present invention would be used to develop a protein or (poly)peptide therapeutic agents, since the mutated proteins of the invention have prolonged half-life.

1. An interferon-β having a prolonged half-life, wherein the interferon-β has amino acid sequences of SEQ ID NO: 36, and one or more lysine residue(s) at positions corresponding to 40, 126, and 155 from the N-terminus of the interferon-β are replaced by arginine(s).

2. A pharmaceutical composition for preventing and/or treating multiple sclerosis, autoimmune disease, rheumatoid arthritis; or cancer comprising solid cancer and blood cancer; or infectious disease comprising virus infection, HIV related disease and Hepatitis C, which comprises the interferon-β of claim 1, and pharmaceutically accepted excipient.

3. An expression vector comprising: (a) promoter; (b) a nucleic acid sequence encoding the interferon-β of claim 1; and optionally a linker, wherein the promoter and the nucleic acid sequence and are operably linked.

4. A host cell comprising the expression vector of claim 3.


SEQUENCE LISTING (126 sequences)

SEQ ID NO: 1 - protein, 198 aa, synthetic construct
  REGION 1..198: note = Description of Artificial Sequence: Synthetic polypeptide
  REGION 1..198: note = Human beta trophin
MPVPALCLLWALAMVTRPASAAPMGGPELAQHEELTLLFHGTLQLGQALNGVYRTTEGRLTKARNSLGLYGRTIELLGQEVSRGRDAAQELRASLLETQMEEDILQLQAEATAEVLGEVAQAQKVLRDSVQRLEVQLRSAWLGPAYREFEVLKAHADKQSHILWALTGHVQRQRREMVAQQHRLRQIQERLHTAALPA

SEQ ID NO: 2 - DNA, 25 bp, synthetic construct
  misc_feature 1..25: note = Description of Artificial Sequence: Synthetic oligonucleotide
  misc_feature 1..25: note = Human beta trophin
  source 1..25: mol_type = other DNA
agggacggctgacaagggccaggaa

SEQ ID NO: 3 - DNA, 26 bp, synthetic construct
  misc_feature 1..26: note = Description of Artificial Sequence: Synthetic oligonucleotide
  misc_feature 1..26: note = Human beta trophin
  source 1..26: mol_type = other DNA
ccaggctgttcctggcccttgtcagc

SEQ ID NO: 4 - DNA, 25 bp, synthetic construct
  misc_feature 1..25: note = Description of Artificial Sequence: Synthetic oligonucleotide
  misc_feature 1..25: note = Human beta trophin
  source 1..25: mol_type = other DNA
ggcacagagggtgctacgggacagc

SEQ ID NO: 5 - DNA, 25 bp, synthetic construct
  misc_feature 1..25: note = Description of Artificial Sequence: Synthetic oligonucleotide
  misc_feature 1..25: note = Human beta trophin
  source 1..25: mol_type = other DNA
cgtagcaccctctgtgcctgggcca

SEQ ID NO: 6 - DNA, 26 bp, synthetic construct
  misc_feature 1..26: note = Description of Artificial Sequence: Synthetic oligonucleotide
  misc_feature 1..26: note = Human beta trophin
  source 1..26: mol_type = other DNA
gaatttgaggtcttaagggctcacgc

SEQ ID NO: 7 - DNA, 27 bp, synthetic construct
  misc_feature 1..27: note = Description of Artificial Sequence: Synthetic oligonucleotide
  misc_feature 1..27: note = Human beta trophin
  source 1..27: mol_type = other DNA
cttgtcagcgtgagcccttaagacctc

SEQ ID NO: 8 - DNA, 26 bp, synthetic construct
  misc_feature 1..26: note = Description of Artificial Sequence: Synthetic oligonucleotide
  misc_feature 1..26: note = Human beta trophin
  source 1..26: mol_type = other DNA
gctcacgctgacaggcagagccacat

SEQ ID NO: 9 - DNA, 27 bp, synthetic construct
  misc_feature 1..27: note = Description of Artificial Sequence: Synthetic oligonucleotide
  misc_feature 1..27: note = Human beta trophin
  source 1..27: mol_type = other DNA
ccataggatgtggctctgcctgtcagc

SEQ ID NO: 10 - protein, 217 aa, synthetic construct
  REGION 1..217: note = Description of Artificial Sequence: Synthetic polypeptide
  REGION 1..217: note = Human growth hormone
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 11 - DNA, 24 bp, synthetic construct
  misc_feature 1..24: note = Description of Artificial Sequence: Synthetic oligonucleotide
  misc_feature 1..24: note = Human growth hormone
  source 1..24: mol_type = other DNA
ccaaaggaacagaggtattcattc

SEQ ID NO: 12 - DNA, 24 bp, synthetic construct
  misc_feature 1..24: note = Description of Artificial Sequence: Synthetic oligonucleotide
  misc_feature 1..24: note = Human growth hormone
  source 1..24: mol_type = other DNA
caggaatgaatacctctgttcctt

SEQ ID NO: 13 - DNA, 21 bp, synthetic construct
  misc_feature 1..21: note = Description of Artificial Sequence: Synthetic oligonucleotide
  misc_feature 1..21: note = Human growth hormone
  source 1..21: mol_type = other DNA
gacctcctaagggacctagag

SEQ ID NO: 14 - DNA, 21 bp, synthetic construct
  misc_feature 1..21: note = Description of Artificial Sequence: Synthetic oligonucleotide
  misc_feature 1..21: note = Human growth hormone
  source 1..21: mol_type = other DNA
ctctaggtcccttaggaggtc

SEQ ID NO: 15 - DNA, 21 bp, synthetic construct
  misc_feature 1..21: note = Description of Artificial Sequence: Synthetic oligonucleotide
  misc_feature 1..21: note = Human growth hormone
  source 1..21: mol_type = other DNA
cagatcttcaggcagacctac

SEQ ID NO: 16 - DNA, 21 bp, synthetic construct
  misc_feature 1..21: note = Description of Artificial Sequence: Synthetic oligonucleotide
  misc_feature 1..21: note = Human growth hormone
  source 1..21: mol_type = other DNA
gtaggtctgcctgaagatctg

SEQ ID NO: 17 - protein, 110 aa, synthetic construct
  REGION 1..110: note = Description of Artificial Sequence: Synthetic polypeptide
  REGION 1..110: note = Human insulin
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 18 - DNA, 25 bp, synthetic construct
  misc_feature 1..25: note = Description of Artificial Sequence: Synthetic oligonucleotide
  misc_feature 1..25: note = Human insulin
  source 1..25: mol_type = other DNA
ggcttcttctacacacccaggaccc

SEQ ID NO: 19 - DNA, 24 bp, synthetic construct
  misc_feature 1..24: note = Description of Artificial Sequence: Synthetic oligonucleotide
  misc_feature 1..24: note = Human insulin
  source 1..24: mol_type = other DNA
ctcccggcgggtcctgggtgtgta

SEQ ID NO: 20 - DNA, 23 bp, synthetic construct
  misc_feature 1..23: note = Description of Artificial Sequence: Synthetic oligonucleotide
  misc_feature 1..23: note = Human insulin
  source 1..23: mol_type = other DNA
tccctgcagaggcgtggcattgt

SEQ ID NO: 21 - DNA, 27 bp, synthetic construct
  misc_feature 1..27: note = Description of Artificial Sequence: Synthetic oligonucleotide
  misc_feature 1..27: note = Human insulin
  source 1..27: mol_type = other DNA
ttgttccacaatgccacgcctctgcag

SEQ ID NO: 22 - protein, 188 aa, synthetic construct
  REGION 1..188: note = Description of Artificial Sequence: Synthetic polypeptide
  REGION 1..188: note = Human interferon alpha
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 23 - DNA, 21 bp, synthetic construct
  misc_feature 1..21: note = Description of Artificial Sequence: Synthetic oligonucleotide
  misc_feature 1..21: note = Human interferon alpha
  source 1..21: mol_type = other DNA
cttcagcacaagggactcatc

SEQ ID NO: 24 - DNA, 21 bp, synthetic construct
  misc_feature 1..21: note = Description of Artificial Sequence: Synthetic oligonucleotide
  misc_feature 1..21: note = Human interferon alpha
  source 1..21: mol_type = other DNA
cagatgagtcccttgtgctga

SEQ ID NO: 25 - DNA, 21 bp, synthetic construct
  misc_feature 1..21: note = Description of Artificial Sequence: Synthetic oligonucleotide
  misc_feature 1..21: note = Human interferon alpha
  source 1..21: mol_type = other DNA
ctcctagacagattctacact

SEQ ID NO: 26 - DNA, 21 bp, synthetic construct
  misc_feature 1..21: note = Description of Artificial Sequence: Synthetic oligonucleotide
  misc_feature 1..21: note = Human interferon alpha
  source 1..21: mol_type = other DNA
agtgtagaatctgtctaggag

SEQ ID NO: 27 - DNA, 21 bp, synthetic construct
  misc_feature 1..21: note = Description of Artificial Sequence: Synthetic oligonucleotide
  misc_feature 1..21: note = Human interferon alpha
  source 1..21: mol_type = other DNA
gctgtgaggagatacttccaa

SEQ ID NO: 28 - DNA, 21 bp, synthetic construct
  misc_feature 1..21: note = Description of Artificial Sequence: Synthetic oligonucleotide
  misc_feature 1..21: note = Human interferon alpha
  source 1..21: mol_type = other DNA
ttggaagtatctcctcacagc

SEQ ID NO: 29 - DNA, 21 bp, synthetic construct
  misc_feature 1..21: note = Description of Artificial Sequence: Synthetic oligonucleotide
  misc_feature 1..21: note = Human interferon alpha
  source 1..21: mol_type = other DNA
ctctatctgagagagaagaaa

SEQ ID NO: 30 - DNA, 21 bp, synthetic construct
  misc_feature 1..21: note = Description of Artificial Sequence: Synthetic oligonucleotide
  misc_feature 1..21: note = Human interferon alpha
  source 1..21: mol_type = other DNA
tttcttctctctcagatagag

SEQ ID NO: 31 - protein, 207 aa, synthetic construct
  REGION 1..207: note = Description of Artificial Sequence: Synthetic polypeptide
  REGION 1..207: note = Human G-CSF
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 32 - DNA, 24 bp, synthetic construct
  misc_feature 1..24: note = Description of Artificial Sequence: Synthetic oligonucleotide
  misc_feature 1..24: note = Human G-CSF
  source 1..24: mol_type = other DNA
agcttcctgctcaggtgcttagag

SEQ ID NO: 33 - DNA, 24 bp, synthetic construct
  misc_feature 1..24: note = Description of Artificial Sequence: Synthetic oligonucleotide
  misc_feature 1..24: note = Human G-CSF
  source 1..24: mol_type = other DNA
ttgctctaagcacctgagcaggaa

SEQ ID NO: 34 - DNA, 24 bp, synthetic construct
  misc_feature 1..24: note = Description of Artificial Sequence: Synthetic oligonucleotide
  misc_feature 1..24: note = Human G-CSF
  source 1..24: mol_type = other DNA
tgtgccacctacaggctgtgccac

SEQ ID NO: 35 - DNA, 24 bp, synthetic construct
  misc_feature 1..24: note = Description of Artificial Sequence: Synthetic oligonucleotide
  misc_feature 1..24: note = Human G-CSF
  source 1..24: mol_type = other DNA
ggggtggcacagcctgtaggtggc

SEQ ID NO: 36 - protein, 187 aa, synthetic construct
  REGION 1..187: note = Description of Artificial Sequence: Synthetic polypeptide
  REGION 1..187: note = Human interferon beta
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 37 - DNA, 21 bp, synthetic construct
  misc_feature 1..21: note = Description of Artificial Sequence: Synthetic oligonucleotide
  misc_feature 1..21: note = Human interferon beta
  source 1..21: mol_type = other DNA
cagtgtcagaggctcctgtgg

SEQ ID NO: 38 - DNA, 21 bp, synthetic construct
  misc_feature 1..21: note = Description of Artificial Sequence: Synthetic oligonucleotide
  misc_feature 1..21: note = Human interferon beta
  source 1..21: mol_type = other DNA
ccacaggagcctctgacactg

SEQ ID NO: 39 - DNA, 21 bp, synthetic construct
  misc_feature 1..21: note = Description of Artificial Sequence: Synthetic oligonucleotide
  misc_feature 1..21: note = Human interferon beta
  source 1..21: mol_type = other DNA
ctggaagaaagactggagaaa

SEQ ID NO: 40 - DNA, 21 bp, synthetic construct
  misc_feature 1..21: note = Description of Artificial Sequence: Synthetic oligonucleotide
  misc_feature 1..21: note = Human interferon beta
  source 1..21: mol_type = other DNA
tttctccagtctttcttccag

SEQ ID NO: 41 - DNA, 21 bp, synthetic construct
  misc_feature 1..21: note = Description of Artificial Sequence: Synthetic oligonucleotide
  misc_feature 1..21: note = Human interferon beta
  source 1..21: mol_type = other DNA
cattacctgagggccaaggag

SEQ ID NO: 42 - DNA, 21 bp, synthetic construct
  misc_feature 1..21: note = Description of Artificial Sequence: Synthetic oligonucleotide
  misc_feature 1..21: note = Human interferon beta
  source 1..21: mol_type = other DNA
ctccttggccctcaggtaatg

SEQ ID NO: 43 - protein, 193 aa, synthetic construct
  REGION 1..193: note = Description of Artificial Sequence: Synthetic polypeptide
  REGION 1..193: note = Human erythropoietin
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 44 - DNA, 24 bp, synthetic construct
  misc_feature 1..24: note = Description of Artificial Sequence: Synthetic oligonucleotide
  misc_feature 1..24: note = Human erythropoietin
  source 1..24: mol_type = other DNA
gcatgtggatagagccgtcagtgc

SEQ ID NO: 45 - DNA, 24 bp, synthetic construct
  misc_feature 1..24: note = Description of Artificial Sequence: Synthetic oligonucleotide
  misc_feature 1..24: note = Human erythropoietin
  source 1..24: mol_type = other DNA
gcactgacggctctatccacatgc

SEQ ID NO: 46 - DNA, 31 bp, synthetic construct
  misc_feature 1..31: note = Description of Artificial Sequence: Synthetic oligonucleotide
  misc_feature 1..31: note = Human erythropoietin
  source 1..31: mol_type = other DNA
tgacactttccgcagactcttccgagtctac

SEQ ID NO: 47 - DNA, 31 bp, synthetic construct
  misc_feature 1..31: note = Description of Artificial Sequence: Synthetic oligonucleotide
  misc_feature 1..31: note = Human erythropoietin
  source 1..31: mol_type = other DNA
gtagactcggaagagtctgcggaaagtgtca

SEQ ID NO: 48 - DNA, 21 bp, synthetic construct
  misc_feature 1..21: note = Description of Artificial Sequence: Synthetic oligonucleotide
  misc_feature 1..21: note = Human erythropoietin
  source 1..21: mol_type = other DNA
ctccggggaaggctgaagctg

SEQ ID NO: 49 - DNA, 21 bp, synthetic construct
  misc_feature 1..21: note = Description of Artificial Sequence: Synthetic oligonucleotide
  misc_feature 1..21: note = Human erythropoietin
  source 1..21: mol_type = other DNA
cagcttcagccttccccggag

SEQ ID NO: 50 - DNA, 23 bp, synthetic construct
  misc_feature 1..23: note = Description of Artificial Sequence: Synthetic oligonucleotide
  misc_feature 1..23: note = Human erythropoietin
  source 1..23: mol_type = other DNA
ggaaagctgaggctgtacacagg

SEQ ID NO: 51 - DNA, 23 bp, synthetic construct
  misc_feature 1..23: note = Description of Artificial Sequence: Synthetic oligonucleotide
  misc_feature 1..23: note = Human erythropoietin
  source 1..23: mol_type = other DNA
cctgtgtacagcctcagctttcc

SEQ ID NO: 52 - protein, 396 aa, synthetic construct
  REGION 1..396: note = Description of Artificial Sequence: Synthetic polypeptide
  REGION 1..396: note = Human bone morphogenetic protein-2
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 53 - DNA, 29 bp, synthetic construct
  misc_feature 1..29: note = Description of Artificial Sequence: Synthetic oligonucleotide
  misc_feature 1..29: note = Human bone morphogenetic protein-2
  source 1..29: mol_type = other DNA
gaaacgccttaggtccagctgtaagagac

SEQ ID NO: 54 - DNA, 29 bp, synthetic construct
  misc_feature 1..29: note = Description of Artificial Sequence: Synthetic oligonucleotide
  misc_feature 1..29: note = Human bone morphogenetic protein-2
  source 1..29: mol_type = other DNA
gtctcttacagctggacctaaggcgtttc

SEQ ID NO: 55 - DNA, 30 bp, synthetic construct
  misc_feature 1..30: note = Description of Artificial Sequence: Synthetic oligonucleotide
  misc_feature 1..30: note = Human bone morphogenetic protein-2
  source 1..30: mol_type = other DNA
ttaagtccagctgtaggagacaccctttgt

SEQ ID NO: 56 - DNA, 30 bp, synthetic construct
  misc_feature 1..30: note = Description of Artificial Sequence: Synthetic oligonucleotide
  misc_feature 1..30: note = Human bone morphogenetic protein-2
  source 1..30: mol_type = other DNA
acaaagggtgtctcctacagctggacttaa

SEQ ID NO: 57 - DNA, 23 bp, synthetic construct
  misc_feature 1..23: note = Description of Artificial Sequence: Synthetic oligonucleotide
  misc_feature 1..23: note = Human bone morphogenetic protein-2
  source 1..23: mol_type = other DNA
gttaactctaggattcctaaggc

SEQ ID NO: 58 - DNA, 23 bp, synthetic construct
  misc_feature 1..23: note = Description of Artificial Sequence: Synthetic oligonucleotide
  misc_feature 1..23: note = Human bone morphogenetic protein-2
  source 1..23: mol_type = other DNA
gccttaggaatcctagagttaac

SEQ ID NO: 59 - DNA, 25 bp, synthetic construct
  misc_feature 1..25: note = Description of Artificial Sequence: Synthetic oligonucleotide
  misc_feature 1..25: note = Human bone morphogenetic protein-2
  source 1..25: mol_type = other DNA
ggttgtattaaggaactatcaggac

SEQ ID NO: 60 - DNA, 25 bp, synthetic construct
  misc_feature 1..25: note = Description of Artificial Sequence: Synthetic oligonucleotide
  misc_feature 1..25: note = Human bone morphogenetic protein-2
  source 1..25: mol_type = other DNA
gtcctgatagttccttaatacaacc

SEQ ID NO: 61 - protein, 155 aa, synthetic construct
  REGION 1..155: note = Description of Artificial Sequence: Synthetic polypeptide
  REGION 1..155: note = Human fibroblast growth factor-1
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 62 - DNA, 21 bp, synthetic construct
  misc_feature 1..21: note = Description of Artificial Sequence: Synthetic oligonucleotide
  misc_feature 1..21: note = Human fibroblast growth factor-1
  source 1..21: mol_type = other DNA
aagaagcccagactcctctac

SEQ ID NO: 63 - DNA, 21 bp, synthetic construct
  misc_feature 1..21: note = Description of Artificial Sequence: Synthetic oligonucleotide
  misc_feature 1..21: note = Human fibroblast growth factor-1
  source 1..21: mol_type = other DNA
gtagaggagtctgggcttctt

SEQ ID NO: 64 - DNA, 21 bp, synthetic construct
  misc_feature 1..21: note = Description of Artificial Sequence: Synthetic oligonucleotide
  misc_feature 1..21: note = Human fibroblast growth factor-1
  source 1..21: mol_type = other DNA
catgcagagaggaattggttt

SEQ ID NO: 65 - DNA, 21 bp, synthetic construct
  misc_feature 1..21: note = Description of Artificial Sequence: Synthetic oligonucleotide
  misc_feature 1..21: note = Human fibroblast growth factor-1
  source 1..21: mol_type = other DNA
aaaccaattcctctctgcatg

SEQ ID NO: 66 - protein, 167 aa, synthetic construct
  REGION 1..167: note = Description of Artificial Sequence: Synthetic polypeptide
  REGION 1..167: note = Human Leptin
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 67 - DNA, 21 bp, synthetic construct
  misc_feature 1..21: note = Description of Artificial Sequence: Synthetic oligonucleotide
  misc_feature 1..21: note = Human Leptin
  source 1..21: mol_type = other DNA
cccatccaaaaggtccaagat

SEQ ID NO: 68 - DNA, 21 bp, synthetic construct
  misc_feature 1..21: note = Description of Artificial Sequence: Synthetic oligonucleotide
  misc_feature 1..21: note = Human Leptin
  source 1..21: mol_type = other DNA
atcttggaccttttggatggg

SEQ ID NO: 69 - DNA, 21 bp, synthetic construct
  misc_feature 1..21: note = Description of Artificial Sequence: Synthetic oligonucleotide
  misc_feature 1..21: note = Human Leptin
  source 1..21: mol_type = other DNA
gatgacaccaagaccctcatc

SEQ ID NO: 70 - DNA, 21 bp, synthetic construct
  misc_feature 1..21: note = Description of Artificial Sequence: Synthetic oligonucleotide
  misc_feature 1..21: note = Human Leptin
  source 1..21: mol_type = other DNA
gatgagggtcttggtgtcatc

SEQ ID NO: 71 - DNA, 21 bp, synthetic construct
  misc_feature 1..21: note = Description of Artificial Sequence: Synthetic oligonucleotide
  misc_feature 1..21: note = Human Leptin
  source 1..21: mol_type = other DNA
accctcatcaggacaattgtc

SEQ ID NO: 72 - DNA, 21 bp, synthetic construct
  misc_feature 1..21: note = Description of Artificial Sequence: Synthetic oligonucleotide
  misc_feature 1..21: note = Human Leptin
  source 1..21: mol_type = other DNA
gacaattgtcctgatgagggt

SEQ ID NO: 73 - DNA, 21 bp, synthetic construct
  misc_feature 1..21: note = Description of Artificial Sequence: Synthetic oligonucleotide
  misc_feature 1..21: note = Human Leptin
  source 1..21: mol_type = other DNA
accttatccaggatggaccag

SEQ ID NO: 74 - DNA, 21 bp, synthetic construct
  misc_feature 1..21: note = Description of Artificial Sequence: Synthetic oligonucleotide
  misc_feature 1..21: note = Human Leptin
  source 1..21: mol_type = other DNA
ctggtccatcctggataaggt

SEQ ID NO: 75 - protein, 209 aa, synthetic construct
  REGION 1..209: note = Description of Artificial Sequence: Synthetic polypeptide
  REGION 1..209: note = Human vascular endothelial growth factor A
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 76 - DNA, 28 bp, synthetic construct
  misc_feature 1..28: note = Description of Artificial Sequence: Synthetic oligonucleotide
  misc_feature 1..28: note = Human vascular endothelial growth factor A
  source 1..28: mol_type = other DNA
tacagcacaacagatgtgaatgcagacc

SEQ ID NO: 77 - DNA, 28 bp, synthetic construct
  misc_feature 1..28: note = Description of Artificial Sequence: Synthetic oligonucleotide
  misc_feature 1..28: note = Human vascular endothelial growth factor A
  source 1..28: mol_type = other DNA
ggtctgcattcacatctgttgtgctgta

SEQ ID NO: 78 - DNA, 27 bp, synthetic construct
  misc_feature 1..27: note = Description of Artificial Sequence: Synthetic oligonucleotide
  misc_feature 1..27: note = Human vascular endothelial growth factor A
  source 1..27: mol_type = other DNA
atccgcagacgtgtagatgttcctgca

SEQ ID NO: 79 - DNA, 27 bp, synthetic construct
  misc_feature 1..27: note = Description of Artificial Sequence: Synthetic oligonucleotide
  misc_feature 1..27: note = Human vascular endothelial growth factor A
  source 1..27: mol_type = other DNA
tgcaggaacatctacacgtctgcggat

SEQ ID NO: 80 - protein, 117 aa, synthetic construct
  REGION 1..117: note = Description of Artificial Sequence: Synthetic polypeptide
  REGION 1..117: note = Human prepro-GHRL
MPSPGTVCSLLLLGMLWLDLAMAGSSFLSPEHQRVQQRKESKKPPAKLQPRALAGWLRPEDGGQAEGAEDEMEVRFNAPFDVGIKLSGVQYQQHSQALGKFLQDILWEEAKEAPADK

SEQ ID NO: 81 - DNA, 21 bp, synthetic construct
  misc_feature 1..21: note = Description of Artificial Sequence: Synthetic oligonucleotide
  misc_feature 1..21: note = Human prepro-GHRL
  source 1..21: mol_type = other DNA
gccctggggaggtttcttcag

SEQ ID NO: 82 - DNA, 21 bp, synthetic construct
  misc_feature 1..21: note = Description of Artificial Sequence: Synthetic oligonucleotide
  misc_feature 1..21: note = Human prepro-GHRL
  source 1..21: mol_type = other DNA
ctgaagaaacctccccagggc

SEQ ID NO: 83 - protein, 28 aa, synthetic construct
  REGION 1..28: note = Description of Artificial Sequence: Synthetic peptide
  REGION 1..28: note = Human appetite stimulating hormone (Ghrelin)
GSSFLSPEHQRVQQRKESKKPPAKLQPR

SEQ ID NO: 84 - DNA, 31 bp, synthetic construct
  misc_feature 1..31: note = Description of Artificial Sequence: Synthetic oligonucleotide
  misc_feature 1..31: note = Human appetite stimulating hormone (Ghrelin)
  source 1..31: mol_type = other DNA
agtccagcagagaagggagtcgaagaagcca

SEQ ID NO: 85 - DNA, 31 bp, synthetic construct
  misc_feature 1..31: note = Description of Artificial Sequence: Synthetic oligonucleotide
  misc_feature 1..31: note = Human appetite stimulating hormone (Ghrelin)
  source 1..31: mol_type = other DNA
tggcttcttcgactcccttctctgctggact

SEQ ID NO: 86 - DNA, 31 bp, synthetic construct
  misc_feature 1..31: note = Description of Artificial Sequence: Synthetic oligonucleotide
  misc_feature 1..31: note = Human appetite stimulating hormone (Ghrelin)
  source 1..31: mol_type = other DNA
agaaaggagtcgaggaagccaccagccaagc

SEQ ID NO: 87 - DNA, 31 bp, synthetic construct
  misc_feature 1..31: note = Description of Artificial Sequence: Synthetic oligonucleotide
  misc_feature 1..31: note = Human appetite stimulating hormone (Ghrelin)
  source 1..31: mol_type = other DNA
gcttggctggtggcttcctcgactcctttct

SEQ ID NO: 88 - DNA, 31 bp, synthetic construct
  misc_feature 1..31: note = Description of Artificial Sequence: Synthetic oligonucleotide
  misc_feature 1..31: note = Human appetite stimulating hormone (Ghrelin)
  source 1..31: mol_type = other DNA
agaaaggagtcgaagaggccaccagccaagc

SEQ ID NO: 89 - DNA, 31 bp, synthetic construct
  misc_feature 1..31: note = Description of Artificial Sequence: Synthetic oligonucleotide
  misc_feature 1..31: note = Human appetite stimulating hormone (Ghrelin)
  source 1..31: mol_type = other DNA
gcttggctggtggcctcttcgactcctttct

SEQ ID NO: 90 - DNA, 30 bp, synthetic construct
  misc_feature 1..30: note = Description of Artificial Sequence: Synthetic oligonucleotide
  misc_feature 1..30: note = Human appetite stimulating hormone (Ghrelin)
  source 1..30: mol_type = other DNA
aagaagccaccagccaggctgcagccccga

SEQ ID NO: 91 - DNA, 30 bp, synthetic construct
  misc_feature 1..30: note = Description of Artificial Sequence: Synthetic oligonucleotide
  misc_feature 1..30: note = Human appetite stimulating hormone (Ghrelin)
  source 1..30: mol_type = other DNA
tcggggctgcagcctggctggtggcttctt

SEQ ID NO: 92 - protein, 31 aa, synthetic construct
  REGION 1..31: note = Description of Artificial Sequence: Synthetic polypeptide
  REGION 1..31: note = Human glucagon-like peptide-1 (GLP-1)
HAEGTFTSDVSSYLEGQAAKEFIAWLVKGRG

SEQ ID NO: 93 - DNA, 18 bp, synthetic construct
  misc_feature 1..18: note = Description of Artificial Sequence: Synthetic oligonucleotide
  misc_feature 1..18: note = Human glucagon-like peptide-1 (GLP-1)
  source 1..18: mol_type = other DNA
aagctgccagggaattca

SEQ ID NO: 94 - DNA, 18 bp, synthetic construct
  misc_feature 1..18: note = Description of Artificial Sequence: Synthetic oligonucleotide
  misc_feature 1..18: note = Human glucagon-like peptide-1 (GLP-1)
  source 1..18: mol_type = other DNA
tgaattccctggcagctt

SEQ ID NO: 95 - DNA, 17 bp, synthetic construct
  misc_feature 1..17: note = Description of Artificial Sequence: Synthetic oligonucleotide
  misc_feature 1..17: note = Human glucagon-like peptide-1 (GLP-1)
  source 1..17: mol_type = other DNA
ttggctggtgagaggcc

SEQ ID NO: 96 - DNA, 17 bp, synthetic construct
  misc_feature 1..17: note = Description of Artificial Sequence: Synthetic oligonucleotide
  misc_feature 1..17: note = Human glucagon-like peptide-1 (GLP-1)
  source 1..17: mol_type = other DNA
ggcctctcaccagccaa

SEQ ID NO: 97 - protein, 470 aa, synthetic construct
  REGION 1..470: note = Description of Artificial Sequence: Synthetic polypeptide
  REGION 1..470: note = Human IgG heavy chain
MDWTWRFLFVVAAATGVQSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGLE

SEQ ID NO: 98 - DNA, 30 bp, synthetic construct
  misc_feature 1..30: note = Description of Artificial Sequence: Synthetic oligonucleotide
  misc_feature 1..30: note = Human IgG heavy chain
  source 1..30: mol_type = other DNA
acaaaggtggacaggaaggtggagcccaag

SEQ ID NO: 99 - DNA, 30 bp, synthetic construct
  misc_feature 1..30: note = Description of Artificial Sequence: Synthetic oligonucleotide
  misc_feature 1..30: note = Human IgG heavy chain
  source 1..30: mol_type = other DNA
cttgggctccaccttcctgtccacctttgt

SEQ ID NO: 100 - DNA, 34 bp, synthetic construct
  misc_feature 1..34: note = Description of Artificial Sequence: Synthetic oligonucleotide
  misc_feature 1..34: note = Human IgG heavy chain
  source 1..34: mol_type = other DNA
gagtataagtgcagggtgtccaataaggccctgc

SEQ ID NO: 101 - DNA, 34 bp, synthetic construct
  misc_feature 1..34: note = Description of Artificial Sequence: Synthetic oligonucleotide
  misc_feature 1..34: note = Human IgG heavy chain
  source 1..34: mol_type = other DNA
gcagggccttattggacaccctgcacttatactc

SEQ ID NO: 102 - DNA, 34 bp, synthetic construct
  misc_feature 1..34: note = Description of Artificial Sequence: Synthetic oligonucleotide
  misc_feature 1..34: note = Human IgG heavy chain
  source 1..34: mol_type = other DNA
ctttctgtatagcaggctgaccgtggataagtcc

SEQ ID NO: 103 - DNA, 34 bp, synthetic construct
  misc_feature 1..34: note = Description of Artificial Sequence: Synthetic oligonucleotide
  misc_feature 1..34: note = Human IgG heavy chain
  source 1..34: mol_type = other DNA
ggacttatccacggtcagcctgctatacagaaag

SEQ ID NO: 104 - protein, 238 aa, synthetic construct
  REGION 1..238: note = Description of Artificial Sequence: Synthetic polypeptide
  REGION 1..238: note = Human IgG light chain
MDMRVPAQLLGLLLLWLSGARCDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLE

SEQ ID NO: 105 - DNA, 30 bp, synthetic construct
  misc_feature 1..30: note = Description of Artificial Sequence: Synthetic oligonucleotide
  misc_feature 1..30: note = Human IgG light chain
  source 1..30: mol_type = other DNA
cctggcaaggccccaaggctgctgatctac

SEQ ID NO: 106 - DNA, 30 bp, synthetic construct
  misc_feature 1..30: note = Description of Artificial Sequence: Synthetic oligonucleotide
  misc_feature 1..30: note = Human IgG light chain
  source 1..30: mol_type = other DNA
gtagatcagcagccttggggccttgccagg

SEQ ID NO: 107 - DNA, 30 bp, synthetic construct
  misc_feature 1..30: note = Description of Artificial Sequence: Synthetic oligonucleotide
  misc_feature 1..30: note = Human IgG light chain
  source 1..30: mol_type = other DNA
acaaaggtggagatcaggaggaccgtggcc

SEQ ID NO: 108 - DNA, 30 bp, synthetic construct
  misc_feature 1..30: note = Description of Artificial Sequence: Synthetic oligonucleotide
  misc_feature 1..30: note = Human IgG light chain
  source 1..30: mol_type = other DNA
ggccacggtcctcctgatctccacctttgt

SEQ ID NO: 109 - DNA, 30 bp, synthetic construct
  misc_feature 1..30: note = Description of Artificial Sequence: Synthetic oligonucleotide
  misc_feature 1..30: note = Human IgG light chain
  source 1..30: mol_type = other DNA
gccaaggtgcagtggagggtggataacgcc

SEQ ID NO: 110 - DNA, 30 bp, synthetic construct
  misc_feature 1..30: note = Description of Artificial Sequence: Synthetic oligonucleotide
  misc_feature 1..30: note = Human IgG light chain
  source 1..30: mol_type = other DNA
ggcgttatccaccctccactgcaccttggc

SEQ ID NO: 111 - DNA, 597 bp, Homo sapiens
  source 1..597: mol_type = genomic DNA
atgccagtgcctgctctgtgcctgctctgggccctggcaatggtgacccggcctgcctcagcggcccccatgggcggcccagaactggcacagcatgaggagctgaccctgctcttccatgggaccctgcagctgggccaggccctcaacggtgtgtacaggaccacggagggacggctgacaaaggccaggaacagcctgggtctctatggccgcacaatagaactcctggggcaggaggtcagccggggccgggatgcagcccaggaacttcgggcaagcctgttggagactcagatggaggaggatattctgcagctgcaggcagaggccacagctgaggtgctgggggaggtggcccaggcacagaaggtgctacgggacagcgtgcagcggctagaagtccagctgaggagcgcctggctgggccctgcctaccgagaatttgaggtcttaaaggctcacgctgacaagcagagccacatcctatgggccctcacaggccacgtgcagcggcagaggcgggagatggtggcacagcagcatcggctgcgacagatccaggagagactccacacagcggcgctcccagcctga

SEQ ID NO: 112 - DNA, 654 bp, Homo sapiens
  source 1..654: mol_type = genomic DNA
atggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 113 - DNA, 333 bp, Homo sapiens
  source 1..333: mol_type = genomic DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 114 - DNA, 567 bp, Homo sapiens
  source 1..567: mol_type = genomic DNA
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 115 - DNA, 624 bp, Homo sapiens
  source 1..624: mol_type = genomic DNA
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctga

SEQ ID NO: 116 - DNA, 564 bp, Homo sapiens
  source 1..564: mol_type = genomic DNA
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga

SEQ ID NO: 117 - DNA, 582 bp, Homo sapiens
  source 1..582: mol_type = genomic DNA
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 118 - DNA, 1191 bp, Homo sapiens
  source 1..1191: mol_type = genomic DNA
atggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctag

SEQ ID NO: 119 - DNA, 468 bp, Homo sapiens
  source 1..468: mol_type = genomic DNA
atggctgaaggagaaatcaccaccttcacagccctgacagaaaagtttaacctgcctccagggaactacaagaagcccaaactcctctactgtagcaatggaggccacttcctgaggatccttcctgatggcactgtggatgggaccagggacaggtctgaccagcacattcagctgcagctcagtgctgaatcagtgggagaggtgtacattaagagtacagagactggccagtacttggccatggacactgatggacttctgtatggctcacagacaccaaatgaggaatgcttgttcctggaaaggctggaggagaaccattacaacacctatatctccaagaagcatgcagagaagaattggtttgttggcctcaagaagaatgggagctgcaaaagaggtcctagaactcactatggccagaaagccatcttgtttctccccctgccagtctcttctgactaa

SEQ ID NO: 120 - DNA, 504 bp, Homo sapiens
  source 1..504: mol_type = genomic DNA
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctga

SEQ ID NO: 121 - DNA, 630 bp, Homo sapiens
  source 1..630: mol_type = genomic DNA
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccgtccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 122 - DNA, 354 bp, Homo sapiens
  source 1..354: mol_type = genomic DNA
atgccctccccagggaccgtctgcagcctcctgctcctcggcatgctctggctggacttggccatggcaggctccagcttcctgagccctgaacaccagagagtccagcagagaaaggagtcgaagaagccaccagccaagctgcagccccgagctctagcaggctggctccgcccggaagatggaggtcaagcagaaggggcagaggatgaaatggaagtccggttcaacgccccctttgatgttggaatcaagctgtcaggggttcagtaccagcagcacagccaggccctggggaagtttcttcaggacatcctctgggaagaggccaaagaggccccagccgacaagtga

SEQ ID NO: 123 - DNA, 84 bp, Homo sapiens
  source 1..84: mol_type = genomic DNA
ggctccagcttcctgagccctgaacaccagagagtccagcagagaaaggagtcgaagaagccaccagccaagctgcagccccga

SEQ ID NO: 124 - DNA, 93 bp, Homo sapiens
  source 1..93: mol_type = genomic DNA
catgctgaagggacctttaccagtgatgtaagttcttatttggaaggccaagctgccaaggaattcattgcttggctggtgaaaggccgagga

SEQ ID NO: 125 - DNA, 1413 bp, synthetic construct
  misc_feature 1..1413: note = Description of Artificial Sequence: Synthetic polynucleotide
  source 1..1413: mol_type = other DNA
atggactggacctggaggttcctgtttgtggtggcagcagcaacaggagtgcagtccgaggtgcagctggtggagtctggaggaggcctggtgcagccaggaggctccctgcgcctgtcttgcgccgccagcggcttcaacatcaaggatacctacatccactgggtgcggcaggcacctggcaagggcctggagtgggtggccagaatctatccaaccaatggctacacacggtatgccgactccgtgaagggccggttcaccatctccgccgatacctctaagaacacagcctacctgcagatgaatagcctgcgggccgaggacacagccgtgtactattgctccagatggggcggcgacggcttctacgccatggattattggggccagggcaccctggtgacagtgagctccgccagcaccaagggcccttccgtgtttccactggccccctctagcaagtctaccagcggaggaacagccgccctgggatgtctggtgaaggattatttcccagagcccgtgacagtgagctggaactccggcgccctgacctccggagtgcacacatttcctgccgtgctgcagtcctctggcctgtacagcctgagctccgtggtgaccgtgccatctagctccctgggcacccagacatatatctgcaacgtgaatcacaagccatctaatacaaaggtggacaagaaggtggagcccaagagctgtgataagacccacacatgccccccttgtcctgcaccagagctgctgggaggaccaagcgtgttcctgtttccacccaagcctaaggacaccctgatgatctcccggaccccagaggtgacatgcgtggtggtggacgtgtctcacgaggatcccgaggtgaagttcaactggtacgtggatggcgtggaggtgcacaatgccaagaccaagcccagggaggagcagtacaactctacctatcgcgtggtgagcgtgctgacagtgctgcaccaggactggctgaacggcaaggagtataagtgcaaggtgtccaataaggccctgcccgcccctatcgagaagaccatcagcaaggccaagggccagcctagggagccacaggtgtacacactgcctccatcccgcgaggagatgaccaagaaccaggtgtctctgacatgtctggtgaagggcttctatccctctgacatcgccgtggagtgggagagcaatggccagcctgagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcccggcctggagtga

SEQ ID NO: 126 - DNA, 717 bp, synthetic construct
  misc_feature 1..717: note = Description of Artificial Sequence: Synthetic polynucleotide
  source 1..717: mol_type = other DNA
atggacatgagggtgccagcacagctgctgggcctgctgctgctgtggctgagcggagccagatgcgatatccagatgacccagtcccccagctccctgtctgccagcgtgggcgacagggtgaccatcacatgtcgcgcctcccaggatgtgaacaccgccgtggcctggtaccagcagaagcctggcaaggccccaaagctgctgatctactccgcctctttcctgtatagcggagtgccctcccggtttagcggctccagatctggcaccgacttcaccctgacaatctctagcctgcagcctgaggattttgccacatactattgccagcagcactataccacaccccctaccttcggccagggcacaaaggtggagatcaagaggaccgtggccgccccttccgtgttcatctttccaccctctgacgagcagctgaagtctggcacagccagcgtggtgtgcctgctgaacaacttctacccacgcgaggccaaggtgcagtggaaggtggataacgccctgcagagcggcaattcccaggagtctgtgaccgagcaggacagcaaggattccacatattctctgtcctctaccctgacactgtctaaggccgactacgagaagcacaaggtgtatgcatgcgaggtgacccaccagggcctgagctccccagtgacaaagagcttcaatcggggcgagtgtctggagtga